(19) United States
(12) Patent Application Publication
(10) Pub. No.: US 2025/0235554 A1
(43) Pub. Date: Jul. 24, 2025

(54) METHODS FOR IMPROVING ADENO-ASSOCIATED VIRUS (AAV) DELIVERY

(71) Applicant: Novartis AG, Basel (CH)

(72) Inventor: Keith MANSFIELD, Shrewsbury, MA (US)

(21) Appl. No.: 18/703,559

(22) PCT Filed: Oct. 24, 2022
(86) PCT No.: PCT/IB2022/060189

§ 371 Date: Apr. 22, 2024

Related U.S. Application Data
(60) Provisional application No. 63/271,487, filed on Oct. 25, 2021.

(51) Int. Cl. A61K 48/00; A61K 9/00; A61K 31/08; A61K 31/135; A61K 31/4164; A61K 31/433; A61K 31/44; A61K 38/17; A61K 45/06; A61K 47/02; A61K 47/26; C12N 15/113; C12N 15/86

(52) U.S. Cl. CPC A61K 48/005; A61K 9/0048; A61K 31/08; A61K 31/135; A61K 31/4164; A61K 31/433; A61K 31/44; A61K 38/1709; A61K 45/06; A61K 47/02; A61K 47/26; A61K 48/0075; A61K 48/0083; C12N 15/1137; C12N 15/86; C12N 2310/14; C12N 2310/531; C12N 2750/14122; C12N 2750/14143; C12N 2830/50; C12Y 115/01001

(57) ABSTRACT

Provided herein are methods for improving delivery of a pharmaceutical composition to the central nervous system of a subject in need thereof, the method comprising administering to the subject an agent that enhances glymphatic influx in combination with the pharmaceutical composition.

### FIELD

The present disclosure is directed to improving delivery of pharmaceutical compositions to target tissues, such as the central nervous system, by modulating glymphatic influx.

### BACKGROUND

Various therapeutic agents have been developed for treating central nervous system (CNS) diseases. However, delivery therapeutic agents to the brain is severely limited by the largely impermeable blood-brain barrier (BBB) and poor penetration of the therapeutic agents to the brain. AAV vectors have emerged as a promising approach to treat diverse genetically determined diseases but require delivery to and transgene expression in specific tissues and cell types to achieve efficacy and avoid unwanted toxicities. This process first requires exposure of the intended tissue to the vector and subsequently vector tropism for the intended targeted cell type. For diseases requiring transduction of the central nervous system several routes of delivery have been attempted to achieve adequate tissue exposure including systemic intravascular administration as well as direct injection into the intrathecal and intraventricular spaces. Following intravascular administration, the vector must cross the blood brain barrier, which appears to limit exposure to the brain parenchyma for many AAV serotypes. Direct injection into the intrathecal and intraventricular space bypasses the blood brain barrier but the mechanism by which the vector distributes from the cerebrospinal fluid to the brain parenchyma remains undefined.

To further elucidate the mechanism of distribution following intrathecal administration of AAV vectors, a non-human primate study in cynomolgus macaques was conducted. The results suggest a low level of AAV transduction in the brain. The glymphatic system, a network of perivascular spaces promoting fluid exchange between CSF and interstitial space, can be utilized to enhance drug delivery from the CSF to the parenchyma. Glymphatic flow is highest during sleep and anesthesia regimens that induce a slow-wave sleep-like state. (Lilius T O, et al. Dexmedetomidine enhances glymphatic brain delivery of intrathecally administered drugs. J Control Release. 2019 Jun. 28; 304:29-38). Thus, there is a need for improving AAV delivery to the brain by modulating glymphatic flow.

### SUMMARY

The present disclosure provides methods of improving delivery of pharmaceutical compositions to target tissues, such as the central nervous system, by modulating glymphatic influx. The glymphatics are a recently recognized system by which CSF is drawn into the deeper regions of the brain along periarterial spaces formed by vessel adjacent astrocytes where CSF may exchange with the interstitial fluid prior to exiting the brain in an equivalent perivenule space. This system is thought to play a major role in the movement of fluid and removal of macromolecules from the brain parenchyma. Larger particles such as lipoproteins which are of equivalent size to AAV vectors move through the glymphatic system. It has been discovered that the AAV distribution patterns in the brain are consistent with limited diffusion of vector across membranes lining the brain surface and vector entry occurring primarily through glymphatic influx. It is unexpectly discovered that AAV delivery to the brain can be improved by modulating glymphatic influx. Enhancing glymphatic influx can also reduce variable brain distribution of viral vectors among a population of patients treated with a pharmaceutical composition comprising the viral vectors, as well as reduce liver and/or DRG toxicity associated with AAV gene therapy.

In one aspect, the present disclosure provides methods for improving delivery of a pharmaceutical composition to the central nervous system of a subject in need thereof, the method comprising administering to the subject an agent that enhances glymphatic influx in combination with the pharmaceutical composition.

In some embodiments, the agent is administered concurrently or sequentially with the pharmaceutical composition. In some embodiments, the agent is administered prior to the administration of the pharmaceutical composition. In some embodiments, the agent is administered after the administration of the pharmaceutical composition. In some embodiments, the pharmaceutical composition is administered by intrathecal (IT), intra-cisterna magna (ICM), and/or intracerebroventricular (ICV) administration. In some embodiments, the agent is administered by intravenous infusion, intravenous injection, inhalation, intraperitoneal, oral, subcutaneous or intramuscular routes.

In some embodiments, the agent promotes interstitial fluid circulation within the blood-brain barrier, e.g., wherein the agent comprises an Aquaporin 4 (AQP4) facilitator, e.g. TGN-073. In some embodiments, the agent comprises a compound that upregulates AQP4 expression (e.g. sevoflurane) or alters subcellular localization of AQP4.

In some embodiments, the agent comprises an α-2 adrenergic agonist, e.g. clonidine, cizanidine, or dexmedetomidine (e.g. Precedex or Dexdomitor). In some embodiments, the agent comprises one or more FDA approved anesthetics that enhance glymphatic influx. In some embodiments, the anesthetic is ketamine, dexmedetomidine, orxylazine, or a combination thereof. In preferred embodiments, the agent comprises a combination of ketamine and dexmedetomidine.

In some embodiments, the subject is administered first with ketamine, followed by administration of the pharmaceutical composition, and followed by administration of dexmedetomidine.

In some embodiments, the subject is administered first with ketamine, followed by administration of the pharmaceutical composition, followed by administration of dexmedetomidine, and followed by administration of sevoflurane.

In some embodiments, ketamine is administered about 1, 2, 3, 4, 5, 10, 15, 20, 25, 30, 35, 40, 45, 50, 55, or 60 minutes prior to, and preferably about 10 to 15 minutes prior to the administration of the pharmaceutical composition.

In some embodiments, ketamine is administered at about 100 mg/kg, about 90 mg/kg, about 80 mg/kg, about 70 mg/kg, about 60 mg/kg, about 50 mg/k, about 40 mg/kg, about 30 mg/kg, about 20 mg/kg, about 10 mg/kg, about 9 mg/kg, about 8 mg/kg, about 7 mg/kg, about 6 mg/kg, about 5 mg/kg, about 4 mg/kg, about 3 mg/kg, about 2 mg/kg, about 1 mg/kg, preferable at about 10 mg/kg.

In some embodiments, dexmedetomidine is administered at about 1 mg/kg, about 0.9 mg/kg, about 0.8 mg/kg, about 0.7 mg/kg, about 0.6 mg/kg, about 0.5 mg/k, about 0.4 mg/kg, about 0.3 mg/kg, about 0.2 mg/kg, about 0.1 mg/kg, about 0.09 mg/kg, about 0.08 mg/kg, about 0.07 mg/kg, about 0.06 mg/kg, about 0.05 mg/kg, about 0.04 mg/kg, about 0.03 mg/kg, about 0.02 mg/kg, about 0.01 mg/kg, about 0.009 mg/kg, about 0.008 mg/kg, about 0.007 mg/kg, about 0.006 mg/kg about 0.005 mg/kg, and preferable at about 0.02 mg/kg.

In some embodiments, the subject is additionally administered sevolurane following the administration of dexmedetomidine.

In some embodiments, sevolurane is administered as an inhalant.

In some preferred embodiments, the subject is administered ketamine (10 mg/kg) 10 to 15 prior to dosing followed by dexmedetomidine (0.02 mg/kg). The subject is then maintained in dorsal recumbence with hind limbs elevated (Trendelenburg like position) for 10 to 15 minutes after completion of administration of ketamine and dexmedetomidine. The subject is additionally administered administered atipamezole (0.2 mg/kg IM), with dosing occurs at a standard time 8-10 AM.

In some preferred embodiments, the subject is administered with ketamine (10 mg/kg) 10 to 15 minutes prior to dosing followed by dexmedetomidine (0.02 mg/kg) followed by sevoflurane inhalant anesthesia.

In some embodiments, the agent induces plasma hypertonicity. In some embodiments, the agent comprises hypertonic saline (e.g., Sodium chloride with or without sodium acetate) or mannitol. In preferred embodiments, the agent comprises hypertonic saline with or without sodium acetate. In some embodiments, the hypertonic saline is 2% NaCl, 3% NaCl, 5% NaCl, 7% NaCl or 23% NaCl, and preferably 3% NaCl. In some embodiments, the 3% NaCl is administered at about 2-3.5 ml/kg.

In some embodiments, the agent is administered by intravenous or infusion injection about 1, 2, 3, 4, 5, 6, 7, 8, 9, 10, 15, 20, preferably about 5 minutes prior or after to administration of the pharmaceutical composition, and optionally wherein the administration can be repeated.

In some embodiments, the agent enhances glymphatic influx by increasing slow wave sleep. In some embodiments, the agent is selected from the group consisting of: Tiagabine, Gaboxadol, Gabapentin, Pregabalin, GHB, Ritanserin, Eplivanserin, Mirtazapine, Olanzapine, and Trazodone, or a combination thereof.

In some embodiments, wherein the agent comprises VEGF, such as VEGF-C. In some embodiments, the VEGF-C comprises: (i) an amino acid sequence of any one of the sequences provided in Table 1 or a sequence with at least 95% sequence identity thereto, optionally wherein the sequence comprises or does not comprise a linker (e.g., a glycine-serine linker) and/or a his tag; and/or (ii) the amino acid substitution of C137A, numbered according to SEQ ID NO: 1.

In some embodiments, the subject is maintained in a position with hind limbs elevated, e.g. Trendelenburg like position, for about 1-2 hours after the administration of the pharmaceutical composition.

In some embodiments, the pharmaceutical composition comprises viral vectors, antibody, antisense oligonucleotide, or nanoparticles.

In some embodiments, the pharmaceutical composition comprises adeno-associated virus (AAV) viral vectors.

In some embodiments, the AAV viral vector comprise a capsid protein derived from AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-10, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV-17/rh.48, AAV-18/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r 11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAV A3.3, AAV A3.4, AAV A3.5, AAV A3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.6, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC 12, AAV-2-pre-miRNA-1O1, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPEN AAV 10 and/or Japanese AAV 10 serotypes, and variants thereof.

In some embodiments, the AAV viral vector comprise a capsid protein derived from AAV9.

In some embodiments, the AAV viral vector comprises a polynucleotide encoding a survival motor neuron (SMN) protein.

In some embodiments, the AAV viral vector comprises a polynucleotide encoding a methyl-CpG-binding protein 2 (MECP2) protein.

In some embodiments, the AAV viral vector comprises a polynucleotide encoding a short hairpin RNA (shRNA) targeting superoxide dismutase 1 (SOD1).

In some embodiments, the AAV viral vector comprises two ITRs (e.g. a modified AAV2 ITR and an unmodified AAV2 ITR), a promoter (e.g. a chicken beta-actin (CB) promoter), an enhancer (e.g. a cytomegalovirus (CMV) immediate/early enhancer), an intro (e.g. a modified SV40 late 16s intron), a polyadenylation signal (e.g. a bovine growth hormone (BGH) polyadenylation signal).

In some embodiments, the pharmaceutical composition comprises between 1×10¹⁰ and 1×10¹⁵ viral vector genomes, such as 1×10¹², 5×10¹², 1×10¹³, 5×10¹³, 1×10¹⁴, 5×10¹⁴, 1×10¹⁵ viral vector genomes.

In some embodiments, the pharmaceutical composition comprises between 1×10¹⁰ to 1×10¹⁵ vector genome per milliliter (vg/ml), such as 1×10¹², 5×10¹², 1×10¹³, 5×10¹³, 1×10¹⁴, 5×10¹⁴, 1×10¹⁵ vector genome per milliliter (vg/ml).

In another aspect, the present disclosure provides methods of treating a neurological disease, comprising administering a pharmaceutical composition according any one of the proceeding embodiments to a subject in need thereof, wherein the pharmaceutical composition comprises an AAV encoding a gene associated with the neurological disease, wherein the administration of the pharmaceutical composition coincide with CSF influx during sleep cycle.

In some embodiments, the pharmaceutical composition is administered when the subject goes to sleep, e.g. as indicated by electroencephalogram (EEG) monitoring.

In some embodiments, the subject is administered a sleep enhancing drug in combination with the pharmaceutical combination.

In some embodiments, the sleep enhancing drug is selected from the group consisting of: Tiagabine, Gaboxadol, Gabapentin, Pregabalin, GHB, Ritanserin, Eplivanserin, Mirtazapine, Olanzapine, and Trazodone, or a combination thereof.

In another aspect, the present disclosure provides methods for improving the transduction efficiency and/or distribution of a neurodegenerative therapeutic agent in brain comprising administering the neurodegenerative therapeutic agent, in a subject in need thereof, in combination with a second agent that enhances glymphatic influx, to thereby improve transduction efficiency of the neurodegenerative therapeutic agent in the subject.

In some embodiments, the neurodegenerative therapeutic agent is a viral vector, an antibody, an antisense oligonucleotide, or a nanoparticle that targets the CNS.

In some embodiments, the second agent is administered concurrently or sequentially with the neurodegenerative therapeutic agent. In some embodiments, the second agent is administered prior to the neurodegenerative therapeutic agent. In some embodiments, the second agent is administered after the neurodegenerative therapeutic agent.

In some embodiments, the neurodegenerative therapeutic agent is administered by intrathecal (IT) by intra-cisterna magna (ICM) and/or ICV administration by bolus, slow bolus and/or infusion through implanted intrathecal or intraventricular catheter.

In some embodiments, the second agent is administered by intravenous infusion, intravenous injection and/or inhalation.

In some embodiments, the second agent comprises an AQP4 facilitator, e.g. TGN-073. In some embodiments, the second agent comprises a compound that upregulates AQP4, e.g. sevoflurane.

In some embodiments, the second agent comprises an α-2 adrenergic agonist, e.g. clonidine, cizanidine, or dexmedetomidine (e.g. Precedex or Dexdomitor).

In some embodiments, the second agent comprises one or more FDA approved anesthetics that enhance glymphatic influx. In some embodiments, the anesthetic is ketamine, dexmedetomidine, orxylazine, or a combination thereof.

In preferred embodiments, wherein the second agent comprises a combination of ketamine and dexmedetomidine.

In some embodiments, ketamine is administered about 1, 2, 3, 4, 5, 10, 15, 20, 25, 30, 35, 40, 45, 50, 55, or 60 minutes prior to, and preferably about 10 to 15 minutes prior to the administration of the neurodegenerative therapeutic agent.

In some embodiments, ketamine is administered at about about 100 mg/kg, about 90 mg/kg, about 80 mg/kg, about 70 mg/kg, about 60 mg/kg, about 50 mg/k, about 40 mg/kg, about 30 mg/kg, about 20 mg/kg, about 10 mg/kg, about 9 mg/kg, about 8 mg/kg, about 7 mg/kg, about 6 mg/kg, about 5 mg/kg, about 4 mg/kg, about 3 mg/kg, about 2 mg/kg, about 1 mg/kg, preferable at about 10 mg/kg.

In some embodiments, dexmedetomidine is administered at about 1 mg/kg, about 0.9 mg/kg, about 0.8 mg/kg, about 0.7 mg/kg, about 0.6 mg/kg, about 0.5 mg/k, about 0.4 mg/kg, about 0.3 mg/kg, about 0.2 mg/kg, about 0.1 mg/kg, about 0.09 mg/kg, about 0.08 mg/kg, about 0.07 mg/kg, about 0.06 mg/kg, about 0.05 mg/kg, about 0.04 mg/kg, about 0.03 mg/kg, about 0.02 mg/kg, about 0.01 mg/kg, about 0.009 mg/kg, about 0.008 mg/kg, about 0.007 mg/kg, about 0.006 mg/kg about 0.005 mg/kg, and preferable at about 0.02 mg/kg.

In some embodiments, the subject is additionally administered sevolurane following the administration of dexmedetomidine. In some embodiments, sevolurane is administered as an inhalant.

In some preferred embodiments, the subject is administered ketamine (10 mg/kg) 10 to 15 prior to dosing followed by dexmedetomidine (0.02 mg/kg). The subject is then maintained in dorsal recumbence with hind limbs elevated (Trendelenburg like position) for 10 to 15 minutes after completion of administration of ketamine and dexmedetomidine. The subject is additionally administered administered atipamezole (0.2 mg/kg IM), with dosing occurs at a standard time 8-10 AM.

In some preferred embodiments, the subject is administered with ketamine (10 mg/kg) 10 to 15 minutes prior to dosing followed by dexmedetomidine (0.02 mg/kg) followed by sevoflurane inhalant anesthesia.

In some embodiments, the second agent induces plasma hypertonicity. In some embodiments, the second agent comprises hypertonic saline (e.g., Sodium chloride with or without sodium acetate) or mannitol. In some embodiments, the second agent comprises hypertonic saline with or without sodium acetate. In some embodiments, the hypertonic saline is 2% NaCl, 3% NaCl, 5% NaCl, 7% NaCl or 23% NaCl, and preferably 3% NaCl. In some embodiments, the 3% NaCl is administered at about 2-3.5 ml/kg.

In some embodiments, the agent is administered by intravenous or infusion injection about 1, 2, 3, 4, 5, 6, 7, 8, 9, 10, 15, 20, preferably about 5 minutes prior or after to administration of the pharmaceutical composition, and optionally wherein the administration can be repeated.

In some embodiments, the second agent enhances glymphatic influx by increasing slow wave sleep. In some embodiments, the second agent is selected from the group consisting of: Tiagabine, Gaboxadol, Gabapentin, Pregabalin, GHB, Ritanserin, Eplivanserin, Mirtazapine, Olanzapine, and Trazodone, or a combination thereof.

In some embodiments, the second agent comprises, VEGF, such as VEGF-C. In some embodiments, the VEGF-C comprises: (i) an amino acid sequence of any one of the sequences provided in Table 1 or a sequence with at least 95% sequence identity thereto, optionally wherein the sequence comprises or does not comprise a linker (e.g., a glycine-serine linker) and/or a his tag; and/or (ii) the amino acid substitution of C137A, numbered according to SEQ ID NO: 1.

In some embodiments, the subject is maintained in a position with hind limbs elevated, e.g. Trendelenburg like position, for about 1 to 2 hours after the administration of the pharmaceutical composition.

In some embodiments, the neurodegenerative therapeutic agent is an adeno-associated virus (AAV) viral vector.

In some embodiments, the AAV viral vector comprise a capsid protein derived from AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV 12, AAV16.3, AAV24.1, AAV27.3, AAV42.12, AAV42-lb, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV-17/rh.48, AAV-18/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r 11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAV A3.3, AAV A3.4, AAV A3.5, AAV A3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC 12, AAV-2-pre-miRNA-1O1, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPEN AAV 10 and/or Japanese AAV 10 serotypes, and variants thereof.

In some embodiments, the AAV viral vector comprise a capsid protein derived from AAV9.

In some embodiments, the AAV viral vector comprises a polynucleotide encoding a survival motor neuron (SMN) protein.

In some embodiments, the AAV viral vector comprises a polynucleotide encoding a methyl-CpG-binding protein 2 (MECP2) protein.

In some embodiments, the AAV viral vector comprises a polynucleotide encoding a short hairpin RNA (shRNA) targeting superoxide dismutase 1 (SOD1).

In some embodiments, the AAV viral vector comprises two ITRs (e.g. a modified AAV2 ITR and an unmodified AAV2 ITR), a promoter (e.g. a chicken beta-actin (CB) promoter), an enhancer (e.g. a cytomegalovirus (CMV) immediate/early enhancer), an intro (e.g. a modified SV40 late 16s intron), a polyadenylation signal (e.g. a bovine growth hormone (BGH) polyadenylation signal).

In some embodiments, the pharmaceutical composition comprises between 1×10¹⁰ and 1×10¹⁵ viral vector genomes, such as 1×10¹², 5×10¹², 1×10¹³, 5×10¹³, 1×10¹⁴, 5×10¹⁴, 1×10¹⁵ viral vector genomes.

In some embodiments, the composition comprises between 1×10¹⁰ to 1×10¹⁵ vector genome per milliliter (vg/ml), such as 1×10¹², 5×10¹², 1×10¹³, 5×10¹³, 1×10¹⁴, 5×10¹⁴, 1×10¹⁵ vector genome per milliliter (vg/ml).

In another aspect, the present disclosure provides methods of increasing efficacy of an intrathecally delivered pharmaceutical composition, the method comprising administering to a subject in need thereof the pharmaceutical composition, in combination with an agent that enhances glymphatic influx.

In some embodiments, the agent is administered concurrently or sequentially with the composition. In some embodiments, the agent is administered prior to the administration of the composition. In some embodiments, the agent is administered after the administration of the composition. In some embodiments, the agent is administered by intravenous infusion, intravenous injection and/or inhalation.

In some embodiments, the agent comprises an AQP4 facilitator, e.g. TGN-073.

In some embodiments, the agent comprises a compound that upregulates AQP4, e.g. sevoflurane.

In some embodiments, the agent comprises an α-2 adrenergic agonist, e.g. clonidine, cizanidine, or dexmedetomidine (e.g. Precedex or Dexdomitor).

In some embodiments, the agent comprises one or more FDA approved anesthetics that enhance glymphatic influx. In some embodiments, wherein the anesthetic is ketamine, dexmedetomidine, orxylazine, or a combination thereof.

In preferred embodiments, the agent comprises a combination of ketamine and dexmedetomidine. In some embodiments, the subject is administered first with ketamine, followed by administration of the pharmaceutical composition, and followed by administration of dexmedetomidine. In some embodiments, the subject is administered first with ketamine, followed by administration of the pharmaceutical composition, followed by administration of dexmedetomidine, and followed by administration of sevoflurane.

In some embodiments, ketamine is administered about 1, 2, 3, 4, 5, 10, 15, 20, 25, 30, 35, 40, 45, 50, 55, or 60 minutes prior to, and preferably about 10 to 15 minutes prior to the administration of the pharmaceutical composition. In some embodiments, ketamine is administered at 100 mg/kg, about 90 mg/kg, about 80 mg/kg, about 70 mg/kg, about 60 mg/kg, about 50 mg/k, about 40 mg/kg, about 30 mg/kg, about 20 mg/kg, about 10 mg/kg, about 9 mg/kg, about 8 mg/kg, about 7 mg/kg, about 6 mg/kg, about 5 mg/kg, about 4 mg/kg, about 3 mg/kg, about 2 mg/kg, about 1 mg/kg, preferable at about 10 mg/kg. In some embodiments, The method of any one of claims 90-92, wherein dexmedetomidine is administered at at about 1 mg/kg, about 0.9 mg/kg, about 0.8 mg/kg, about 0.7 mg/kg, about 0.6 mg/kg, about 0.5 mg/k, about 0.4 mg/kg, about 0.3 mg/kg, about 0.2 mg/kg, about 0.1 mg/kg, about 0.09 mg/kg, about 0.08 mg/kg, about 0.07 mg/kg, about 0.06 mg/kg, about 0.05 mg/kg, about 0.04 mg/kg, about 0.03 mg/kg, about 0.02 mg/kg, about 0.01 mg/kg, about 0.009 mg/kg, about 0.008 mg/kg, about 0.007 mg/kg, about 0.006 mg/kg about 0.005 mg/kg, and preferable at about 0.02 mg/kg. In some embodiments, the subject is additionally administered sevolurane following the administration of dexmedetomidine. In some embodiments, sevolurane is administered as an inhalant.

In some preferred embodiments, the subject is administered ketamine (10 mg/kg) 10 to 15 prior to dosing followed by dexmedetomidine (0.02 mg/kg). The subject is then maintained in dorsal recumbence with hind limbs elevated (Trendelenburg like position) for 10 to 15 minutes after completion of adminstration of ketamine and dexmedetomidine. The subject is additionally administered administered atipamezole (0.2 mg/kg IM), with dosing occurs at a standard time 8-10 AM.

In some preferred embodiments, the subject is administered with ketamine (10 mg/kg) 10 to 15 minutes prior to dosing followed by dexmedetomidine (0.02 mg/kg) followed by sevoflurane inhalant anesthesia.

In some embodiments, the agent induces plasma hypertonicity. In some embodiments, the agent comprises hypertonic saline or mannitol. In some embodiments, the agent comprises hypertonic saline. In some embodiments, the hypertonic saline is 3% NaCl. In some embodiments, the 3% NaCl is administered at about 2-3.5 ml/kg.

In some embodiments, the agent is administered by intravenous or infusion injection about 1, 2, 3, 4, 5, 6, 7, 8, 9, 10, 15, 20, preferably about 5 minutes prior or after to administration of the pharmaceutical composition, and optionally wherein the administration can be repeated.

In some embodiments, wherein the agent enhances glymphatic influx by increasing slow wave sleep. In some embodiments, the agent is selected from the group consisting of: Tiagabine, Gaboxadol, Gabapentin, Pregabalin, GHB, Ritanserin, Eplivanserin, Mirtazapine, Olanzapine, and Trazodone, or a combination thereof.

In some embodiments, the agent comprises VEGF, such as VEGF-C. In some embodiments, the VEGF-C comprises: (i) an amino acid sequence of any one of the sequences provided in Table 1 or a sequence with at least 95% sequence identity thereto, optionally wherein the sequence comprises or does not comprise a linker (e.g., a glycine-serine linker) and/or a his tag; and/or (ii) the amino acid substitution of C137A, numbered according to SEQ ID NO: 1.

In some embodiments, the subject is maintained in a position with hind limbs elevated, e.g. Trendelenburg like position, for about 1-2 hours after the administration of the pharmaceutical composition.

In some embodiments, the pharmaceutical composition comprises viral vectors, antibody, antisense oligonucleotide, or nanoparticles.

In some embodiments, the pharmaceutical composition comprises adeno-associated virus (AAV) viral vectors.

In some embodiments, the AAV viral vector comprise a capsid protein derived from AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV 12, AAV16.3, AAV24.1, AAV27.3, AAV42.12, AAV42-lb, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV-17/rh.48, AAV-18/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r 11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAV A3.3, AAV A3.4, AAV A3.5, AAV A3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC 12, AAV-2-pre-miRNA-1O1, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPEN AAV 10 and/or Japanese AAV 10 serotypes, and variants thereof.

In some embodiments, the AAV viral vector comprise a capsid protein derived from AAV9.

In some embodiments, the AAV viral vector comprises a polynucleotide encoding a survival motor neuron (SMN) protein.

In some embodiments, the AAV viral vector comprises a polynucleotide encoding a methyl-CpG-binding protein 2 (MECP2) protein.

In some embodiments, the AAV viral vector comprises a polynucleotide encoding a short hairpin RNA (shRNA) targeting superoxide dismutase 1 (SOD1).

In some embodiments, the AAV viral vector comprises two ITRs (e.g. a modified AAV2 ITR and an unmodified AAV2 ITR), a promoter (e.g. a chicken beta-actin (CB) promoter), an enhancer (e.g. a cytomegalovirus (CMV) immediate/early enhancer), an intro (e.g. a modified SV40 late 16s intron), a polyadenylation signal (e.g. a bovine growth hormone (BGH) polyadenylation signal).

In some embodiments, the pharmaceutical composition comprises between 1×10¹⁰ and 1×10¹⁵ viral vector genomes, such as 1×10¹², 5×10¹², 1×10¹³, 5×10¹³, 1×10¹⁴, 5×10¹⁴, 1×10¹⁵ viral vector genomes.

In some embodiments, the composition comprises between 1×10¹⁰ to 1×10¹⁵ vector genome per milliliter (vg/ml), such as 1×10¹², 5×10¹², 1×10¹³, 5×10¹³, 1×10¹⁴, 5×10¹⁴, 1×10¹⁵ vector genome per milliliter (vg/ml).

In another aspect, the present disclosure provides methods of reducing variable brain distribution of viral vectors among a population of patients treated with a pharmaceutical composition comprising the viral vectors, the method comprising administering to the subject an agent that enhances glymphatic influx in combination with the pharmaceutical composition.

In some embodiments, the agent is administered concurrently or sequentially with the composition. In some embodiments, the agent is administered prior to the administration of the composition. In some embodiments, the agent is administered after the administration of the composition.

In some embodiments, the pharmaceutical composition is administered by intrathecal (IT) administration and/or by intra-cisterna magna (ICM). In some embodiments, the agent is administered by intravenous infusion, intravenous injection and/or inhalation.

In some embodiments, the agent comprises an AQP4 facilitator, e.g. TGN-073.

In some embodiments, the agent comprises a compound that upregulates AQP4, e.g. sevoflurane.

In some embodiments, the agent comprises an α-2 adrenergic agonist, e.g. clonidine, cizanidine, or dexmedetomidine (e.g. Precedex or Dexdomitor).

In some embodiments, the agent comprises one or more FDA approved anesthetics that enhance glymphatic influx. In some embodiments, the anesthetic is ketamine, dexmedetomidine, orxylazine, or a combination thereof.

In preferred embodiments, the agent comprises a combination of ketamine and dexmedetomidine. In some embodiments, the subject is administered first with ketamine, followed by administration of the pharmaceutical composition, and followed by administration of dexmedetomidine. In some embodiments, the subject is administered first with ketamine, followed by administration of the pharmaceutical composition, followed by administration of dexmedetomidine, and followed by administration of sevoflurane.

In some embodiments, ketamine is administered about 1, 2, 3, 4, 5, 10, 15, 20, 25, 30, 35, 40, 45, 50, 55, or 60 minutes prior to, and preferably about 10 to 15 minutes prior to the administration of the pharmaceutical composition. In some embodiments, ketamine is administered about 100 mg/kg, about 90 mg/kg, about 80 mg/kg, about 70 mg/kg, about 60 mg/kg, about 50 mg/k, about 40 mg/kg, about 30 mg/kg, about 20 mg/kg, about 10 mg/kg, about 9 mg/kg, about 8 mg/kg, about 7 mg/kg, about 6 mg/kg, about 5 mg/kg, about 4 mg/kg, about 3 mg/kg, about 2 mg/kg, about 1 mg/kg, preferable at about 10 mg/kg. In some embodiments, dexmedetomidine is administered at about 1 mg/kg, about 0.9 mg/kg, about 0.8 mg/kg, about 0.7 mg/kg, about 0.6 mg/kg, about 0.5 mg/k, about 0.4 mg/kg, about 0.3 mg/kg, about 0.2 mg/kg, about 0.1 mg/kg, about 0.09 mg/kg, about 0.08 mg/kg, about 0.07 mg/kg, about 0.06 mg/kg, about 0.05 mg/kg, about 0.04 mg/kg, about 0.03 mg/kg, about 0.02 mg/kg, about 0.01 mg/kg, about 0.009 mg/kg, about 0.008 mg/kg, about 0.007 mg/kg, about 0.006 mg/kg about 0.005 mg/kg, and preferable at about 0.02 mg/kg. In some embodiments, the subject is additionally administered sevolurane following the administration of dexmedetomidine. In some embodiments, sevolurane is administered as an inhalant.

In some preferred embodiments, the subject is administered ketamine (10 mg/kg) 10 to 15 prior to dosing followed by dexmedetomidine (0.02 mg/kg). The subject is then maintained in dorsal recumbence with hind limbs elevated (Trendelenburg like position) for 10 to 15 minutes after completion of adminstration of ketamine and dexmedetomidine. The subject is additionally administered administered atipamezole (0.2 mg/kg IM), with dosing occurs at a standard time 8-10 AM.

In some preferred embodiments, the subject is administered with ketamine (10 mg/kg) 10 to 15 minutes prior to dosing followed by dexmedetomidine (0.02 mg/kg) followed by sevoflurane inhalant anesthesia.

In some embodiments, the agent induces plasma hypertonicity. In some embodiments, the agent comprises hypertonic saline or mannitol. In some embodiments, the agent comprises hypertonic saline. In some embodiments, the hypertonic saline is 2% NaCl, 3% NaCl, 5% NaCl, 7% NaCl or 23% NaCl, and preferably 3% NaCl. In some embodiments, the 3% NaCl is administered at about 2-3.5 ml/kg.

In some embodiments, the agent is administered by intravenous or infusion injection about 1, 2, 3, 4, 5, 6, 7, 8, 9, 10, 15, 20, preferably about 5 minutes prior or after to administration of the pharmaceutical composition, and optionally wherein the administration can be repeated.

In some embodiments, the agent enhances glymphatic influx by increasing slow wave sleep. In some embodiments, the agent is selected from the group consisting of: Tiagabine, Gaboxadol, Gabapentin, Pregabalin, GHB, Ritanserin, Eplivanserin, Mirtazapine, Olanzapine, and Trazodone, or a combination thereof.

In some embodiments, the agent comprises VEGF, such as VEGF-C. In some embodiments, the VEGF-C comprises: (i) an amino acid sequence of any one of the sequences provided in Table 1 or a sequence with at least 95% sequence identity thereto, optionally wherein the sequence comprises or does not comprise a linker (e.g., a glycine-serine linker) and/or a his tag; and/or (ii) the amino acid substitution of C137A, numbered according to SEQ ID NO: 1.

In some embodiments, the subject is maintained in a position with hind limbs elevated, e.g. Trendelenburg like position, for about 1-2 hours after the administration of the pharmaceutical composition.

In some embodiments, the pharmaceutical composition comprises adeno-associated virus (AAV) viral vectors.

In some embodiments, the AAV viral vector comprise a capsid protein derived from AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV 12, AAV16.3, AAV24.1, AAV27.3, AAV42.12, AAV42-lb, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV-17/rh.48, AAV-18/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r 11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAV A3.3, AAV A3.4, AAV A3.5, AAV A3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC 12, AAV-2-pre-miRNA-1O1, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPEN AAV 10 and/or Japanese AAV 10 serotypes, and variants thereof.

In some embodiments, the AAV viral vector comprise a capsid protein derived from AAV9.

In some embodiments, the AAV viral vector comprises a polynucleotide encoding a survival motor neuron (SMN) protein.

In some embodiments, the AAV viral vector comprises a polynucleotide encoding a methyl-CpG-binding protein 2 (MECP2) protein.

In some embodiments, the AAV viral vector comprises a polynucleotide encoding a short hairpin RNA (shRNA) targeting superoxide dismutase 1 (SOD1).

In some embodiments, the AAV viral vector comprises two ITRs (e.g. a modified AAV2 ITR and an unmodified AAV2 ITR), a promoter (e.g. a chicken beta-actin (CB) promoter), an enhancer (e.g. a cytomegalovirus (CMV) immediate/early enhancer), an intro (e.g. a modified SV40 late 16s intron), a polyadenylation signal (e.g. a bovine growth hormone (BGH) polyadenylation signal).

In some embodiments, the pharmaceutical composition comprises between 1×10¹⁰ and 1×10¹⁵ viral vector genomes, such as 1×10¹², 5×10¹², 1×10¹³, 5×10¹³, 1×10¹⁴, 5×10¹⁴, 1×10¹⁵ viral vector genomes.

In some embodiments, the composition comprises between 1×10¹⁰ to 1×10¹⁵ vector genome per milliliter (vg/ml), such as 1×10¹², 5×10¹², 1×10¹³, 5×10¹³, 1×10¹⁴, 5×10¹⁴, 1×10¹⁵ vector genome per milliliter (vg/ml).

In another aspect, the present disclosure provides methods of reducing systemic exposure of a pharmaceutical composition that targets CNS of a subject in need thereof in order to reduce liver and/or DRG toxicity in the subject, the method comprising administering to the subject an agent that enhances glymphatic influx in combination with the pharmaceutical composition.

In some embodiments, the agent is administered concurrently or sequentially with the composition. In some embodiments, the agent is administered prior to the administration of the composition. In some embodiments, the agent is administered after the administration of the composition.

In some embodiments, the pharmaceutical composition is administered by intrathecal (IT) administration and/or by intra-cisterna magna (ICM).

In some embodiments, the agent is administered by intravenous infusion, intravenous injection and/or inhalation.

In some embodiments, the agent comprises an AQP4 facilitator, e.g. TGN-073.

In some embodiments, the agent comprises a compound that upregulates AQP4, e.g. sevoflurane.

In some embodiments, the agent comprises an α-2 adrenergic agonist, e.g. clonidine, cizanidine, or dexmedetomidine (e.g. Precedex or Dexdomitor).

In some embodiments, the agent comprises one or more FDA approved anesthetics that enhance glymphatic influx. In some embodiments, the anesthetic is ketamine, dexmedetomidine, orxylazine, or a combination thereof.

In preferred embodiments, the agent comprises a combination of ketamine and dexmedetomidine. In some embodiments, the subject is administered first with ketamine, followed by administration of the pharmaceutical composition, and followed by administration of dexmedetomidine. In some embodiments, the subject is administered first with ketamine, followed by administration of the pharmaceutical composition, followed by administration of dexmedetomidine, and followed by administration of sevoflurane.

In some embodiments, ketamine is administered about 1, 2, 3, 4, 5, 10, 15, 20, 25, 30, 35, 40, 45, 50, 55, or 60 minutes prior to, and preferably about 10 to 15 minutes prior to the administration of the pharmaceutical composition. In some embodiments, ketamine is administered at about 100 mg/kg, about 90 mg/kg, about 80 mg/kg, about 70 mg/kg, about 60 mg/kg, about 50 mg/k, about 40 mg/kg, about 30 mg/kg, about 20 mg/kg, about 10 mg/kg, about 9 mg/kg, about 8 mg/kg, about 7 mg/kg, about 6 mg/kg, about 5 mg/kg, about 4 mg/kg, about 3 mg/kg, about 2 mg/kg, about 1 mg/kg, preferable at about 10 mg/kg. In some embodiments, dexmedetomidine is administered at about 1 mg/kg, about 0.9 mg/kg, about 0.8 mg/kg, about 0.7 mg/kg, about 0.6 mg/kg, about 0.5 mg/k, about 0.4 mg/kg, about 0.3 mg/kg, about 0.2 mg/kg, about 0.1 mg/kg, about 0.09 mg/kg, about 0.08 mg/kg, about 0.07 mg/kg, about 0.06 mg/kg, about 0.05 mg/kg, about 0.04 mg/kg, about 0.03 mg/kg, about 0.02 mg/kg, about 0.01 mg/kg, about 0.009 mg/kg, about 0.008 mg/kg, about 0.007 mg/kg, about 0.006 mg/kg about 0.005 mg/kg, and preferable at about 0.02 mg/kg. In some embodiments, the subject is additionally administered sevolurane following the administration of dexmedetomidine. In some embodiments, sevolurane is administered as an inhalant.

In some preferred embodiments, the subject is administered ketamine (10 mg/kg) 10 to 15 prior to dosing followed by dexmedetomidine (0.02 mg/kg). The subject is then maintained in dorsal recumbence with hind limbs elevated (Trendelenburg like position) for 10 to 15 minutes after completion of adminstration of ketamine and dexmedetomidine. The subject is additionally administered administered atipamezole (0.2 mg/kg IM), with dosing occurs at a standard time 8-10 AM.

In some preferred embodiments, the subject is administered with ketamine (10 mg/kg) 10 to 15 minutes prior to dosing followed by dexmedetomidine (0.02 mg/kg) followed by sevoflurane inhalant anesthesia.

In some embodiments, the agent induces plasma hypertonicity. In some embodiments, the agent comprises hypertonic saline (e.g., Sodium chloride with or without sodium acetate) or mannitol. In some embodiments, the agent comprises hypertonic saline with or without sodium acetate. In some embodiments, the hypertonic saline is 2% NaCl, 3% NaCl, 5% NaCl, 7% NaCl or 23% NaCl, and preferably 3% NaCl. In some embodiments, the 3% NaCl is administered at about 2-3.5 ml/kg.

In some embodiments, the agent is administered by intravenous or infusion injection about 1, 2, 3, 4, 5, 6, 7, 8, 9, 10, 15, 20, preferably about 5 minutes prior or after to administration of the pharmaceutical composition, and optionally wherein the administration can be repeated.

In some embodiments, the agent enhances glymphatic influx by increasing slow wave sleep. In some embodiments, the agent is selected from the group consisting of: Tiagabine, Gaboxadol, Gabapentin, Pregabalin, GHB, Ritanserin, Eplivanserin, Mirtazapine, Olanzapine, and Trazodone, or a combination thereof.

In some embodiments, the agent comprises VEGF, such as VEGF-C. In some embodiments, the VEGF-C comprises: (i) an amino acid sequence of any one of the sequences provided in Table 1 or a sequence with at least 95% sequence identity thereto, optionally wherein the sequence comprises or does not comprise a linker (e.g., a glycine-serine linker) and/or a his tag; and/or (ii) the amino acid substitution of C137A, numbered according to SEQ ID NO: 1.

In some embodiments, the subject is maintained in a position with hind limbs elevated, e.g. Trendelenburg like position, for about 1-2 hours after the administration of the pharmaceutical composition.

In some embodiments, the pharmaceutical composition comprises viral vectors, antibody, antisense oligonucleotide, or nanoparticles.

In some embodiments, the pharmaceutical composition comprises adeno-associated virus (AAV) viral vectors.

In some embodiments, the AAV viral vector comprise a capsid protein derived from AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV 12, AAV16.3, AAV24.1, AAV27.3, AAV42.12, AAV42-lb, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV-17/rh.48, AAV-18/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r 11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAV A3.3, AAV A3.4, AAV A3.5, AAV A3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC 12, AAV-2-pre-miRNA-1O1, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPEN AAV 10 and/or Japanese AAV 10 serotypes, and variants thereof.

In some embodiments, the AAV viral vector comprise a capsid protein derived from AAV9.

In some embodiments, the AAV viral vector comprises a polynucleotide encoding a survival motor neuron (SMN) protein.

In some embodiments, the AAV viral vector comprises a polynucleotide encoding a methyl-CpG-binding protein 2 (MECP2) protein.

In some embodiments, the AAV viral vector comprises a polynucleotide encoding a short hairpin RNA (shRNA) targeting superoxide dismutase 1 (SOD1).

In some embodiments, the vector comprises two ITRs (e.g. a modified AAV2 ITR and an unmodified AAV2 ITR), a promoter (e.g. a chicken beta-actin (CB) promoter), an enhancer (e.g. a cytomegalovirus (CMV) immediate/early enhancer), an intro (e.g. a modified SV40 late 16s intron), a polyadenylation signal (e.g. a bovine growth hormone (BGH) polyadenylation signal).

In some embodiments, the pharmaceutical composition comprises between 1×10¹⁰ and 1×10¹⁵ viral vector genomes, such as 1×10¹², 5×10¹², 1×10¹³, 5×10¹³, 1×10¹⁴, 5×10¹⁴, 1×10¹⁵ viral vector genomes.

In some embodiments, the composition comprises between 1×10¹⁰ to 1×10¹⁵ vector genome per milliliter (vg/ml), such as 1×10¹², 5×10¹², 1×10¹³, 5×10¹³, 1×10¹⁴, 5×10¹⁴, 1×10¹⁵ vector genome per milliliter (vg/ml).

Additional embodiments of the invention are provided in the following sections.

### BRIEF DESCRIPTION OF THE DRAWINGS

FIGS. 1A-G are images showing the immunohistochemistry for GFP protein expression in brain following intrathecal dosing by lumbar puncture of 3.0×1013 vg of scAAV9-CB-GFP at 1 month. FIG. 1A is an image of Animal P0304 block 44. FIG. 1B is an image of Animal P0303 block 46. FIG. 1C is an image of Animal P0304 block 44. FIG. 1D is an image of Animal P0302 block 47. FIG. 1E is an image of Animal P0503 bock 47. FIG. 1F is an image of Animal P0303 bock 47. FIG. 1G is an image of Animal P0301 block 51. a, b, c, d and e labels represent enlarged regions boxed in the lower power photomicrograph.

FIG. 2 is am image showing the immunohistochemistry for glial fibrillary acid protein (GFAP) and GFP demonstrating co-localization. GFP positive cells are morphologically consistent with astrocytes (GFP-DAB, left) and co-localize with GFAP (GFAP-blue and GFP-yellow, right).

FIGS. 3A and 3B are, respectively, an image and a scatter plot, showing the quantitative image analysis of GFP expression in spinal cord, dorsal root ganglion and brain regions expressed as percent DAB positive pixels. Moderate to high expression is detected in the lower motor neurons of the spinal cord and neurons of the dorsal root ganglion while minimal and variable expression is detected in multiple regions of the brain.

FIG. 4 is an image showing the immunohistochemistry for GFP protein on sections of cerebellum and brain stem. Minimal transduction of Purkinje cell neurons and neurons within the deep cerebellar nuclei is observed. GFP signal is present primarily with Bergman glia.

FIG. 5 is an image showing the immunohistochemistry for GFP protein. Periventricular GFP protein expression is observed in astrocytes. This periventricular expression was variable in individual animals and limited to the adjacent 500-1000 um of surrounding neuropil.

FIG. 6 an image showing the immunohistochemistry for GFP protein on section of occipital cortex. Multifocal protein expression is present within perivascular astrocytes. a, b and c labels represent enlarged regions boxed in the lower power photomicrograph.

FIG. 7 an image showing the immunohistochemistry for GFP protein on occipital cortex. Expression in perivascular astrocytes adjacent to penetrating artery in occipital cortex is observed consistent with exposure to the vector through glymphatic infux.

FIG. 8 an image showing the detection of GFP protein using immunohistochemistry and quantitative image analysis on occipital cortex. Linear patterns of astrocytic expression adjacent to the arterial vascular supply is observed consistent with exposure to the vector through glymphatic infux.

FIG. 9 an image showing the model of vector distribution to the central nervous system and systemic tissues following intrathecal dosing of AAV in cynomolgus macaque. Due to the rapid turnover of CSF the majority of vector drains from the intrathecal space through arachnoid granulations and nerve roots distributing to systemic tissues. Limited vector reaches brain tissue through glymphatic influx and periventricular diffusion. Approximately 0.01% of the intrathecally administered dose is detected in brain tissue at 30 days compared 1.3% in the liver.

### DETAILED DESCRIPTION

In order that the present disclosure may be more readily understood, certain terms are defined throughout the detailed description. Unless defined otherwise, all technical and scientific terms used herein have the same meaning as commonly understood by those of ordinary skill in the art to which this disclosure pertains.

The disclosed compositions and methods may be understood more readily by reference to the following detailed description taken in connection with the accompanying figures, which form a part of this disclosure.

Throughout this text, the descriptions refer to compositions and methods of using the compositions. Where the disclosure discloses or claims a feature or embodiment associated with a composition, such a feature or embodiment is equally applicable to the methods of using, or uses of the composition. Likewise, where the disclosure discloses or claims a feature or embodiment associated with a method of using a composition, such a feature or embodiment is equally applicable to the composition. When a range of values is expressed, it includes embodiments using any particular value within the range. Further, reference to values stated in ranges includes each and every value within that range. All ranges are inclusive of their endpoints and combinable. When values are expressed as approximations, by use of the antecedent “about,” it will be understood that the particular value forms another embodiment. Reference to a particular numerical value includes at least that particular value, unless the context clearly dictates otherwise. The use of “or” will mean “and/or” unless the specific context of its use dictates otherwise. All references cited herein are incorporated by reference for any purpose. Where a reference and the specification conflict, the specification will control. It is to be appreciated that certain features of the disclosed compositions and methods, which are, for clarity, disclosed herein in the context of separate embodiments, may also be provided in combination in a single embodiment. Conversely, various features of the disclosed compositions and methods that are, for brevity, disclosed in the context of a single embodiment, may also be provided separately or in any sub-combination.

### Definitions

Unless stated otherwise, the following terms and phrases as used herein are intended to have the following meanings:

As used herein, the singular forms “a,” “an,” and “the” include plural forms unless the context clearly dictates otherwise. The term “about” or “approximately,” when used in the context of numerical values and ranges, refers to values or ranges that approximate or are close to the recited values or ranges such that the embodiment may perform as intended, as is apparent to the skilled person from the teachings contained herein. In some embodiments, about means plus or minus 10% of a numerical amount.

As used herein, the term “approximately” or “about,” as applied to one or more values of interest, refers to a value that is similar to a stated reference value. In certain embodiments, the term “approximately” or “about” refers to a range of values that fall within 25%, 20%, 19%,18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the term “glymphatic” or or “glial lymphatic” “glymphatic system” or “glymphatic pathway” refer to a brain waster clearance pathway for the central nervous system (“CNS”) via a perivascular cerebrospinal fluid (CSF) flow pathway. The glymphatic system relies on the interchange of CSF and interstitial fluid (ISF) that allows waste to be transferred to the CSF and transported out of the brain.

As used herein, the term “pharmaceutical composition” means a composition in which the biological activity of the active ingredients has a therapeutic effect, thus the composition can be administered in a subject, e.g. a human, for therpaeutic purposes. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “therapeutic agent” refers to any pharmacologically active substance capable of being administered which achieves a desired effect.

As used herein “neurodegenerative disorder” or “neurodegenerative disease” refers to a central nervous system (CNS) disorder that is characterized by the death of neurons in one or more regions of the nervous system and the subsequent functional impairment of the affected parties. In some embodiments, the neurological disorders may be neurodegenerative disorders including, but not limited to, Alzheimer's Diseases (AD); Amyotrophic lateral sclerosis (ALS); Creutzfeldt-Jakob Disease; Huntingtin's disease (HD); Friedreich's ataxia (FA); Parkinson Disease (PD); Multiple System Atrophy (MSA); Spinal Muscular Atrophy (SMA), Multiple Sclerosis (MS); Primary progressive aphasia; Progressive supranuclear palsy; Dementia; Brain Cancer, Degenerative Nerve Diseases, Encephalitis, Epilepsy, Genetic Brain Disorders that cause neurodegeneration, Retinitis pigmentosa (RP), Head and Brain Malformations, Hydrocephalus, Stroke, Prion disease, Infantile neuronal ceroid lipofuscinosis (INCL) (a neurodegenerative disease of children caused by a deficiency in the lysosomal enzyme palmitoyl protein thioesterase-1 (PPT1)), and others.

As used herein,“treatment” (and grammatical variations thereof such as“treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the disclosure are used to delay development of a disease or to slow the progression of a disease.

An“effective amount” of an agent, e.g., a pharmaceutical formulation, refers to an amount effective, at dosages and for periods of time necessary, to achieve the desired therapeutic or prophylactic result.

An“individual” or“subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

As used herein, the term “intrathecal (IT) administration” or “intrathecal (IT) injection” refers to an injection into the spinal canal (intrathecal space surrounding the spinal cord). Various techniques may be used including, without limitation, lateral cerebroventricular injection through a burrhole or cisternal or lumbar puncture or the like. In some embodiments, “intrathecal administration” or “intrathecal delivery” according to the present disclosure refers to IT administration or delivery via the lumbar area or region, i.e., lumbar IT administration or delivery. As used herein, the term “lumbar region” or “lumbar area” refers to the area between the third and fourth lumbar (lower back) vertebrae and, more inclusively, the L2-S region of the spine.

As used herein, the term “intra-cisterna magna (ICM) administration” or “intra-cisterna magna (ICM) injection” refers to an injection into the space around and below the cerebellum via the opening between the skull and the top of the spine.

As used herein, the term “intracerebroventricular (ICV) administration” or “intracerebroventricular (ICV) injection” refers to an injection into the cavities in the brain that are continuous with the central canal of the spinal cord.

As used herein, the term “AQP4” or “Aquaporin 4” refers to a membrane protein that functions as a water transporter in the central nervous system. It is concentrated in the the perivascular endfeet of astroglial cells that surround blood vessels and maintain the integrity of the blood-brain barrier, and has an important role in the regulation of brain water balance.

As used herein, the term “α-2 adrenergic agonists” refers to chemical entities, usch as compounds, ions, complexes and the like, which are effective to act on or bind to α-2 adrenergic receptors and provide a therapeutic effect.

As used herein, “hypertonic” and “hypotonic” are relative terms e.g., in relation to physiological osmolality, but can diverge from this so long as the ultimate goal of an osmotic differential or gradient is achieved between two compartments (such as the blood plasma and the central nervous system interstitium) so as to promote the influx of glymphatic flow into central nervous system interstitium, brain interstitium and/or a spinal cord interstitium. Accordingly, a“hypertonic solution” refers any physiologically and/or pharmaceutically acceptable solution that is hypertonic with respect to physiological osmolality, including hypertonic saline or sugar solutions. As mentioned herein, hypertonic solutions preferred in this disclosure preferably do not cause BBB disruption.

As used herein, the term “slow wave sleep” refers to phase 3 sleep or deep sleep, which is the deepest phase of non-rapid eye movement (NREM) sleep, and is characterized by delta waives (measured by EEG).

As used herein, the term “VEGF-C” refers to Vascular Endothelial Growth Factor C, which is a member of the platelet-derived growth factor/vascular endothelial growth factor family. VEGF-C is described in detail in WO98/33917, Joukov et al., J. Biol. Chem., 273(12):6599-6602 (1998); and in Joukov et al., EMBO J., 16(13):3898-3911 (1997), all of which are incorporated herein by reference in the entirety.

As used herein the term “antibody” refers to a polypeptide of the immunoglobulin family that is capable of binding a corresponding antigen non-covalently, reversibly, and in a specific manner. For example, a naturally occurring IgG antibody is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antibody” includes, but is not limited to, monoclonal antibodies, human antibodies, humanized antibodies, camelid antibodies, chimeric antibodies, and anti-idiotypic (anti-Id) antibodies (including, e.g., anti-Id antibodies to antibodies of the present disclosure). The antibodies can be of any isotype/class (e.g., IgG, IgE, IgM, IgD, IgA and IgY), or subclass (e.g., IgG1, IgG2, IgG3, IgG4, IgA1 and IgA2).

The term “antisense oligonucleotide” refers to a nucleic acid sequence, regardless of length, that is complementary to the coding strand or mRNA of a nucleic acid sequence. Antisense RNA can be introduced to an individual cell, tissue or organanoid. An anti-sense nucleic acid can contain a modified backbone, for example, phosphorothioate, phosphorodithioate, or other modified backbones known in the art, or may contain non-natural internucleoside linkages.

As used herein, the term “siRNA” intends a double-stranded RNA molecule that interferes with the expression of a specific gene or genes post-transcription. In some embodiments, the siRNA functions to interfere with or inhibit gene expression using the RNA interference pathway. Similar interfering or inhibiting effects may be achieved with one or more of short hairpin RNA (shRNA), micro RNA (mRNA) and/or nucleic acids (such as siRNA, shRNA, or miRNA) comprising one or more modified nucleic acid residue—e.g. peptide nucleic acids (PNA), locked nucleic acids (LNA), unlocked nucleic acids (UNA), or triazole-linked DNA. Optimally, a siRNA is 18, 19, 20, 21, 22, 23 or 24 nucleotides in length and has a 2-base overhang at its 3′ end. These dsRNAs can be introduced to an individual cell or culture system. Such siRNAs are used to downregulate mRNA levels or promoter activity.

As used herein, the term “nanoparticle” refers to any particle having a diameter of less than 1000 nanometers (nm). In some embodiments, the nanoparticles have a diameter of less than 300 nm, as defined by the National Science Foundation. In some embodiments, the nanoparticles have a diameter of less than 100 nm, as defined by the National Institutes of Health. Term “nanoparticle” further include have nano-particles size liposome and Lipid particle.

The terms “polyadenylation (polyA) signal sequence” and “polyadenylation sequence” refer to a regulatory element that provides a signal for transcription termination and addition of an adenosine homopolymeric chain to the 3′ end of an RNA transcript. The polyadenylation signal may comprise a termination signal (e.g., an AAUAAA sequence or other non-canonical sequences) and optionally flanking auxiliary elements (e.g., a GU-rich element) and/or other elements associated with efficient cleavage and polyadenylation. The polyadenylation sequence may comprise a series of adenosines attached by polyadenylation to the 3′ end of an mRNA. Specific polyA signal sequences may include the poly(A) signal of SEQ ID NO:22 or of SEQ ID NO: 89. In some embodiments, DNA regulatory sequences or control elements are tissue-specific regulatory sequences.

The term “post-transcriptional regulatory element” (“PRE”) refers to one or more regulatory elements that, when transcribed into mRNA, regulate gene expression at the level of the mRNA transcript. Examples of such post-transcriptional regulatory elements may include sequences that encode micro-RNA binding sites, RNA binding protein binding sites, etc. Examples of post-transcriptional regulatory element that may be used with the nucleic acid molecules and vectors disclosed herein include the woodchuck hepatitis post-transcriptional regulatory element (WPRE), the hepatitis post-transcriptional regulatory element (HPRE).

The terms “polynucleotide” and “nucleic acid” are used interchangeably herein and refer to a polymeric form of nucleotides of any length. They may include one or more of ribonucleotides or deoxyribonucleotides. Thus, this term includes, but is not limited to, single-double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases, e.g. locked nucleic acids (LNA), peptide nucleic acids (PNA).

The terms “peptide,” “polypeptide,” and “protein” are used interchangeably, and refer to a compound comprised of amino acid residues covalently linked by peptide bonds. A protein or peptide typically contains at least two amino acids or amino acid variants, and no limitation is placed on the maximum number of amino acids that can comprise a protein's or peptide's sequence. Polypeptides include any peptide or protein comprising two or more amino acids or variants joined to each other by peptide bonds. The terms include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “sequence identity” and “sequence homology” are used interchangeably herein, and as used in connection with a polynucleotide or polypeptide, refers to the percentage of bases or amino acids that are the same, and are in the same relative position, when comparing or aligning two sequences of polynucleotides of polypeptides. Sequence identity can be determined in a number of different manners. For instance, sequences may be aligned using various methods and computer programs (e.g., BLAST, T-COFFEE, MUSCLE, MAFFT, etc.). See, e.g., Altschul et al., (1990) J. Mol. Bioi., 215:403-10.

The term “isolated” in reference to a nucleic acid or protein discussed herein refers to a nucleic acid or protein that has been separated from one or more of the components normally found associated with it in the natural environment. The separation may comprise removal from a larger nucleic acid (e.g., from a gene or chromosome) or from other proteins or molecules normally in contact with the nucleic acid or protein. The term encompasses but does not require complete isolation.

As used herein, an isolated nucleic acid comprising a “heterologous nucleic acid sequence” refers to an isolated nucleic acid comprising a portion (i.e., the heterologous nucleic acid portion) that is not normally found operably linked to one or more other components of the isolated nucleic acid in a natural context. For instance, the heterologous nucleic acid may comprise a nucleic acid sequence not originally found in a cell, bacterial cell, virus, or organism from which other components of the isolated nucleic acid (e.g., the promoter) naturally derive or where the other components of the isolated nucleic acid (e.g., the promoter) are not naturally found operatively linked with the heterologous nucleic acid in the cell, bacterial cell, virus, or organism. In some embodiments the heterologous nucleic acid includes a transgene. As used herein, a “transgene” is a nucleic acid sequence that encodes a molecule of interest (for example, a therapeutic protein, a reporter protein or a therapeutic RNA molecule) that is not originally associated with one or more components of the nucleic acid molecule. In some embodiments, the heterologous nucleic acid sequence encodes a human protein. In some embodiments, the heterologous nucleic acid sequence encodes an RNA sequence, e.g., a shRNA.

A DNA sequence or DNA polynucleotide sequence that “encodes” a particular RNA is a sequence of DNA that is capable of being transcribed into RNA. A DNA polynucleotide may encode an RNA (mRNA) that is translated into protein, or a DNA polynucleotide may encode an RNA that is not translated into protein (e.g. tRNA, rRNA, or a guide RNA; also called “non-coding” RNA or “ncRNA”). A DNA sequence or DNA polynucleotide sequence may also “encode” a particular polypeptide or protein sequence, wherein, for example, the DNA directly encodes an mRNA that can be translated into the polypeptide or protein sequence. A “protein coding sequence” or a sequence that encodes a particular protein or polypeptide is a nucleic acid sequence that is capable of being transcribed into mRNA (in the case of DNA) and translated (in the case of mRNA) into a polypeptide in vitro or in vivo when placed under the control of appropriate regulatory sequences. The boundaries of the coding sequence may be determined by a start codon at the 5′ terminus (N-terminus) and a translation stop nonsense codon at the 3′ terminus (C-terminus). A coding sequence can include, but is not limited to, cDNA from prokaryotic or eukaryotic mRNA, genomic DNA sequences from prokaryotic or eukaryotic DNA, and synthetic nucleic acids. A transcription termination sequence will usually be located 3′ to the coding sequence.

The term “promoter” or “promoter sequence” as used herein is a DNA regulatory sequence capable of facilitating transcription (e.g., capable of causing detectable levels of transcription and/or increasing the detectable level of transcription over the level provided in the absence of the promoter) of an operatively linked coding or non-coding sequence, e.g., of a downstream (3′ direction) coding or non-coding sequence, e.g., through binding RNA polymerase. In some embodiments, the promoter sequence is bounded at its 3′ terminus by the transcription initiation site and extends upstream (5′ direction) to include the minimum number of bases or elements to initiate transcription at levels detectable above background. In some embodiments, a promoter sequence may comprise a transcription initiation site, as well as protein binding domains responsible for the binding of RNA polymerase. In addition to sequences sufficient to initiate transcription, a promoter may also include sequences of other regulatory elements that are involved in modulating transcription (e.g., enhancers, Kozak sequences and introns). Various promoters, including inducible promoters and constitutive promoters, may be used to drive the vectors disclosed herein. Examples of promoters known in the art that may be used in some embodiments, e.g., in viral vectors disclosed herein, include the CMV promoter, CBA promoter, smCBA promoter and those promoters derived from an immunoglobulin gene, SV40, or other tissue specific genes (e.g: RLBP1, RPE, VMD2). In addition, standard techniques are known in the art for creating functional promoters by mixing and matching known regulatory elements. Fragments of promoters, e.g., those that retain at least minimum number of bases or elements to initiate transcription at levels detectable above background, may also be used.

The terms “DNA regulatory sequences,” “control elements,” and “regulatory elements,” used interchangeably herein, refer to transcriptional and translational control sequences, such as promoters, enhancers, silencers, polyadenylation signals, terminators, protein degradation signals, and the like, that provide for and/or regulate transcription of a non-coding sequence (e.g., a short hairpin RNA) or a coding sequence (e.g., PGRN) and/or regulate translation of an encoded polypeptide.

As used herein, processes conducted “in vitro” refer to processes which are performed outside of the normal biological environment, for example, studies performed in a test tube, a flask, a petri dish, in artificial culture medium. Processes conducted “in vivo” refer to processes performed within living organisms or cells. for example, studies performed in cell cultures or in mice. Studies performed “ex vivo” refer to studies done in or on tissue from an organism in an external environment, e.g., with minimal alteration of natural conditions, e.g., allowing for manipulation of an organism's cells or tissues under more controlled conditions than may be possible in in vivo experiments.

The term “naturally-occurring” or “unmodified” as used herein as applied to, e.g., a nucleic acid, a polypeptide, a cell, or an organism, is one found in nature. For example, a polypeptide or polynucleotide sequence that is present in an organism (such as a virus) is naturally occurring whether present in that organism or isolated from one or more components of the organism.

In some embodiments, a “vector” is any genetic element (e.g., DNA, RNA, or a mixture thereof) that contains a nucleic acid of interest (e.g., a transgene) that is capable of being expressed in a host cell, e.g., a nucleic acid of interest within a larger nucleic acid sequence or structure suitable for delivery to a cell, tissue, and/or organism, such as a plasmid, phage, transposon, cosmid, chromosome, virus, virion, etc. For instance, a vector may comprise an insert (e.g., a heterologous nucleic acid comprising a transgene encoding a gene to be expressed or an open reading frame of that gene) and one or more additional elements, and/or elements suitable for delivering or controlling expression of the insert. The vector may be capable of replication and/or expression, e.g., when associated with the proper control elements, and it may be capable of transferring genetic information between cells. In some embodiments, a vector may be a vector suitable for expression in a host cell, e.g, an AAV vector. In some embodiments, a vector may be a plasmid suitable for expression and/or replication, e.g., in a cell or bioreactor. In some embodiments, vectors designed specifically for the expression of a heterologous nucleic acid sequence, e.g., a transgene encoding a protein of interest, shRNA, and the like, in the target cell may be referred to as expression vectors, and generally have a promoter sequence that drives expression of the transgene. In other embodiments, vectors, e.g., transcription vectors, may be capable of being transcribed but not translated: they can be replicated in a target cell but not expressed. Transcription vectors may be used to amplify their insert.

The term “plasmid” refers to a nonchromosomal (and typically double-stranded) DNA sequence comprising an intact “replicon” such that the plasmid is replicated in a host cell. A plasmid may be a circular nucleic acid. When the plasmid is placed within a unicellular organism, the characteristics of that organism are changed or transformed as a result of the DNA of the plasmid. For example, a plasmid carrying the gene for tetracycline resistance (TcR) transforms a cell previously sensitive to tetracycline into one which is resistant to it.

The term “recombinant virus” as used herein is intended to refer to a non-wild-type and/or artificially produced recombinant virus (e.g., a parvovirus, adenovirus, lentivirus or adeno-associated virus etc.) that comprises a transgene or other heterologous nucleic acid. The recombinant virus may comprise a recombinant viral genome packaged within a viral (e.g.: AAV) capsid. A specific type of recombinant virus may be a “recombinant adeno-associated virus”, or “rAAV”. The recombinant viral genome packaged in the viral capsid may be a viral vector. In some embodiments, the recombinant viruses disclosed herein comprise viral vectors. Examples of viral vectors include but are not limited to an adeno-associated viral (AAV) vector, a chimeric AAV vector, an adenoviral vector, a retroviral vector, a lentiviral vector, a DNA viral vector, a herpes simplex viral vector, a baculoviral vector, or any mutant or derivative thereof.

In another embodiment, the term “transfection” is used to refer to the uptake of foreign DNA by a cell, such that the cell has been “transfected” once the exogenous DNA has been introduced inside the cell membrane. See, e.g., Graham et al., (1973) Virology, 52:456; Sambrook et al., (1989) Molecular Cloning, a laboratory manual, Cold Spring Harbor Laboratories, New York; Davis et al., (1986) Basic Methods in Molecular Biology, Elsevier; Chu et al., (1981) Gene, 13:197. Such techniques can be used to introduce one or more exogenous DNA moieties into suitable host cells. In some embodiments, the term “transduction” is used to refer to the uptake of foreign DNA by a cell, where the foreign DNA is provided by a virus or a viral vector. Consequently, a cell has been “transduced” when exogenous DNA has been introduced inside the cell membrane. In some embodiments, the term “transformation” is used to refer to the uptake of foreign DNA by bacterial cells.

As used herein, the term “cell line” refers to a population of cells capable of continuous or prolonged growth and division in vitro. In certain circumstances, spontaneous or induced changes can occur in karyotype during storage or transfer of such clonal populations. Therefore, cells derived from the cell line referred to may not be precisely identical to the ancestral cells or cultures, and the cell line referred to includes such variants.

The term “operably linked” refers to a functional relationship between two or more polynucleotide (e.g., DNA) segments. Typically, the term refers to the functional relationship of a transcriptional regulatory sequence and a sequence to be transcribed. For example, a promoter or enhancer sequence is operably linked to a coding sequence if it, e.g., stimulates or modulates the transcription of the coding sequence in an appropriate host cell or other expression system. Generally, promoter transcriptional regulatory sequences that are operably linked to a sequence are contiguous to that sequence or are separated by short spacer sequences, i.e., they are cis-acting. However, some transcriptional regulatory sequences, such as enhancers, need not be physically contiguous or located in close proximity to the coding sequences whose transcription they enhance.

As used herein, the term “AAV vector” refers to a vector derived from or comprising one or more nucleic acid sequences derived from an adeno-associated virus serotype, including without limitation, an AAV-1, AAV-2, AAV-3, AAV-4, AAV-5, AAV-6, AAV-7, AAV-8 or AAV-9 viral vector. AAV vectors may have one or more of the AAV wild-type genes deleted in whole or part, e.g., the rep and/or cap genes, while retaining, e.g., functional flanking inverted terminal repeat (“ITR”) sequences. In some embodiments, an AAV vector may be packaged in a protein shell or capsid, e.g., comprising one or more AAV capsid proteins, which may provide a vehicle for delivery of vector nucleic acid to the nucleus of target cells. In some embodiments, an AAV vector comprises one or more AAV ITR sequences (e.g., AAV2 ITR sequences). In some embodiments, an AAV vector comprises one or more AAV ITR sequences (e.g., AAV2 ITR sequences) but does not contain any additional viral nucleic acid sequence. In some embodiments, the AAV vector components (e.g., ITRs) are derived from a different serotype virus than the rAAV capsid (for example, the AAV vector may comprise ITRs derived from AAV2 and the AAV vector may be packaged into an AAV9 capsid). Embodiments of these vector constructs are provided, e.g., in WO/2019/094253 (PCT/US2018/058744), which is incorporated herein by reference in its entirety.

In some embodiments, an “scAAV” is a self-complementary adeno-associated virus (scAAV). scAAV is termed “self-complementary” because at least a portion of the vector (e.g., at least a portion of the coding region) of the scAAV forms an intra-molecular double-stranded DNA. In some embodiments, the rAAV is an scAAV. In some embodiments, a viral vector is engineered from a naturally occurring adeno-associated virus (AAV) to provide an scAAV for use in gene therapy. Embodiments of these vector constructs and methods of preparing and purifying them are provided, e.g., in WO/2019/094253 (PCT/US2018/058744), which is incorporated herein by reference in its entirety.

In some embodiments, an “ssAAV” is a single-stranded adeno-associated virus (ssAAV). ssAAV is termed “single-stranded” because at least a portion of the vector (e.g., at least a portion of the coding region) of the ssAAV is sigle-stranded DNA. In some embodiments, the rAAV is an ssAAV. In some embodiments, a viral vector is engineered from a naturally occurring adeno-associated virus (AAV) to provide an ssAAV for use in gene therapy.

As used herein, an “virus” or “virion” indicates a viral particle, comprising a viral vector, e.g., alone or in combination with one or more additional components such as one or more viral capsids. For instance, an AAV virus may comprise, e.g., a linear, single-stranded AAV nucleic acid genome associated with an AAV capsid protein coat.

In some embodiments, terms such as “virus,” “virion,” “AAV virus,” “recombinant AAV virion,” “rAAV virion,” “AAV vector particle,” “full capsids,” “full particles,” and the like refer to infectious, replication-defective virus, e.g., those comprising an AAV protein shell encapsidating a heterologous nucleotide sequence of interest, e.g., in a viral vector which is flanked on one or both sides by AAV ITRs. A rAAV virion may be produced in a suitable host cell which comprises sequences, e.g., one or more plasmids, specifying an AAV vector, alone or in combination with nucleic acids encoding AAV helper functions and accessory functions (such as cap genes), e.g., on the same or additional plasmids. In some embodiments, the host cell is rendered capable of encoding AAV polypeptides that provide for packaging the AAV vector (containing a recombinant nucleotide sequence of interest) into infectious recombinant virion particles for subsequent gene delivery.

The terms “inverted terminal repeat” or “ITR” refer to a stretch of nucleotide sequences that can form a T-shaped palindromic structure, e.g., in adeno-associated viruses (AAV) and/or recombinant adeno-associated viral vectors (rAAV). Muzyczka et al., (2001) Fields Virology, Chapter 29, Lippincott Williams & Wilkins. In recombinant AAV vectors, these sequences may play a functional role in genome packaging and in second-strand synthesis.

The term “host cell” denotes a cell comprising an exogenous nucleic acid of interest, for example, one or more microorganism, yeast cell, insect cell, or mammalian cell. For instance, the host cell may comprise an AAV helper construct, an AAV vector plasmid, an accessory function vector, and/or other transfer DNA. The term includes the progeny of the original cell which has been transfected. The progeny of a single parental cell may not necessarily be completely identical in morphology or in genomic or total DNA complement as the original parent, due to natural, accidental, or deliberate mutation.

The term “AAV helper function” refers to an AAV-derived coding sequences which can be expressed to provide AAV gene products, e.g., those that function in trans for productive AAV replication. For instance, AAV helper functions may include both of the major AAV open reading frames (ORFs), rep and cap. The Rep expression products have been shown to possess many functions, including, among others: recognition, binding and nicking of the AAV origin of DNA replication; DNA helicase activity; and modulation of transcription from AAV (or other heterologous) promoters. The Cap expression products supply necessary packaging functions. AAV helper functions may be used herein to complement AAV functions in trans that are missing from AAV vectors.

The term “AAV helper construct” refers generally to a nucleic acid molecule that includes nucleotide sequences providing or encoding proteins or nucleic acids that provide AAV functions deleted from an AAV vector, e.g. a vector for delivery of a nucleotide sequence of interest to a target cell or tissue. AAV helper constructs are commonly used to provide transient expression of AAV rep and/or cap genes to complement missing AAV functions for AAV replication. Typically, helper constructs lack AAV ITRs and can neither replicate nor package themselves. AAV helper constructs may be in the form of a plasmid, phage, transposon, cosmid, virus, or virion. A number of AAV helper constructs have been disclosed, such as the commonly used plasmids pAAV/Ad and plM29+45 which encode both Rep and Cap expression products. See, e.g., Samulski et al., (1989) J. Virol., 63:3822-3828; McCarty et al., (1991) J. Virol., 65:2936-2945. A number of other vectors have been disclosed which encode Rep and/or Cap expression products. See, e.g., U.S. Pat. Nos. 5,139,941 and 6,376,237. Embodiments of these vector constructs and methods of preparing and purifying them are provided, e.g., in WO/2019/094253 (PCT/US2018/058744), which is incorporated herein by reference in its entirety.

### Glymphatic Flow

In one aspect, the present disclosure provides methods for improving AAV delivery of agents to target tissues, e.g. brain, by modulating glymphatic influx. The glymphatics are a recently recognized system by which CSF is drawn into the deeper regions of the brain along periarterial spaces formed by vessel adjacent astrocytes where CSF may exchange with the interstitial fluid prior to exiting the brain in an equivalent perivenule space. This system is thought to play a major role in the movement of fluid and removal macromolecules from the brain parenchyma. Larger particles such as lipoproteins which are of equivalent size to AAV vectors move through the glymphatic system. It is discovered that the AAV distribution patterns are consistent with limited diffusion of vector across membranes lining the brain surface and vector entry occurring primarily through glymphatic influx. It is unexpectly discovered that AAV delivery into the central nervous system interstitium, brain interstitium and/or the spinal cord interstitium can be achieved by enhancing glymphatic influx. Specifically, the present disclosure provides that enhancing glymphatic influx can be used for i) improving delivery of a pharmaceutical composition to the central nervous system of a subject in need thereof; ii) methods of treating a neurological disease to a subject in need thereof, wherein the pharmaceutical composition comprises an AAV encoding a gene associated with the neurological disease; iii) methods of improving the transduction efficiency and/or distribution of a neurodegenerative therapeutic agent in brain; iv) methods of increasing efficacy of an intrathecally delivered pharmaceutical composition; v) methods of reducing variable brain distribution of viral vectors among a population of patients treated with a pharmaceutical composition comprising the viral vectors; and vi) methods of reducing systemic exposure of a pharmaceutical composition that targets CNS of a subject in need thereof in order to reduce liver and/or DRG toxicity in the subject.

Glymphatic influx can be enhanced via a number of ways. In some embodiments, glymphatic influx can be enhanced by timing of vector administration to coincide with CSF influx during sleep cycle. In some embodiments, glymphatic influx can be enhanced by administering a AQP4 facilator, e.g. TGN-073. In some embodiments, glymphatic influx can be enhanced by AQP4 upregulation, e.g. by Sevoflurane anesthesia. In some embodiments, glymphatic influx can be enhanced by α-2 adrenergic agonist, e.g. dexmedetomidine (Precedex or Dexdomitor). In some embodiments, glymphatic influx can be enhanced by a combination of ketamine and xylazine. In some embodiments, glymphatic influx can be enhanced by induction of plasma hypertonicity with hypertonic saline or mannitol.

### AQP4

Aquaporin 4 (AQP4), a water channel subtype, is highly expressed in the brain. The interchange of CSF and ISF is dependent on aquaporin 4 (AQP4) water channels on astrocyte endfeet that enwrap the cerebral vascula-ture. Changes in AQP4 expression or polarisation—referring to the differential distribution of AQP4 in the endfeet versus rest of the cell—are associated with disturbances in glymphatic function. In line with the observation that the glymphatic system can clear amyloid-b, decreased glymphatic function caused by deletion of the Aqp4 gene in an animal model of Alzheimer's disease leads to increased accumulation of amyloid-b2 and tau.16 Abnormalities in AQP4 polar-isation are also seen in Alzheimer's patients, which provides some evidence that glymphatic function might also play a role in Alzheimer's disease in humans. Kylkilahti et al., Journal of Cerebral Blood Flow & Metabolism, 0(0):1-13 (2021).

In some embodiments, the glymphatic influx is enhanced by an agent that promotes interstitial fluid circulation within the blood-brain barrier, e.g., wherein the agent comprises an Aquaporin 4 (AQP4) facilitator, e.g. TGN-073 (N-(3-benzyloxypyridin-2-yl)-benzene-sulfonamide). In some embodiments, the agent comprises a compound that upregulates AQP4 expression (e.g. sevoflurane) or alters subcellular localization of AQP4. In another embodiment, the agent can be an agent that prevents AQP4 depolarization or loss of AQP4 polarization, such as JNJ-1 7299425 or JNJ-17306861.

### α-2 Adrenergic Agonist and Anesthetics

The glymphatic pathway is predominantly active during sleep or anesthesia that promotes slow-wave oscillations. Decreased CNS noradrenergic tone, an important feature of deep NREM sleep, has been associated with high glymphatic influx as it decreases resistance to interstitial fluid flow by enlarging the interstitial space volume. α2-adrenergic agonists are known sedative agents, which induces a sedative state similar to stage II-III NREM sleep with respect to the increased slow-wave delta oscillations in the electroencephalogram (EEG) and dramatically decreased noradrenergic tone. Studies have shown that Dexmedetomidine, a selective α2-adrenergic agonist, by modulating glymphatic flow, enhances EEG slow-wave activity, increases brain and spinal cord drug exposure of intrathecally administered drugs in mice and rats. T. O. Lilius et al., Journal of Controlled Release, 304:29-38 (2019). Glymphatic system activity increases during sleep or ketamine/xylazine (K/X) anesthesia in mice, and is correlated with high EEG delta power and low heart rate. Hablitz et al., Sci Adv. 5(2): eaav5447 (2019).

In an embodiment, the agent is an alpha2-adrenergic receptor (α2-AR) agonist. In an embodiment, the α2-AR agonist is dexmedetomidine. See, e.g., Lilius T O, et al. Dexmedetomidine enhances glymphatic brain delivery of intrathecally administered drugs. J Control Release. 2019 Jun. 28; 304:29-38, which is incorporated by reference in its entirety. In some embodiments, the agent comprises an α-2 adrenergic agonist selected from the group consisting of clonidine, cizanidine, and dexmedetomidine (e.g. Precedex or Dexdomitor).

In an embodiment, the agent enhances glymphatic flow. In an embodiment, the agent enhances glymphatic influx. In an embodiment, the agent is an anesthetic, e.g., a general anesthetic. In an embodiment, the anesthetic is selected from the group consisting of propofol, fospropofol, ketamine, barbiturates (e.g., thiopental, thiopentone, and methohexital), benzodiazepines (e.g., midazolam), etomidate, isoflurane, desflurane, and sevoflurane. See, e.g., Hablitz L M, et al. Increased glymphatic influx is correlated with high EEG delta power and low heart rate in mice under anesthesia. Sci Adv. 2019 Feb. 27; 5(2):eaav5447, which is incorporated by reference in its entirety.

In some embodiments, the agent comprises one or more FDA approved anesthetics that enhance glymphatic influx. In some embodiments, the anesthetic is ketamine, dexmedetomidine, orxylazine, or a combination thereof. In preferred embodiments, the agent comprises a combination of ketamine and dexmedetomidine.

In some embodiments, the subject is administered first with ketamine, followed by administration of the pharmaceutical composition, and followed by administration of dexmedetomidine.

In some embodiments, ketamine is administered about 1, 2, 3, 4, 5, 10, 15, 20, 25, 30, 35, 40, 45, 50, 55, or 60 minutes prior to, and preferably about 10 to 15 minutes prior to the administration of the pharmaceutical composition.

In some embodiments, ketamine is administered at about 100 mg/kg, about 90 mg/kg, about 80 mg/kg, about 70 mg/kg, about 60 mg/kg, about 50 mg/k, about 40 mg/kg, about 30 mg/kg, about 20 mg/kg, about 10 mg/kg, about 9 mg/kg, about 8 mg/kg, about 7 mg/kg, about 6 mg/kg, about 5 mg/kg, about 4 mg/kg, about 3 mg/kg, about 2 mg/kg, about 1 mg/kg, preferable at about 10 mg/kg.

In some embodiments, dexmedetomidine is administered at about 1 mg/kg, about 0.9 mg/kg, about 0.8 mg/kg, about 0.7 mg/kg, about 0.6 mg/kg, about 0.5 mg/k, about 0.4 mg/kg, about 0.3 mg/kg, about 0.2 mg/kg, about 0.1 mg/kg, about 0.09 mg/kg, about 0.08 mg/kg, about 0.07 mg/kg, about 0.06 mg/kg, about 0.05 mg/kg, about 0.04 mg/kg, about 0.03 mg/kg, about 0.02 mg/kg, about 0.01 mg/kg, about 0.009 mg/kg, about 0.008 mg/kg, about 0.007 mg/kg, about 0.006 mg/kg about 0.005 mg/kg, and preferable at about 0.02 mg/kg.

In some embodiments, the subject is additionally administered sevolurane following the administration of dexmedetomidine.

In some embodiments, sevolurane is administered as an inhalant.

### Induce Plasma Hypertonicity

In another aspect, enhancing glymphatic influx can be achieved by inducing plasma hypertonicity. As used herein, “hypertonic” and “hypotonic” are relative terms e.g., in relation to physiological osmolality, but can diverge from this so long as the ultimate goal of an osmotic differential or gradient is achieved between two compartments (such as the blood plasma and the central nervous system interstitium) so as to promote the influx of glymphatic flow into central nervous system interstitium, brain interstitium and/or a spinal cord interstitium. Accordingly, a“hypertonic solution” refers any physiologically and/or pharmaceutically acceptable solution that is hypertonic with respect to physiological osmolality, including hypertonic saline or sugar solutions. As mentioned herein, hypertonic solutions preferred in this disclosure do not cause BBB disruption.

In some embodiments, the agent comprises hypertonic saline (e.g., Sodium chloride with or without sodium acetate) or mannitol. In preferred embodiments, the agent comprises hypertonic saline with or without sodium acetate. In some embodiments, the hypertonic saline is 2% NaCl, 3% NaCl, 5% NaCl, 7% NaCl or 23% NaCl, and preferably 3% NaCl. In some embodiments, the 3% NaCl is administered at about 2-3.5 ml/kg.

In some embodiments, the agent is administered by intravenous or infusion injection about 1, 2, 3, 4, 5, 6, 7, 8, 9, 10, 15, 20, preferably about 5 minutes prior or after to administration of the pharmaceutical composition, and optionally wherein the administration can be repeated.

### Sleep

Studies have shown that glymphatic function is increased during sleep and impaired by sleep disturbances. Xie et al., Science 342:373-377 (2013). Sleep deprivation leads to changes in AQP4 expression. Kylkilahti et al., Journal of Cerebral Blood Flow & Metabolism 00:1-13 (2021).

In an embodiment, the agent is a non-anesthetic agent that increases slow wave sleep. In an embodiment, the agent is selected from the group consisting of a GAT-1 inhibitor, selective extrasynaptic GABAA agonist, α2-δ site on voltage-gated calcium ion channels, GABAB/GHB agonist, partially selective 5HT2A receptor antagonist, and antagonist of serotonin Two A Receptors (ASTAR). In an embodiment, the agent is selected from the group consisting of Tiagabine, Gaboxadol, Gabapentin, Pregabalin, GHB, Ritanserin, Eplivanserin, Mirtazapine, Olanzapine, and Trazodone. Additional agents that increase slow wave sleep are described in Walsh, J. K. Enhancement of Slow Wave Sleep: Implications for Insomnia. Journal of Clinical Sleep Medicine. 2009, 5(2): S27-S32, which is incorporated by reference in its entirety.

### Enhances VEGF-C

Studies show that vascular endothelial growth factor C (VEGF-C) binds a receptor on lymphatic endothelial cells, and also affects vascular endothelim. Joukov et al. EMBO J. 15(2):290-298 (1996). Expression of VEGF-C in mice leads to gowths of lymphatic vessels. Da Mesquita et al., Nature 560(7717):185-191 (2018).

In some embodiments, the agent that enhances glymphatic influx comprises VEGF-C. In some embodiments, the VEGF-C comprises: (i) an amino acid sequence of any one of the sequences provided in Table 1 or a sequence with at least 95% sequence identity thereto, optionally wherein the sequence comprises or does not comprise a linker (e.g., a glycine-serine linker) and/or a his tag; and/or (ii) the amino acid substitution of C137A, numbered according to SEQ ID NO: 1.

### Table 1 VEGF-C and its Variants

The sequences below correspond to a monomer. When a dimer is formed, 2 of the same sequences are assembled together via cysteine bridges. It is noted that the his tag is used for experimental purposes but may not be necessary in all embodiments.

| SEQ | | | |
|---|---|---|---|
| ID | | | |
| NO: | DESCRIPTOR | SEQUENCE | KEY |
| 1 | >sp\|P49767\| | MHLLGFFSVACSLLAAALLPGPREAPAAAAAFE | Underlined: |
| | VEGFC_HUMAN | SGLDLSDAEPDAGEATAYASKDLEEQLRSVSS | cleaved residues |
| | Vascular | VDELMTVLYPEYWKMYKCQLRKGGWQHN | Bold: propeptide |
| | endothelial growth | REQANLNSRTEETIKFAAAHYNTEILKSIDNEW | |
| | factor C | RKTQCMPREVCIDVGKEFGVATNTFFKPPCVSV | |
| | OS = Homo sapiens | YRCGGCCNSEGLQCMNTSTSYLSKTLFEITVPLS | |
| | OX = 9606 | QGPKPVTISFANHTSCRCMSKLDVYRQVHSIIRR | |
| | GN = VEGFC PE = 1 | SLPATLPQCQAANKTCPTNYMWNNHICRCLAQ | |
| | SV = 1 | EDFMFSSDAGDDSTDGFHDICGPNKELDEETCQ | |
| | | CVCRAGLRPASCGPHKELDRNSCQCVCKNKLFP | |
| | | SQCGANREFDENTCQCVCKRTCPRNQPLNPGK | |
| | | CACECTESPQKCLLKGKKFHHQTCSCYRRPCTN | |
| | | RQKACEPGFSYSEEVCRCVPSYWKRPQMS | |
| 2 | >1 immature | FESGLDLSDAEPDAGEATAYASKDLEEQLRSVS | Underlined: |
| | peptide | SVDELMTVLYPEYWKMYKCQLRKGGWQHNRE | propeptide |
| | (32-227)- | QANLNSRTEETIKFAAAHYNTEILKSIDNEWRKT | Double underlined: |
| | VEGFC_His | QCMPREVCIDVGKEFGVATNTFFKPPCVSVYRC | GS linker |
| | | GGCCNSEGLQCMNTSTSYLSKTLFEITVPLSQGP | Bold: his tag |
| | | KPVTISFANHTSCRCMSKLDVYRQVHSIIRRGSH | |
| | | HHHHH | |
| 3 | >1 immature | FESGLDLSDAEPDAGEATAYASKDLEEQLRSVS | Underlined: |
| | peptide | SVDELMTVLYPEYWKMYKCQLRKGGWQHNRE | propeptide |
| | (32-227)-VEGFC | QANLNSRTEETIKFAAAHYNTEILKSIDNEWRKT | |
| | | QCMPREVCIDVGKEFGVATNTFFKPPCVSVYRC | |
| | | GGCCNSEGLQCMNTSTSYLSKTLFEITVPLSQGP | |
| | | KPVTISFANHTSCRCMSKLDVYRQVHSIIRR | |
| 4 | >2 major mature | AHYNTEILKSIDNEWRKTQCMPREVCIDVGKEF | Double underlined: |
| | (112-227)- | GVATNTFFKPPCVSVYRCGGCCNSEGLQCMNT | GS linker |
| | VEGFC_His | STSYLSKTLFEITVPLSQGPKPVTISFANHTSCRC | Bold: his tag |
| | | MSKLDVYRQVHSIIRRGSHHHHHH | |
| 5 | >2 major mature | AHYNTEILKSIDNEWRKTQCMPREVCIDVGKEF | |
| | (112-227)-VEGFC | GVATNTFFKPPCVSVYRCGGCCNSEGLQCMNT | |
| | | STSYLSKTLFEITVPLSQGPKPVTISFANHTSCRC | |
| | | MSKLDVYRQVHSIIRR | |
| 6 | >7 minor mature | TEETIKFAAahynteilksidnewrktqcmprevcidvgkefgvat | Capital: fragment |
| | (103-227)- | ntffkppcvsvyrcggccnseglqcmntstsylsktlfeitvplsqgp | of propeptide |
| | VEGFC His | kpvtisfanhtscrcmskldvyrqvhsiirrgshhhhhh | Double underlined: |
| | | | GS linker |
| | | | Bold: his tag |
| 7 | >7 minor mature | TEETIKFAAahynteilksidnewrktqcmprevcidvgkefgvat | Capital: fragment |
| | (103-227)-VEGFC | ntffkppcvsvyrcggccnseglqcmntstsylsktlfeitvplsqgp | of propeptide |
| | | kpvtisfanhtscrcmskldvyrqvhsiirr | |
| 8 | >8 minor mature | TEETIKFAAahynteilksidnewrktqcmprevAidvgkefgva | Capital: fragment |
| | with mutation | tntffkppcvsvyrcggccnseglqcmntstsylsktlfeitvplsqg | of propeptide |
| | (103-227)- | pkpvtisfanhtscrcmskldvyrqvhsiirrgshhhhhh | Capital, bold A: C |
| | VEGFC_C137A_His | | to A mutation |
| | | | Double underlined: |
| | | | GS linker |
| | | | Bold: his tag |
| 9 | >8 minor mature | TEETIKFAAahynteilksidnewrktqcmprevAidvgkefgva | Capital: fragment |
| | with mutation | tntffkppcvsvyrcggccnseglqcmntstsylsktlfeitvplsqg | of propeptide |
| | (103-227)- | pkpvtisfanhtscrcmskldvyrqvhsiirr | Capital, bold A: C |
| | VEGFC_C137A | | to A mutation |
| 10 | >9 major mature | ahynteilksidnewrktqcmprevAidvgkefgvatntffkppcvsv | Capital, bold A: C |
| | with mutation | yrcggccnseglqcmntstsylsktlfeitvplsqgpkpvtisfanht | to A mutation |
| | (112-227)- | scrcmskldvyrqvhsiirrgshhhhhh | Double underlined: |
| | VEGFC_C137A_His | | GS linker |
| | | | Bold: his tag |
| 11 | >9 major mature | ahynteilksidnewrktqcmprevAidvgkefgvatntffkppcvsv | Capital, bold A: C |
| | with mutation | yrcggccnseglqcmntstsylsktlfeitvplsqgpkpvtisfanht | to A mutation |
| | (112-227)- | scrcmskldvyrqvhsiirr | |
| | VEGFC_C137A | | |
| 12 | Propeptide | FESGLDLSDAEPDAGEATAYASKDLEEQLRSVS | |
| | | SVDELMTVLYPEYWKMYKCQLRKGGWQHNRE | |
| | | QANLNSRTEETIKFAA | |
| 13 | Fragment of | TEETIKFAA | |
| | Propeptide | | |
| 14 | VEGF-C consensus | ahynteilksidnewrktqcmprevXidvgkefgvatntffkppcvsv | wherein X is C or |
| | | yrcggccnseglqcmntstsylsktlfeitvplsqgpkpvtisfanht | A |
| | | scrcmskldvyrqvhsiirr | |
| 15 | VEGF-C | ahynteilksidnewrktqcmprevcidvgkefgvatntffkppcvsvy | |
| | | rcggccnseglqcmntstsylsktlfeitvplsqgpkpvtisfanhts | |
| | | crcmskldvyrqvhsiirr | |
| 16 | VEGF-C with | ahynteilksidnewrktqcmprevAidvgkefgvatntffkppcvsv | Capital, bold A: C |
| | mutation | yrcggccnseglqcmntstsylsktlfeitvplsqgpkpvtisfanht | to A mutation |
| | | scrcmskldvyrqvhsiirr | |

Studies show that placing adult macaques for 5 or 10 min in the Trendelenburg position, in which the body lies supine on a reclining table with the head approximately 30° below the feet, increases transduction of cervical relative to lumbar spinal cord after intrathecal infusion of AAV9. This suggests that gravity affects vector distribution and that positioning the head below the feet may improve delivery to the brain. Castle et al., Sci Adv 4(11): eaau9859 (2018).

In some embodiments, the subject is maintained in a position with hind limbs elevated, e.g. Trendelenburg like position, for about 5-10 minutes, about 10-30 minutes, about 30 minutes to 1 hour, about 1 to 2 hours, about 2-3 hours, or about 3-4 hours after the administration of the pharmaceutical composition. In preferred embodiments, the subject is maintained in a position with hind limbs elevated, e.g. Trendelenburg like position about 1 to 2 hours after the administration of the pharmaceutical composition.

In one aspect, the present disclosure provides methods for improving delivery of a pharmaceutical composition to the central nervous system. In some embodiments, the pharmaceutical composition comprises viral vectors, antibody, antisense oligonucleotide, or nanoparticles.

In some embodiments, the vector is a viral vector. In some embodiments, the vector is a viral vector used to deliver transgene sequence(s) to neuronal cells or tissue. Examples of viruses used for vectors include but are not limited to retroviruses, adenoviruses, lentiviruses, adeno-associated viruses, and other hybrid viruses. In some embodiments, the viral vector is an adeno-associated viral (AAV) vector, chimeric AAV vector, adenoviral vector, retroviral vector, lentiviral vector, DNA viral vector, herpes simplex viral vector, baculoviral vector, or any mutant or derivative thereof.

Without being bound by theory, viral vectors disclosed herein may insert their genomes into the host cell that they infect, thus delivering its nucleic acid sequence to the host. The viral genome inserted may be episomal or may be integrated into the chromosomes of the host cell at a site that may be random or targeted. In an embodiment, the vector is a viral vector used to deliver transgene sequences to cells. Examples of viruses used for vectors include but are not limited to retroviruses, adenoviruses, lentiviruses, adeno-associated viruses, and other hybrid viruses. Warnock et al., (2011) Methods Mol. Biol., 737:1-25. Lentivirus is a genus of retroviruses that can integrate significant amounts of viral DNA into a host cell, making them an efficient method of gene delivery. On the other hand, adenoviruses introduce genetic material that is not integrate into the chromosome of the host cell, thus reducing the risk of disrupting the host cell. In some embodiments, the viral vector is an adeno-associated viral (AAV) vector, chimeric AAV vector, adenoviral vector, retroviral vector, lentiviral vector, DNA viral vector, herpes simplex viral vector, baculoviral vector, or any mutant or derivative thereof.

In some embodiments, the vector comprising the transgene is or is derived from an adeno-associated virus (AAV). In some embodiments, the vector is a recombinant adeno-associated viral vector (rAAV). The rAAV genomes may comprise one or more AAV ITRs flanking a transgene sequence encoding a polypeptide (including, but not limited to, a hPGRN polypeptide) or encoding siRNA, shRNA, antisense, and/or miRNA directed at mutated proteins or control sequences of their genes. The transgene sequences are operatively linked, and may be linked by sequence encoding one or more protease cleavage sites or sequences encoding one or more self-cleaving peptides, or combinations thereof. In embodiments, the vectors additionally comprise other trasncriptional control elements such as those disclosed herein, e.g., promoter, enhancer, PRE, and/or polyA sequences that are functional in target cells to drive expression of the transgene sequence. The transgene sequence may also include intron sequences to facilitate processing of an RNA transcript when expressed in mammalian cells.

In various embodiments, the AAV vector, e.g., the rAAV vector, is a self-complementary AAV vector (scAAV). As used herein, “self-complementary” means the coding region has been designed to form an intra-molecular double-stranded template, e.g., in one or more inverted terminal repeats (ITRs). Without being bound by theory, a rate-limiting step for AAV genome often involves the second-strand synthesis since the typical AAV genome is a single-stranded DNA template. Ferrari et al, (1996) J. Virology, 70(5): 3227-34; Fisher et al, (1996) J. Virology, 70(1): 520-32. However, for scAAV genomes, upon infection, the two complementary halves of scAAV may associate to form one double stranded DNA (dsDNA) unit that is ready for replication and transcription rather than waiting for cell mediated synthesis of the second strand. In some embodiments, the rAAV vector disclosed herein is a scAAV vector and provides for faster and/or increased expression.

In some embodiments, the rAAV vectors disclosed herein lack one or more (e.g., all) AAV rep and/or cap genes. An AAV vector may comprise (e.g., in its ITRs) nucleic acid sequences (e.g., DNA) from any suitable AAV serotype. Suitable AAV serotypes include, but are not limited to, AAV serotypes AAV-1, AAV-2, AAV-3, AAV-4, AAV-5, AAV-6, AAV-7, AAV-8, AAV-9, AAV-10, AAV-11, AAV-12, AAVrh8, AAVrh10, AAV.Anc80, AAV.Anc80L65, AAV-DJ, and AAV-DJ/8, AAVrh37, AAV-DJ, AAV-DJ/8, AAV-PHP.B, AAV-PHP.B2, AAV-PHP.B3, AAV-PHP.A, AAV-PHP.eB, and AAV-PHP.S. For instance, an AAV vector, e.g., an scAAV vector, may comprise nucleic acid sequences from an AAV2, e.g., ITR sequences from an AAV2. An AAV vector, e.g., an scAAV vector, may also comprise nucleic acids from more than one serotype. The nucleotide sequences of the genomes of the AAV serotypes are known in the art. For example, the complete genome of AAV1 is provided in GenBank Accession No. NC_002077; the complete genome of AAV2 is provided in GenBank Accession No. NC 001401 and Srivastava et al., Virol., 45: 555-564 {1983): the complete genome of AAV3 is provided in GenBank Accession No. NC_1829; the complete genome of AAV4 is provided in GenBank Accession No. NC_001829; the AAV5 genome is provided in GenBank Accession No. AF085716; the complete genome of AAV-6 is provided in GenBank Accession No. NC_00 1862; at least portions of AAV7 and AAV8 genomes are provided in GenBank Accession Nos. AX753246 and AX753249, respectively; the AAV9 genome is provided in Gao et al., J. Virol., 78: 6381-6388 (2004); the AAV10 genome is provided in Williams, (2006)Mol. Ther., 13(1): 67-76; and the AAV11 genome is provided in Mori et al., (2004) Virology, 330(2): 375-383.

In some embodiments, functional inverted terminal repeat (ITR) sequences may be used to support, e.g., the rescue, replication and packaging of the AAV virion. Thus, an AAV vector disclosed herein may include sequences that in cis provide for replication and packaging (e.g., functional ITRs) of the virus. The ITRs can be but need not be the wild-type nucleotide sequences, and may be altered, e.g., by the insertion, deletion or substitution of nucleotides, so long as the sequences provide for functional rescue, replication and packaging. The ITRs may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, and AAV-11. The nucleotide sequences of the genomes of the AAV serotypes are known in the art. For example, the complete genome of AAV-1 is provided in GenBank Accession No. NC_002077; the complete genome of AAV-2 is provided in GenBank Accession No. NC 001401 and Srivastava et al., Virol., 45: 555-564 {1983): the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., (2004) J Virol., 78: 6381-6388; the AAV-10 genome is provided in Williams, (2006) Mol. Ther., 13(1): 67-76; and the AAV-11 genome is provided in Mori et al., (2004) Virology, 330(2): 375-383. In one embodiment, the vector is an AAV-9 vector, with AAV-2 derived ITRs.

In some embodiments, the rAAV vector disclosed herein comprise one or more ITRs, e.g., two ITRs, with one upstream and the other downstream of a transgene and/or the other nucleic acid elements discussed above. In some embodiments, a nucleic acid disclosed herein, e.g., in an scAAV vector, comprises a first ITR that is disposed 5′ and a second ITR that is disposed 3′ to the promoter, transgene, post-transcriptional regulatory element, and/or polyA, e.g., wherein the ITRs are independently 1, 2, 3, 4, 5, 6, 7, 8, 9, 10, 11, 12, 13, 14, 15, 16, 17, 18, 19, 20, 50, 100, 150, 200, 250 nucleotides 5′ and/or 3′ of the other elements. An ITR sequence may be wild-type, or it may comprise one or more mutations, e.g., as long as it retains one or more function of a wild-type ITR. In some embodiments, wild-type ITR may be modified to comprise a deletion of a terminal resolution site. In some embodiments, an scAAV as disclosed herein may comprise two ITR sequences, where both are wild-type, variant, or modified AAV ITR sequences. In some embodiments, at least one ITR sequence is a wild-type, variant or modified AAV ITR sequence. In some embodiments, the two ITR sequences are both wild-type, variant or modified AAV ITR sequences. In some embodiments, the “left” or 5′-ITR is a modified AAV ITR sequence that allows for production of self-complementary genomes, and the “right” or 3′-ITR is a wild-type AAV ITR sequence. In some embodiments, the “right” or 3′-ITR is a modified AAV ITR sequence that allows for the production of self-complementary genomes, and the “left” or 5′-ITR is a wild-type AAV ITR sequence. In some embodiments, the ITR sequences are wild-type, variant, or modified AAV2 ITR sequences. In some embodiments, at least one ITR sequence is a wild-type, variant or modified AAV2 ITR sequence. In some embodiments, the two ITR sequences are both wild-type, variant or modified AAV2 ITR sequences. In some embodiments, the “left” or 5′-ITR is a modified AAV2 ITR sequence that allows for production of self-complementary genomes, and the “right” or 3′-ITR is a wild-type AAV2 ITR sequence. In some embodiments, the “right” or 3′-ITR is a modified AAV2 ITR sequence that allows for the production of self-complementary genomes, and the “left” or 5′-ITR is a wild-type AAV2 ITR sequence. Exemplary sequences that may be used for one or more of the ITRs are described herein. In some embodiments, the AAV vector comprises SEQ ID NO: 12 and SEQ ID NO: 23. In some embodiments, the AAV vector comprises SEQ ID NO: 85 and SEQ ID NO: 90. Embodiments of AAV ITRs provided in WO/2019/094253 (PCT/US2018/058744), which is incorporated herein by reference in its entirety, may also be used for any AAV ITR disclosed herein.

In some embodiments, the rAAV vector lacks one or more (e.g., all) AAV rep and/or cap genes. An AAV vector may comprise (e.g., in its ITRs) nucleic acid sequences (e.g., DNA) from any suitable AAV serotype. Suitable AAV serotypes include, but are not limited to, AAV serotypes AAV-1, AAV-2, AAV-3, AAV-4, AAV-5, AAV-6, AAV-7, AAV-8, AAV-9, AAV-10 and AAV-11. For instance, an AAV vector, e.g., an scAAV vector, may comprise nucleic acid sequences from an AAV-2, e.g., ITR sequences from an AAV-2. An AAV vector, e.g., an scAAV vector, may also comprise nucleic acids from more than one serotype. GenBank Accession No. NC 001401 and Srivastava et al., Virol., 45: 555-564 {1983); GenBank Accession No. NC_1829; GenBank Accession No. NC_001829; GenBank Accession No. AF085716; GenBank Accession No. NC_00 1862; GenBank Accession Nos. AX753246 and AX753249; Gao et al., J. Virol., 78: 6381-6388 (2004); Williams, (2006) Mol. Ther., 13(1): 67-76; and Mori et al., (2004) Virology, 330(2): 375-383.

In some embodiments, functional inverted terminal repeat (ITR) sequences in a viral vector comprising the transgene may be used to support, e.g., the rescue, replication and packaging of the AAV virion. Thus, an AAV vector disclosed herein may include sequences that in cis provide for replication and packaging (e.g., functional ITRs) of the virus. The ITRs need not be the wild-type nucleotide sequences, and may be altered, e.g., by the insertion, deletion or substitution of nucleotides, so long as the sequences provide for functional rescue, replication and packaging. The ITRs may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10 and AAV-11. GenBank Accession No. NC_002077; GenBank Accession No. NC 001401 and Srivastava et al., Virol., 45: 555-564 {1983); GenBank Accession No. NC_1829; GenBank Accession No. NC_001829; GenBank Accession No. AF085716; GenBank Accession No. NC_00 1862; GenBank Accession Nos. AX753246 and AX753249, respectively; Gao et al., (2004) J. Virol., 78: 6381-6388; Williams, (2006) Mol. Ther., 13(1): 67-76; and Mori et al., (2004) Virology, 330(2): 375-383. In one embodiment, the vector is an AAV-9 vector, with AAV-2 derived ITRs.

In some embodiments, the AAV viral vector comprise a capsid protein derived from AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV 12, AAV16.3, AAV24.1, AAV27.3, AAV42.12, AAV42-lb, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV-17/rh.48, AAV-18/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r 11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAV A3.3, AAV A3.4, AAV A3.5, AAV A3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC 12, AAV-2-pre-miRNA-1O1, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPEN AAV 10 and/or Japanese AAV 10 serotypes, and variants thereof.

In some embodiments, the AAV viral vector comprise a capsid protein derived from AAV9.

In some embodiments, the AAV viral vector comprises a polynucleotide encoding a survival motor neuron (SMN) protein.

In some embodiments, the AAV viral vector comprises a polynucleotide encoding a methyl-CpG-binding protein 2 (MECP2) protein.

In some embodiments, the AAV viral vector comprises a polynucleotide encoding a short hairpin RNA (shRNA) targeting superoxide dismutase 1 (SOD1).

In some embodiments, the AAV viral vector comprises two ITRs (e.g. a modified AAV2 ITR and an unmodified AAV2 ITR), a promoter (e.g. a chicken beta-actin (CB) promoter), an enhancer (e.g. a cytomegalovirus (CMV) immediate/early enhancer), an intro (e.g. a modified SV40 late 16s intron), a polyadenylation signal (e.g. a bovine growth hormone (BGH) polyadenylation signal).

In some embodiments, the pharmaceutical composition comprises between 1×10¹⁰ and 1×10¹⁵ viral vector genomes, such as 1×10¹², 5×10¹², 1×10¹³, 5×10¹³, 1×10¹⁴, 5×10¹⁴, 1×10¹⁵ viral vector genomes.

In some embodiments, the pharmaceutical composition comprises between 1×10¹⁰ to 1×10¹⁵ vector genome per milliliter (vg/ml), such as 1×10¹², 5×10¹², 1×10¹³, 5×10¹³, 1×10¹⁴, 5×10¹⁴, 1×10¹⁵ vector genome per milliliter (vg/ml).

In various embodiments, the nucleic acids and vectors discussed herein may be present in one or more virus particle, such as a recombinant virus particle. Recombinant viruses are viruses generated by recombinant means. Various different viral types may be used, e.g., retroviruses, adenovirus, lentivirus, AAV, murine leukemia viruses, etc. Without being bound by theory, vectors delivered from retroviruses such as the lentivirus may provide for long-term gene transfer since they allow long-term, stable integration of a transgene and its propagation in daughter cells and may also provide low immunogenicity. Other suitable retroviruses include gammaretroviruses. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, e.g., in Tobias Maetzig et al., “Gammaretroviral Vectors: Biology, Technology and Application” Viruses. 2011 June; 3(6): 677-713. In some embodiments, the virus is a recombinant adenovirus comprising a nucleic acid or vector disclosed herein. In some embodiments, the virus is a recombinant AAV comprising a nucleic acid or vector disclosed herein.

In some embodiments, the nucleic acids or vectors disclosed herein are for use in the manufacture of a recombinant virus. In some embodiments, the nucleic acids or vectors disclosed herein are for use in the manufacture of an rAAV. Thus, also disclosed herein, in various embodiments, are virus compositions (also referred to as virions), e.g., rAAV virus compositions comprising a viral vector or nucleic acid disclosed above. In some embodiments, the recombinant virus is an adeno-associated virus (AAV) or any mutant or derivative thereof. In some embodiments, the recombinant virus is a chimeric AAV or any mutant or derivative thereof. In some embodiments, the recombinant virus is an adenovirus or any mutant or derivative thereof. In some embodiments, the recombinant virus is a retrovirus or any mutant or derivative thereof. In some embodiments, the recombinant virus is a lentivirus or any mutant or derivative thereof. In some embodiments, the recombinant virus is a DNA virus or any mutant or derivative thereof. In some embodiments, the recombinant virus is a herpes simplex virus or any mutant or derivative thereof. In some embodiments, the recombinant virus is a baculovirus or any mutant or derivative thereof.

In some embodiments, an AAV disclosed herein may comprise one or more AAV capsid proteins. AAV capsid proteins may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, AAV-11, AAV-12, AAVrh8, AAVfh10, AAV-DJ, AAV-DJ/8, AAV-PHP.B, AAV-PHP.B2, AAV-PHP.B3, AAV-PHP.A, AAV-PHP.eB, and AAV-PHP.S. In some embodiments, one or more capsid protein in an AAV is from an AAV-9. Without being bound by theory, typically in AAV, three capsid proteins, VP1, VP2 and VP3 multimerize to form the capsid. The polypeptide sequences of capsid proteins are known in the art, and can also be derived from the genome of the AAV. These can be used as exemplary capsids in the AAV virus compositions disclosed herein. For example, the complete genome of AAV-1 is provided in GenBank Accession No. NC_002077; the complete genome of AAV-2 is provided in GenBank Accession No. NC 001401 and Srivastava et al., Virol., 45: 555-564 {1983): the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J Virol., 78: 6381-6388 (2004); the AAV-10 genome is provided in Williams, (2006) Mol. Ther., 13(1): 67-76; and the AAV-11 genome is provided in Mori et al., (2004) Virology, 330(2): 375-383. Capsid proteins AAV-PHP.B, AAV-PHP.B2, AAV-PHP.B3, AAV-PHP.A, AAV-PHP.eB, or AAV-PHP.S are provided in Deverman et al., (2016) Nat. Biotech., 34: 204-209 and Chan et al., (2017) Nat. Neurosci., 20: 1172-1179. In some embodiments, the recombinant virus is an AAV comprising one or more AAV1, AAV2, AAV3, AAV4, AAV5, AAV6, AAV7, AAV 8, AAV9, AAV10, and AAV 11, AAV 12, AAVrh8, AAVrh10, AAV-DJ, AAV-DJ/8, AAV-PHP.B, AAV-PHP.B2, AAV-PHP.B3, AAV-PHP.A, AAV-PHP.eB, or AAV-PHP.S capsid serotype, or a functional variant thereof. In some embodiments, the recombinant virus is an AAV comprising a combination of capsids from more than one AAV serotype.

In some embodiments, AAV compositions disclosed herein comprise one or more cis-acting sequences directing viral DNA replication (rep), encapsidation/packaging and host cell chromosome integration are contained within the ITRs. In some embodiments, one or more of these sequences may also be present in trans rather than cis, e.g., on a separate plasmid during the virus manufacturing process in a host cell. Typically, three AAV promoters (named p5, p19, and p40 for their relative map locations) drive the expression of the two AAV internal open reading frames encoding rep and cap genes in wild-type virus. In some embodiments, one or more of these promoters and/or open reading frames are present in cis in an AAV vector and/or AAV virion disclosed herein, or are present on separate plasmids during the AAV virus manufacturing process, e.g., in a host cell producing the virus. The two rep promoters (p5 and p19), coupled with the differential splicing of the single AAV intron (at nucleotides 2107 and 2227), may result in the production of four rep proteins (rep 78, rep 68, rep 52, and rep 40) from the rep gene. Rep proteins possess multiple enzymatic properties that are ultimately responsible for replicating the viral genome. The cap gene is typically expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, (1992) Curr. Topics Microbiol. Imm., 158: 97-129.

In some embodiments, AAV compositions disclosed herein comprises engineered capsids with enhanced tropism to the human CNS or PNS. A variety of methods can be used for engineering the capsid proteins, including but not limited to, mutational methods, DNA barcoding, directed evolution, random peptide insertions, and capsid shuffling and/or chimeras.

Rational engineering and mutational methods have been used to direct AAV to a target tissue. In rational design, structure-function relationships are used to determine regions in which changes to the capsid sequence may be made. As non-limiting examples, surface loop structures, receptor binding sites, and/or heparin binding sites may be mutated, or otherwise altered, for rational design of recombinant AAV capsids for enhanced targeting to a target tissue. In one example of rational design, AAV capsids were modified by mutation of surface exposed tyrosines to phenylalanine, in order to evade ubiquitination, reduce proteasomal degradation and allow for increased AAV particle and viral genome expression (Lochrie M A, et al, J Virol. 2006 January; 80(2):821-34; Santiago-Ortiz J L and Schaffer D V, J Control Release, 2016 Oct. 28; 240:287-301, the contents of each of which are incorporated by reference in their entirety). Rational design also encompasses the addition of targeting peptides to a parent AAV capsid sequence, wherein the targeting peptide may have an affinity for a receptor of interest within a target tissue.

In certain embodiments, rational engineering and/or mutational methods are used to identify AAV capsids and/or targeting peptides having enhanced transduction of a target tissue (e.g., CNS or PNS).

Capsid shuffling, and/or chimeras describe a method in which fragments of at least two parent AAV capsids are combined to generate a new recombinant capsid protein, the number of parent AAV capsids used may be 2-20, or more than 20.

In certain embodiments, capsid shuffling is used to identify AAV capsids and/or targeting peptides having enhanced transduction of a target tissue (e.g., CNS or PNS).

Directed evolution involves the generation of AAV capsid libraries (˜10⁴-10⁸) by any of a variety of mutagenesis techniques and selection of lead candidates based on response to selective pressure by properties of interest (e.g., tropism). Directed evolution of AAV capsids allows for positive selection from a pool of diverse mutants without necessitating extensive prior characterization of the mutant library. Directed evolution libraries may be generated by any molecular biology technique known in the art, and may include, DNA shuffling, random point mutagenesis, insertional mutagenesis (e.g., targeting peptides), random peptide insertions, or ancestral reconstructions. AAV capsid libraries may be subjected to more than one round of selection using directed evolution for further optimization. Directed evolution methods are most commonly used to identify AAV capsid proteins with enhanced transduction of a target tissue. Capsids with enhanced transduction of a target tissue have been identified for the targeting human airway epithelium, neural stem cells, human pluripotent stem cells, retinal cells, and other in vitro and in vivo cells.

In certain embodiments directed evolution methods are used to identify AAV capsids and/or targeting peptides having enhanced transduction of a target tissue (e.g., CNS or PNS).

One method described for high-throughput characterization of the phenotypes of a large number of AAV serotypes is known as AAV Barcode-Seq (Adachi K et al, Nature Communications 5:3075 (2014), the contents of which are herein incorporated by reference in their entirety). In this next-generation sequence (NGS) based method, AAV libraries are created comprising DNA barcode tags, which can be assessed by multi-plexed Illumina barcode sequencing. This method can be used to identify AAV variants with altered receptor binding, tropism, neutralization and or blood clearance as compared to wild-type or non-variant sequences. Amino acids of the AAV capsid that are important to these functions can also be identified in this manner.

As described in Adachi et al 2014, AAV capsid libraries were generated, wherein each mutant carried a wild-type AAV2 rep gene and an AAV cap gene derived from a series of variants or mutants, and a pair of left and right 12-nucleotide long DNA bar-codes downstream of an AAV2 polyadenylation signal (pA). In this manner, 7 different DNA barcode AAV capsid libraries were generated. Capsid libraries were then provided to mice. At a pre-set timepoint, samples were collected, DNA extracted and PCR-amplified using AAV-clone specific virus bar codes and sample-specific bar code attached PCR primers. All the virus barcode PCR amplicons were Illumina sequenced and converted to raw sequence read number data by a computational algorithm. The core of the Barcode-Seq approach is a 96-nucleotide cassette comprising the DNA bar-codes (left and right) described above, three PCR primer binding sites and two restriction enzyme sites. As an exemplar, an AAV rep-cap genome was used, but the system can be applied to any AAV viral genome, including one devoid of rep and cap genes. The advantage of the Barcode Seq method is the collection of a large data set and correlation to desirable phenotype with few replicates and in a short period of time.

The DNA Barcode Seq method can be similarly applied to RNA.

In certain embodiments, the Barcode Seq method is used to identify AAV capsids and/or targeting peptides having enhanced transduction of a target tissue (e.g., CNS or PNS).

In some embodiments, insertion of targeting peptides into a parent AAV capsid sequence can be used to enhance targeting to CNS or PNS tissues. Disclosed herein are targeting peptides and associated AAV particles comprising a capsid protein with one or more targeting peptide inserts, for enhanced or improved transduction of a target tissue (e.g., cells of the CNS or PNS).

In certain embodiments, the targeting peptide may direct an AAV particle to a cell or tissue of the CNS. The cell of the CNS may be, but is not limited to, neurons (e.g., excitatory, inhibitory, motor, sensory, autonomic, sympathetic, parasympathetic, Purkinje, Betz, etc), glial cells (e.g., microglia, astrocytes, oligodendrocytes) and/or supporting cells of the brain such as immune cells (e.g., T cells). The tissue of the CNS may be, but is not limited to, the cortex (e.g, frontal, parietal, occipital, temporal), thalamus, hypothalamus, striatum, putamen, caudate nucleus, hippocampus, entorhinal cortex, basal ganglia, or deep cerebellar nuclei.

Targeting peptides of the present disclosure may be identified and/or designed by any method known in the art. As a non-limiting example, the CREATE system as described in Deverman et al., (Nature Biotechnology 34(2):204-209 (2016)) and in International Patent Application Publication Nos. WO2015038958 and WO2017100671, the contents of each of which are herein incorporated by reference in their entirety, may be used as a means of identifying targeting peptides, in either mice or other research animals, such as, but not limited to, non-human primates.

Non-limiting example of engineered AAV with enhanced targeting to CNS or PNS tissues can be found in US20180021364, US20210207167, US20210214749, US20210230632 and US20210277418, which are incorporated herein by reference in their entirety.

### Treatment of Disease

In various embodiments, the term “treating” comprises the step of administering an effective dose, or effective multiple doses, of a composition comprising a nucleic acid, a vector, a recombinant virus, or a pharmaceutical composition as disclosed herein, to an animal (including a human being) in need thereof. If the dose is administered prior to development of a disorder/disease, the administration is prophylactic. If the dose is administered after the development of a disorder/disease, the administration is therapeutic. In embodiments, an effective dose is a dose that detectably alleviates (either eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. The term encompasses but does not require complete treatment (i.e., curing) and/or prevention. In some embodiments, an effective dose comprises 1×10¹⁰ to 1×10¹⁵ vector genome per milliliter (vg/ml) of a virus as disclosed herein. In some embodiments, an effective dose comprises 1×10⁶ to 1×10¹⁰ plaque forming units per milliliter (pfu/ml) of a virus as disclosed herein. In some embodiments, an effective dose comprises 1×10⁶ to 1×10⁹ transducing units per milliliter (TU/ml) of a virus as disclosed herein. Examples of disease states contemplated for treatment are set out herein.

In some embodiments, a method of treating comprises delivering to a subject in need thereof a therapeutically effective amount of a nucleic acid disclosed herein. In some embodiments, a method of treating comprises delivering to a subject in need thereof a therapeutically effective amount of a vector disclosed herein. In some embodiments, a method of treating comprises delivering to a subject in need thereof a therapeutically effective amount of a recombinant virus disclosed herein. In some embodiments, a method of treating comprises delivering to a subject in need thereof a therapeutically effective amount of a pharmaceutical composition disclosed herein. In some embodiments, a nucleic acid, vector, recombinant virus, or pharmaceutical compositions disclosed herein is used in the manufacture of a medicament, for treating a subject in need thereof.

In various embodiments, the nucleic acid, vector, recombinant virus, or pharmaceutical composition disclosed herein may be delivered to the subject in need thereof by an intravenous administration, direct brain administration (e.g., intrathecal, intracerebral, and/or intraventricular administration), intranasal administration, intra-aural administration, or intra-ocular route administration, or any combination thereof. In some embodiments, the nucleic acid, vector, recombinant virus, or pharmaceutical composition is delivered by intrathecal administration. In some embodiments, the nucleic acid, vector, recombinant virus, or pharmaceutical composition is delivered by an intracerebral or intraventricular route of administration. In some embodiments, the administered nucleic acid, vector, recombinant virus, or pharmaceutical composition is ultimately delivered to the brain, spinal cord, peripheral nervous system, and/or CNS, either directly or by transfer after administration to a separate tissue or fluid, e.g., blood.

In one aspect, the methods and materials is indicated for treatment of nervous system disease or neurodegenerative disease, such as Rett Syndrome, Alzheimer's disease, Parkinson's disease, Huntington's disease, or for treatment of nervous system injury including spinal cord and brain trauma' injury, stroke, and brain cancers. In one embodiment, use of the methods and materials is indicated for treatment of spinal muscular atrophy (SMA).

There are four types of SMA, which are conventionally classified by age of onset and highest motor function achieved. All forms of SMA are autosomal recessive inheritance and caused by mutations of the survival motor neuron 1 (SMN1) gene. Humans also carry a second nearly identical copy of the SMN gene called SMN2. Lefebvre et al. “Identification and characterization of a spinal muscular atrophy-determining gene.” Cell, 80(1): 1 55-65. Monani et al. “Spinal muscular atrophy: a deficiency in a ubiquitous protein; a motor-neuron specific disease” Neuron, 48(6): 885-896. Both the SMN I and SMN2 genes express SMN protein, however SMN2 contains a translationaliy silent mutation in exon 7, which results in inefficient inclusion of exon 7 in SMN2 transcripts. Thus, SMN2 produces both full-length SMN protein and a truncated version of SMN lacking exon 7, with the truncated version as the predominant form. As a result, the amount of functional full-length protein produced by SMN2 is much less (by 70-90%) than that produced by SMN Lorson et al. “A single nucleotide in the SMN gene regulates splicing and is responsible for spinal muscular atrophy.” PNAS, 96(11) 6307-63 1. Monani et al, “A single nucleotide difference that alters splicing patterns distinguishes the SMA gene SMN1 from the copy gene SMN2.” Hum Mol Genet 8(7): 177-83. Although SMN2 cannot completely compensate for the loss of the SMN1 gene, patients with milder forms of SMA generally have higher SMN2 copy numbers. Lefebvre et al., ‘ Correlation between severity and SMN protein level in spinal muscular atrophy” Nat Genet 6(3):265-269. Park et al, ‘Spinal muscular atrophy: new and emerging insights from model mice.” Curr Neurol Neurosci Rep 10(2): 108-117. A caveat is that SMN2 copy number is not the sole phenotypic modifier. In particular, the c 859G C variant in exon 7 of the SMN2 gene has been reported as a positive disease modifier. Patient with this paiticular mutation have less severe disease phenotypes. Prior et al., “A positive modified of spinal muscular atrophy in the SMN2 gene.” Am J Hum Genet 85(3):408-413.

Type I SMA (also called infantile onset or Werdnig-Hoffmann disease) is when SMA symptoms are present at birth or by the age of 6 months. In this type, babies typically have low muscle tone (hypotonia), a weak cry and breathing distress. They often have difficulty swallowing and sucking, and do not reach the developmental milestone of being able to sit up unassisted. They often show one or more of the SMA symptoms selected from hypotonia delay in motor skills, poor head control, round shoulder posture and hypermobility of joints. Typically, these babies have two copies of the SMN2 gene, one on each chromosome 5. Over half of all new SMA cases are SMA type.

Type 11 or intermediate SMA is when SMA has its onset between the ages of 7 and months and before the child can stand or walk independently. Children with type 2 SMA generally have at least three SMN2 genes. Late-onset SMA (also known as types III and IV SMA, mild SMA, adult-onset SMA and Kugelberg-Welander disease) results in variable levels of weakness. Type III SMA has its onset after 18 months, and children can stand and walk independently, although they may require aid. Type IV SMA has its onset in adulthood, and people are able to walk during their adult years. People with types III or IV SMA generally have between four and eight SMN2 genes, from which a fair amount of full-length SMN protein can be produced.

In one embodiment, the term “treatment” comprises the step of administering intravenously, or via the intrathecal route, an effective dose, or effective multiple doses, of a composition comprising a rAAV as disclosed herein to an animal (including a human being) in need thereof. If the dose is administered prior to development of a disorder/disease, the administration is prophylactic. If the dose is administered after the development of a disorder/disease, the administration is therapeutic. In embodiments, an effective dose is a dose that alleviates (either eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. Examples of disease states contemplated for treatment are set out herein.

In one embodiment, the compositions comprising rAAV of the disclosure are administered intravenously to a patient in need thereof having an SMA type. In another embodiment, the compositions comprising rAAV of the disclosure are administered intrathecaily to a patient in need thereof having SMA types II, III, or IV.

A method of treating type 1 SMA in a patient in need thereof, by administering the AAV9 viral vector via an intrathecal or intravenous route s disclosed herein. In some embodiments, the patient is 0-9 months of age. In some other embodiments, the patient is 0-6 months of age. In some embodiments where the viral vector is used for treating type SMA in a patient, the weight of the patient is determined. In some embodiments, the patient has a body weight of less than 8.5 kg. In some embodiments, the patient has a body weight of more than 2.6 kg. In some embodiments, the patient has a body weight of 2.6-8.5 kg.

In some embodiments, the patient has mutations, e.g., a null mutation, in one copy of the SMN1 gene (encompassing any mutation that renders the encoded SM 1 nonfunctional). In some embodiments, the patient has mutations, e.g., a null mutation, in two copies of the SMN1 gene. In some embodiments, the patient has mutations, e.g., a null mutation, in all copies of the SMN1 gene. In some embodiments, the patient has a deletion in one copy of the SM 1 gene. In some embodiments, the patient has a deletion in two copies of the SMN1 gene. In some embodiments, the patient has biallelic SMN1 mutations, that is, either a deletion or substitution of SMN1 in both alleles of the chromosome. In some embodiments, the patient has at least one functional copy of the SMN2 gene. In some embodiments, the patient has at least two functional copies of the SMN2 gene. In some embodiments, the patient has at least two functional copies of the SMN2 gene. In some embodiments, the patient has at least three functional copies of the SMN2 gene. In some embodiments, the patient has at least four functional copies of the SMN2 gene. In some embodiments, the patient has at least five functional copies of the SMN2 gene. In some embodiments, the patient does not have a c.859G>C substitution in exon 7 of at least one copy of the SMN2 gene. In some embodiments, the genetic sequence of the SMN1 or SMN2 gene may be determined by full genome sequencing. In other embodiments, the genetic sequence and copy number of the SMN1 or SMN2 gene may be determined by high-throughput sequencing. In some embodiments, the genetic sequence and copy number of the SMN1 or SMN2 gene may be determined by microarray analysis. In some embodiments, the genetic sequence and copy number of the SMN1 or SMN2 gene may be determined by Sanger sequencing. In some embodiments, the copy number of the SMN1 or SMN2 gene may be determined by fluorescence in-situ hybridization (FISH).

In some embodiments, the patient shows one or more SMA symptoms. SMA symptoms can include hypotonia, delay in motor skills, poor head control, round shoulder posture and hypermobility of joints. In some embodiments, poor head control is determined by placing the patient in a ring sit position with assistance given a the shoulders (front and back). Head control is assessed by the patient's ability to hold the head upright. In some embodiments, spontaneous movement is observed when the patient is in a supine position and motor skills is assessed by the patient's ability to lift their elbows, knees, hands and feet off the surface. In some embodiments, the patient's grip strength is measured by placing a finger in the patient's palm and lifting the patient until their shoulder comes off the surface. Hypotonia and grip strength is measured by how soon/long the patient maintains grasp. In some embodiments, head control is assessed by placing the patient's head in a maximum available rotation and measuring the patient's ability to turn head back towards midline. In some embodiments, shoulder posture may be assessed by sitting patient down with head and trunk support, and observing if patient flexes elbows or shoulder to reach for a stimulus that s placed at shoulder level at arms length. In some embodiments, shoulder posture may also be assessed by placing patient in a side-lying position, and observing if patient flexes elbows or shoulder to reach for a stimulus that is placed at shoulder level at arms length. In some embodiments, motor skills are assessed by observing if the patients ilex their hips or knees when their foot is stroked, tickled or pinched. In some embodiments, shoulder flexion, elbow flexion, hip adduction, neck flexion, head extension, neck extension, and/or spinal incurvation may be assessed by know clinical measures, e.g., CHOP INTEND. Other SMA symptoms may be evaluated according to known clinical measures, e.g., CHOP INTEND.

In some embodiments, patients are treated after they show symptoms of type I SMA (e.g., one or more symptoms), as determined using one of the tests described herein In some embodiments, patients are treated before they show symptoms of type I SMA. In some embodiments, patients are diagnosed w th type I SMA based on genetic testing, before they are symptomatic, Combination therapies are also contemplated herein. Combination as used herein includes either simultaneous treatment or sequential treatments. Combinations of methods can include the addition of certain standard medical treatments (e.g., riluzole in ALS), as are combinations with novel therapies. For example, other therapies for SMA include antisense oligonucleotides (ASOs) that alter bind to pre-mRNA and alter their splicing patterns. Singh et a!, “A multi-exon-skipping detection assay reveals surprising diversity of splice isoforms of spinal muscular atrophy genes.” P os One, 7(ll):e49595. In one embodiment, nusinersen (U.S. Pat. Nos. 8,361,977 and 8,980,853, incorporated herein b reference) may be used. Nusinersen s an approved ASO that target intron 6, exon 7 or intron 7 of SM 2 pre-mRNA, modulating the splicing of SMN2 to more efficiently produce full-length SMN protein. In some embodiments, the method of treatment comprising the AAV9 viral vector is administered in combination with a muscle enhancer. In some embodiments, the method of treatment comprising the AAV9 viral vector is administered in combination with a neuroprotector. In some embodiments, the method of treatment comprising the AAV9 viral vector s administered in combination with an antisense oligonucleotide-based drug targeting SMN. n some embodiments, the method of treatment comprising the AAV9 viral vector is administered in combination with nusinersen. n some embodiments, the method of treatment comprising the AAV9 viral vector is administered in combination with a myostatin-inhibiting drug. In some embodiments, the method of treatment comprising the AAV9 viral vector is administered in combination with stamulumab.

While delivery to an individual in need thereof after birth is contemplated, intrauteral delivery' to a fetus is also contemplated.

Methods of treating type I SMA patients using the pharmaceutical compositions comprising the viral vector are contemplated. In some embodiments, the viral vector is formulated at a concentration of about 1-8×10¹³ AAV9 viral vector genomes/mL (vg/mL). In some embodiments, the viral vector is formulated at a concentration of about 1.7-2.3×10¹³ vg/mL. In some embodiments, the viral vector is formulated at a concentration of about 1.9-2.1×10¹³ vg/mL. In some embodiments, the viral vector is formulated at a concentration of about 2.0×10¹³ v mL.

In some embodiments where the viral vector is used for treating type I SMA in a patient, the AAV viral vector (e.g. AAV SMN) is administered to the patient at a dose of about 1.0-2.5×10¹⁴ vg/kg. In some embodiments where the viral vector is used for treating type I SMA in a patient, the AAV viral vector s administered to the patient at a dose of about 1. 0 14 vg/kg. In some embodiments where the viral vector is use for treating type I SMA in a patient, the AAV viral vector is infused into the patient over about 45-70 inin. In some embodiments where the viral vector is used for treating type I SMA in a patient, the AAV viral vector is infused into the patient over about 60 min. In some embodiments where the viral vector is used for treating type I SMA n a patient, the AAV viral vector is infused into the patient using an infusion pump, a peristaltic pump or any other equipment known in the art. In some embodiments where the viral vector is used for treating type I SMA in a patient, the AAV viral vector is infused into the patient using a syringe pump.

In one embodiment, the methods and materials described herein may be used for the treatment of neurodevelopmental disorders such as Rett Syndrome. Rett Syndrome is a rare neurological disorder first recognized in infancy, resulting from mutations in the MECP2 gene on the X chromosome in 90-95% of cases. Ruthie et al., “Rett syndrome is caused by mutations in X-linked MECP2, encoding methyl-CpG- bindin protein 2.” Nature Genetics, 23: 185-188. Boys who have only one copy of the X chromosome typically die shortly after birth, while girls who have two copies of the X chromosome usually have one functional copy of the gene. They begin to develop symptoms between 6-18 months, with hallmark symptoms like hand wringing or squeezing, clapping, rubbing, washing, or hand to mouth movements. The disease is progressive with significant disability that can include autistic-like behaviors, breathing irregularities, feeding and swallowing difficulties, growth retardation and seizures. There are 200 known mutations of the MECP2 gene, and depending on the level of X inactivation and dosage compensation, the severity of disease varies widely from patient to patient. Mouse studies show that MECP2 mutation does not cause neurons to die, suggesting that it is not a neurodegenerati ve disorder. Guy et ah “Reversal of Neurological Defects in a Mouse Model of Rett Syndrome. “Science, 315(5815)” 1:143-1 147.

For embodiments relating to Rett Syndrome, the rAAV (e.g. rAAV9) genome may encode, for example, methyl cytosine binding protein 2 (MeCP2). An exemplary AAV, e.g., scAAV9, construct comprising a polynucleotide encoding MeCP2 is provided in U.S. Pat. No. 9,415,121, the contents of which are hereby incorporated in their entirety. In some embodiments, an AAV construct comprising a polynucleotide encoding MeCP2 may be prepared using the methods disclosed herein. In some embodiments, these AAV constructs may be used to treat Rett Syndrome. In some embodiments, the MeCP2 AA V exhibits less than 10%, e.g., less than 7%, 5%, 4%, 3%, 2%, or 1% empty capsids. In some embodiments, the MeCP2 AAV exhibits low amounts of residual host cell protein, host cell DNA, piasmid DNA, and/or endotoxin, e.g., levels discussed herein for the preparation and purification of AAV vectors.

In one embodiment, the methods and materials described herein may be used for the treatment of ALS. ALS is a neurodegenerative disease resulting in progressive loss of motor neurons in the brain and spinal cord, with symptoms including the loss of ability to speak, eat, move and eventually breathe. The disease typically results in death within 3-5 years of diagnosis. While the cause of 90-95% of ALS causes is unknown, a subset of ALS is caused by genetic mutations in the superoxide dismutase 1 (SOD 1) gene, where a mutation causes a toxic dominant gain-of-function. Mouse studies show that SOD knockout does not result in disease and hence therapies that knock down levels of mutant SOD1 are thought to alleviate disease symptoms.

In some embodiments, the AAV vector encodes an shRNA targeting SOD 1 for ALS. An exemplary AAV, e.g., scAAV9, construct encoding shRNA for SOD1 is provided in WO201 503 1392 and US2016272976, the contents of which are hereby incorporated in their entirety. In some embodiments, an AAV construct encoding shRNA for SOD may be prepared using the methods disclosed herein. In some embodiments, these AAV constructs may be used to treat AL S In some embodiments, the SOD1 AAV exhibits less than 10%, e.g., less than 7%, 5%, 4%, 3%, 2%, or 1% empty capsids. in some embodiments, the SOD1 AAV exhibits low amounts of residual host cell protein, host cell DNA, plasmid DNA, and/or endotoxin, e.g., levels discussed herein for the preparation and purification of AAV vectors.

In some embodiments, the methods and materials described herein may be used for the treatment of neurodegenerative and/or neurodevelopmental disorders and improve the clinical trials as shown in Table 2.

TABLE 2
Gene Therapy for Neurodegenerative Disorders

| Gene | | | | | Primary | |
|---|---|---|---|---|---|---|
| therapy | Delivery route | identifier | Phase | Patients | endpoint | Duration |
| AAV2-NGF | IP (basal | NCT00087789 | 1 | Mild-to-moderate AD | Safety and | 2004-2010 |
| | forebrain) | | | | tolerability | |
| AAV2-NGF | IP (basal | NCT00876863 | 2 | Mild-to-moderate AD | Cognition | 2008-2015 |
| | forebrain) | | | | | |
| AAVrh.10- | IC | NCT03634007 | 1 | MCI or mild-to-severe | Safety | |
| hAPOE2 | | | | AD, APOE4 homozygotes, | | |
| | | | | aged ≥50 years | | |
| AAV-hTERT | IV + IT | NCT04133454 | 1 | AD or early dementia, | Safety and | |
| | | | | aged ≥40 years | tolerability | |
| ASO-MAPT | IT | NCT03186989 | 1/2 | Mild AD, aged 50-74 | Safety and | |
| | | | | years | tolerability | |
| AAV2-GAD | IP (subthalamic | NCT00195143 | 1 | Idiopathic PD, >5 years | Safety | 2003-2005 |
| | nucleus) | | | ofdisease duration | | |
| AAV2-GAD | IP (subthalamic | NCT00643890 | 2 | Idiopathic PD, >5 years | Disease severity | 2008-2010 |
| | nucleus) | | | ofdisease duration | andprogression | |
| Lentivirus-TH/ | IP (striatum) | NCT00627588 | 1/2 | Bilateral idiopathic PD, | Safety | 2008-2012 |
| AADC/CH1 | | | | age48-65 years, >5 | | |
| | | | | years of disease | | |
| | | | | duration | | |
| Lentivirus-TH/ | IP (striatum) | NCT01856439 | 1/2 | From NCT00627588 | Long-term | 2011-2022 |
| AADC/CH1 | | | | | safety and | |
| | | | | | tolerability | |
| AAV2-AADC | IP (striatum) | NCT00229736 | 1 | Mid- to late-stage PD, | Safety and | 2004-2013 |
| | | | | aged ≤75 years | tolerability | |
| AAV2-AADC | IP (striatum) | NCT01973543 | 1 | Idiopathic PD, >5 years | Safety and | 2013-2020 |
| | | | | ofdisease duration | tolerability | |
| AAV2-AADC | IP (putamen) | NCT02418598 | 1/2 | Idiopathic PD, aged ≤75 | Safety | |
| | | | | years | | |
| AAV2-AADC | IP (striatum) | NCT03065192 | 1 | Idiopathic PD, >5 years | Safety and | |
| | | | | ofdisease duration | suicide risk | |
| AAV2-AADC | IP (striatum) | NCT03562494 | 2 | PD, aged 40-75 years, >4 | Change in ON | 2018-2022 |
| | | | | years of disease | time without | |
| | | | | duration | troublesome | |
| | | | | | dyskinesia | |
| AAV2-NTN | IP (putamen) | NCT00252850 | 1 | Bilateral idiopathic PD, | Safety and | 2005-2007 |
| | | | | aged35-75 years, >5 years | tolerability | |
| | | | | of disease duration | | |
| AAV2-NTN | IP (putamen) | NCT00400634 | 2 | Bilateral idiopathic PD, | Disease severity | 2006-2008 |
| | | | | aged35-75 years, >5 years | andprogression | |
| | | | | of disease duration | | |
| AAV2-NTN | IP (substantia | NCT00985517 | 1/2 | Idiopathic PD, age 35-75 | Disease severity | 2009-2017 |
| | nigra + putamen) | | | | andprogression | |
| AAV2-GDNF | IP (putamen) | NCT01621581 | 1 | Idiopathic PD, >5 years | Safety and | 2013-2022 |
| | | | | ofdisease duration | tolerability | |
| AAV2-GDNF | IP (putamen) | NCT04167540 | 1 | PD, aged 35-75 years | Safety and | |
| | | | | | tolerability | |
| AAV9-GCase | IC | NCT04127578 | 1/2 | Moderate-to-severe PD, | Safety and | |
| | | | | at leastone pathogenic | immunogenicity | |
| | | | | GBA1 mutation | | |
| ASO-HTT | IT | NCT02519036 | 1/2 | Early manifest HD, | Safety and | 2015-2017 |
| | | | | aged 25-65 years | tolerability | |
| ASO-HTT | IT | NCT03342053 | 2 | From NCT02519036 | Safety and | 2017-2019 |
| | | | | | tolerability | |
| ASO-HTT | IT | NCT04000594 | 1 | Manifest HD, aged 25-65 | Pharmacokinetics | 2019-2021 |
| | | | | years | and pharmacodynamics | |
| ASO-HTT | IT | NCT03761849 | 3 | Manifest HD, aged 25-65 | Disease | 2019-2022 |
| | | | | years | progression | |
| | | | | | andtotal | |
| | | | | | functional | |
| | | | | | capacity | |
| ASO-HTT | IT | NCT03842969 | 3 | Patients who | Long-term | 2019-2024 |
| | | | | participated | safety and | |
| | | | | in priorrG6042 | tolerability, | |
| | | | | (ASO-HTT) studies | suicide risk, | |
| | | | | | cognition | |
| AAV5-miHTT | IP (striatum) | NCT04120493 | 1/2 | Early manifest HD, ≥40 | Safety | 2019-2026 |
| | | | | CAGrepeats in HTT | | |
| ASO-mHTT | IT | NCT03225833 | 1/2 | Early manifest HD, | Safety and | 2017-2020 |
| | | | | carrying a targeted | tolerability | |
| | | | | SNP rs362307, aged | | |
| | | | | 25-65 years | | |
| ASO-mHTT | IT | NCT03225846 | 1/2 | Early manifest HD, | Safety and | 2017-2020 |
| | | | | carrying a targeted | tolerability | |
| | | | | SNP rs362331, aged | | |
| | | | | 25-65 years | | |
| AAV9-SMN | IV | NCT02122952 | 1 | Type 1 SMA, <6 or 9 | Safety | 2014-2019 |
| | | | | months ofage | | |
| AAV9-SMN | IV | NCT03306277 | 3 | Type 1 SMA, ≤6 months | Independent | 2017-2019 |
| | | | | of age | sitting, | |
| | | | | | survival | |
| AAV9-SMN | IT | NCT03381729 | 1 | SMA with 3 copies of | Safety and | 2017-2021 |
| | | | | SMN2 (onset at <12 | tolerability | |
| | | | | months of age), aged | | |
| | | | | 6-60 months | | |
| AAV9-SMN | IV | NCT03461289 | 3 | Type 1 SMA, <6 months | Sitting without | 2018-2021 |
| | | | | of age | support | |
| AAV9-SMN | IV | NCT03505099 | 3 | Presymptomatic SMA | Independent | 2018-2021 |
| | | | | with 2 or 3copies of | sitting, standing | |
| | | | | SMN2, ≤6 weeks of age | without | |
| | | | | | support | |
| AAV9-SMN | IV | NCT03837184 | 3 | Type 1 SMA, <6 months | Sitting without | 2019-2021 |
| | | | | of age | support | |
| ASO-SMN2 | IT | NCT01494701 | 1 | Aged 2-14 years | Safety, | 2011-2013 |
| | | | | | tolerability and | |
| | | | | | pharmacokinetics | |
| ASO-SMN2 | IT | NCT01703988 | 1/2 | Aged 2-15 years | Safety and | 2012-2015 |
| | | | | | tolerability | |
| ASO-SMN2 | IT | NCT01780246 | 1 | From NCT01494701 | Safety and | 2013-2014 |
| | | | | | tolerability | |
| ASO-SMN2 | IT | NCT01839656 | 2 | Age ≤210 days, disease | Motor | 2013-2017 |
| | | | | onset between 21 and | milestones | |
| | | | | 180 days of age | | |
| ASO-SMN2 | IT | NCT02052791 | 1 | From NCT01703988, | Safety and | 2014-2017 |
| | | | | NCT01780246 | tolerability | |
| ASO-SMN2 | IT | NCT02292537 | 3 | Disease onset at >6 | Motor function | 2014-2017 |
| | | | | months ofage, aged | | |
| | | | | 2-12 years | | |
| ASO-SMN2 | IT | NCT02386553 | 2 | SMA with 2 or 3 copies of | respiratory | 2015-2025 |
| | | | | SMN2, ≤6 weeks | intervention, | |
| | | | | of age | survival | |
| ASO-SMN2 | IT | NCT02594124 | 3 | From NCT02193074, | Long-term | 2015-2023 |
| | | | | NCT02292537, | safety and | |
| | | | | NCT02052791, | tolerability | |
| | | | | NCT01839656, | | |
| | | | | NCT02462759 | | |
| ASO-SMN2 | IT | NCT04050852 | 1 | Aged 5-21 years | Pulmonary | 2019-2022 |
| | | | | | function | |
| ASO-SMN2 | IT | NCT04089566 | 2/3 | Child, adult, older adult | Safety and | 2020-2023 |
| | | | | | tolerability | |
| | | | | | with high | |
| | | | | | doses, | |
| | | | | | neuromuscular | |
| | | | | | function | |
| ASO-SOD1 | IT | NCT01041222 | 1 | Familial ALS with SOD1 | Safety, | 2010-2012 |
| | | | | mutation, ≥18 years | tolerability and | |
| | | | | of age | pharmacokinetics | |
| ASO-SOD1 | IT | NCT02623699 | 3 | Familial ALS with SOD1 | Safety, | 2016-2021 |
| | | | | mutation, ≥18 years | tolerability, | |
| | | | | of age | pharmacokinetics | |
| | | | | | and progression | |
| | | | | | of disability | |
| ASO-SOD1 | IT | NCT03070119 | 3 | From NCT02623699 | Long-term | 2017-2023 |
| | | | | | safety and | |
| | | | | | tolerability | |
| ASO-SOD1 | IT | NCT03764488 | 1 | Aged 18-65 years | Drug | 2018-2021 |
| | | | | | distribution in | |
| | | | | | theCNS | |
| ASO-C9orf72 | IT | NCT03626012 | 1 | ALS with C9orf72 | Safety and | 2018-2021 |
| | | | | mutation, ≥18 years | tolerability | |
| | | | | of age | | |
| ASO-C9orf72 | IT | NCT04288856 | 1 | From NCT03626012 | Long-term | 2020-2023 |
| | | | | | safety and | |
| | | | | | tolerability | |

### AAV Toxicity

While AAV9 vectors have shown remarkable potential for delivery to the CNS after systemic delivery, resulting in clinical success in pediatric patients with spinal muscular atrophy1, systemic injection of high doses of AAV vectors can lead to induction of a T-cell response that can eliminate transduced cells2. In monkeys, there is one report in which high systemic doses of an AAV9-like vector resulted in toxicity and death of the animal which was attributed to systemic inflammation. Hinderer et al., Hum. Gene. Ther. 29(3):285-298 (2018). The reason high doses are required is due to the relatively low efficiency of AAV on a per-vector genome copy basis to provide adequate transgene expression in a substantial number of target cells. As disclosed herein, the low efficiency of AAV could be remedied by enhancing glymphatic influx. Thus, the method described in the present disclosure allows more efficient transduction at lower doses and will result in better therapeutic efficacy while lowering safety issues, such as immunotoxicity. In one aspect. the present disclosure provides methods of reducing systemic exposure of a pharmaceutical composition that targets CNS of a subject in need thereof in order to reduce liver and/or dorsal root ganglion (DRG) toxicity in the subject, the method comprising administering to the subject an agent that enhances glymphatic influx in combination with the pharmaceutical composition.

### Reduce Variable Brain Distribution

As described herein, immunohistorchemistry for GFP expression in animals administered AAV9 encoding GFP shows variable levels of expression in sections of brain. By enhancing glymphatic influx at the time of intrathecal dosing, greater levels of vector in the interstitial fluid will be achieved resulting in improved and more uniform transduction of targeted cell types.

In one aspect, the present disclosure provides methods of reducing variable brain distribution of viral vectors among a population of patients treated with a pharmaceutical composition comprising the viral vectors, the method comprising administering to the subject an agent that enhances glymphatic influx in combination with the pharmaceutical composition.

### Pharmaceutical Compositions

In various embodiments, pharmaceutical compositions are disclosed. In some embodiments, a pharmaceutical composition comprises one or more nucleic acids, vectors and/or viruses disclosed herein. In some embodiments, the pharmaceutical composition comprises a pharmaceutically acceptable carrier.

The nucleic acids, vectors, and/or recombinant virus according to the present disclosure (e.g., viral particles) can be formulated to prepare pharmaceutically useful compositions. Exemplary formulations include, for example, those disclosed in U.S. Pat. Nos. 9,051,542 and 6,703,237, which are incorporated by reference in their entirety. The compositions of the disclosure can be formulated for administration to a mammalian subject, e.g., a human. In some embodiments, delivery systems may be formulated for intramuscular, intradermal, mucosal, subcutaneous, intravenous, intrathecal, injectable depot type devices, or topical administration.

In some embodiments, when the delivery system is formulated as a solution or suspension, the delivery system is in an acceptable carrier, e.g., an aqueous carrier. A variety of aqueous carriers may be used, e.g., water, buffered water, 0.8% saline, 0.3% glycine, hyaluronic acid and the like. These compositions may be sterilized and/or sterile filtered. The resulting aqueous solutions may be packaged for use as is, or lyophilized. In some embodiments, the lyophilized preparation is combined with a sterile solution prior to administration.

In some embodiments, the compositions, e.g., pharmaceutical compositions, may contain pharmaceutically acceptable auxiliary substances to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. In some embodiments, the pharmaceutical composition comprises a preservative. In some other embodiments, the pharmaceutical composition does not comprise a preservative.

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference as applicable, unless otherwise indicated. The following Examples are presented in order to more fully illustrate the preferred embodiments of the disclosure. These examples should in no way be construed as limiting the scope of the disclosed subject matter, which is defined by the appended claims.

### EXAMPLES

### Example 1: AAV Biodistribution in Non-Human Primate

### Methods

### Samples Evaluated

Tissues were collected from cynomolgus macaques dosed with 3.0×10¹³ vg of scAAV9-CB-GFP by intrathecal (IT) route through lumbar puncture (LP) or intracranial magnum (ICM) administration and compared to vehicle control animals. For dosing animals were placed under anesthesia with ketamine/Dexmedetomidine and maintained in the Trendelenburg position for 10 minutes followed by Atipamezole (α-2 adrenergic antagonist) as a reversal agent. Tissues were collected at necropsy and fixed in formalin prior to routine processing to paraffin for histological evaluation and molecular localization studies.

### Immunohistochemistry

Immunohistochemistry staining for GFP including the deparaffinization and antigen retrieval steps, were performed on a Ventana Discovery XT autostainer using standard Ventana Discovery XT reagents (Ventana, Indianapolis, IN). Slides were deparaffinized then submitted to heat-induced antigen retrieval by covering them with Cell Conditioning 1 (CC1/pH8) solution according to the standard Ventana retrieval protocol. Slides were incubated with the primary antibody (rabbit monoclonal anti-GFP antibody clone EPR14104-89 at 0.372 ug/mL) or a non-immune isotype-matched control (Rabbit monoclonal IgG clone DA1E at 0.372 ug/ml) as indicated in Table 3 for one hour. Visualization was obtained by incubation with the appropriate Ventana Discovery OmniMap HRP reagent as indicated below followed by Ventana Discovery ChromoMap 3,3′-Diaminobenzidine (DAB). Counterstaining was performed using Ventana Hematoxylin and Ventana Bluing reagent for 4 minutes each. Slides were dehydrated, cleared and coverslipped with a synthetic mounting medium. Immunohistochemistry slides were reviewed and assigned negative, minimal, moderate or strong reactivity scores based on absence of staining, <1% of the area stained, 1-10% of the tissue stained, 11-50% of the tissue stained or greater than 50% of the tissue stained respectively.

TABLE 3
Immunohistochemistry antibodies

| Antibody supplier/ | | Dilution/ |
|---|---|---|
| code | Clone | Concentration |
| Abcam/ab183735 | EPR 14104-89 | 0.372 ug/ml |
| Cell signaling | DA1E | 0.372 ug/ml |
| Technology/3900s | | |

### Image Analysis of GFP Immunohistochemical Stains

Image analysis was performed on 20× images scanned on an Aperio AT2 scanner (Leica Biosystems) using the HALO platform by Indica Labs (v3.0.311.149). Tissue was manually annotated to remove non-specific background staining. Area Quantification algorithm v2.1.3 using pixel based deconvolution was optimized to positive GFP immunohistochemistry signal and run on annotated images. Results were based on positive signal normalized to total tissue area resulting in % positive signal/total area. Analysis and graphs were performed on GraphPad Prism version 8.1.2. Differences in percent pixel positive area between IT and ICM groups were compared using the multiple Mann-Whitney testes of Groups analysis.

### In Situ Hybridization for Vector Sequence

In situ hybridization to detect GFP antisense (AS) and sense (S) sequences encoded in the AAV vector as well as Macaca fascicularis (Mf)-PPIB (AS) (positive control and tissue quality control) and DapB (AS) (negative control) genes was performed on select blocks using reagents and equipment supplied by Advanced Cell Diagnostics (ACD) (Hayward, CA) and Ventana Medical Systems (Roche, Tucson AZ). The in situ hybridization RNAScope® probes where designed by ACD. A list of probes is presented in Table 4. Positive PPIB and negative DAPB control probe sets were included to ensure mRNA quality and specificity, respectively. The hybridization method followed protocols established by ACD and Ventana systems using Ventana mRNA Red chromogens. Briefly, 5 μm sections were baked at 60 degrees for 60 minutes and used for hybridization. The deparaffinization and rehydration protocol was performed using a Sakura Tissue-Tek DR5 stainer with the following steps: 3 times xylene for 5 minutes each; 2 times 100% alcohol for 2 minutes; air dried for 5 minutes. Off-line manual pretreatment in 1× retrieval buffer at 98 to 104 degrees C. for 15 minutes. Optimization was performed by first evaluating PPIB and DAPB hybridization signal and subsequently using the same conditions for all slides. Following pretreatment the slides were transferred to a Ventana Ultra autostainer to complete the ISH procedure including protease pretreatment; hybridization at 43 degrees C. for 2 hours followed by amplification; and detection with HRP and hematoxylin counter stain.

TABLE 4
In situ hybridization probes

| Probe | Name | Vendor | Cat # | Comment |
|---|---|---|---|---|
| DapB | Dihydrodipicolinate | ACD | 312039 | Negative control |
| | reductase | | | probe |
| | Macaca fascicularis | | | Positive control |
| | | | | house |
| Mfa-PPIB | peptidylprolyl | ACD | 424149 | keeping gene |
| | isomerase B | | | probe |
| GFP- | Green Fluorescent | ACD | 400289 | Vector antisense |
| antisense | Protein | | | probe |
| GFP-sense | Green Fluorescent | ACD | 409978 | Vector sense |
| | Protein | | | probe |

### Results

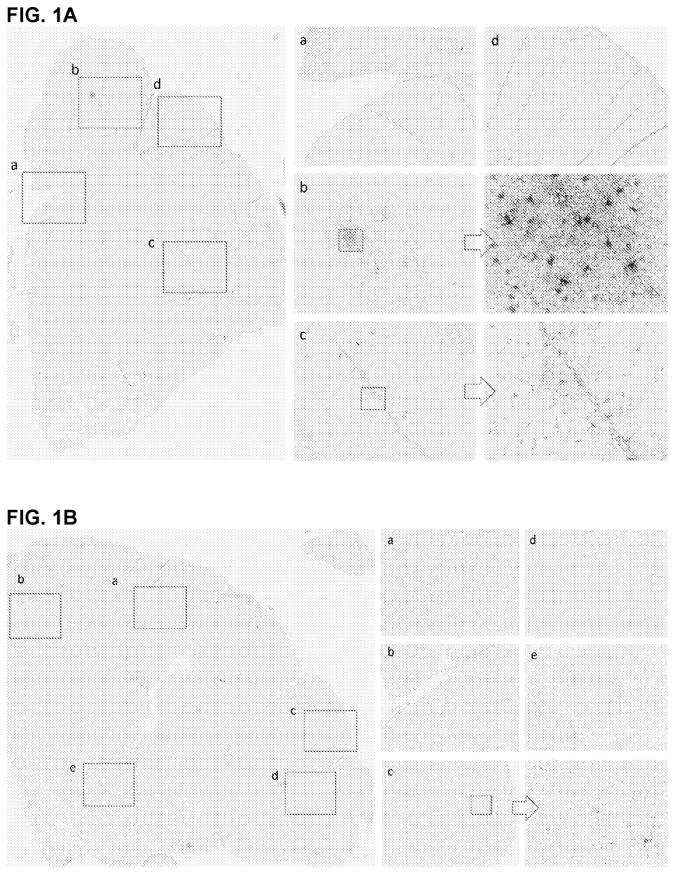

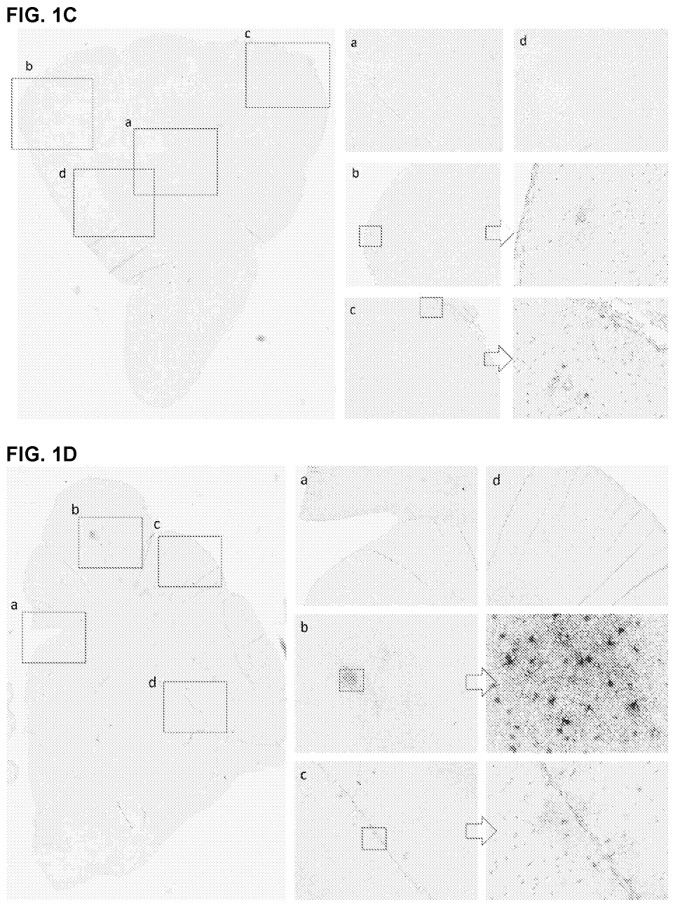

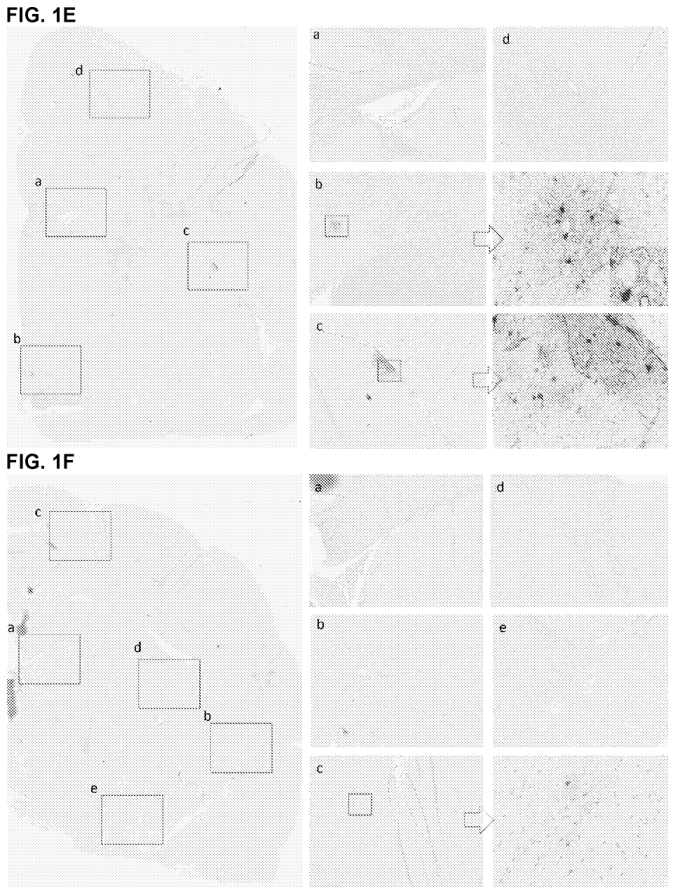

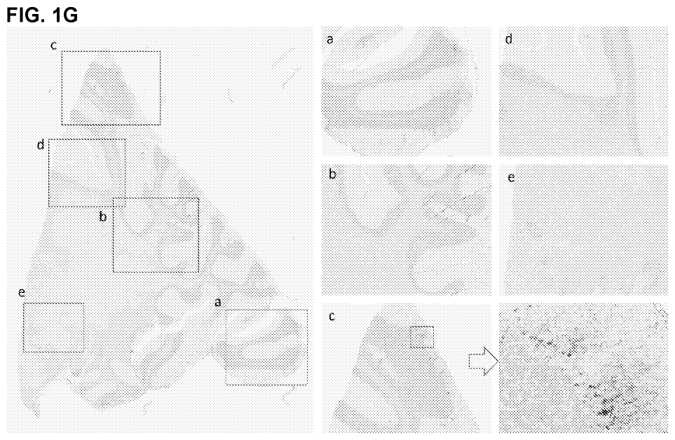

Immunohistochemistry for GFP protein expression was performed on select blocks of brain, spinal cord, lumbar dorsal root ganglion (DRG) and systemic tissues and scored for degree of GFP immunohistochemical staining (Table 5). Staining of control tissue produced no signal and no non-specific signal was observed with rabbit IgG control antibody DA1E. Compared to DRG and spinal cord overall lower and more variable levels of expression were detected in sections of brain from all animals evaluated administered scAAV9-CB-GFP (FIG. 1A through G). GFP protein detection was multifocal in distribution with significant regions of the brain parenchyma not demonstrating expression. In some regions strong expression was observed in the pia but absent in the underlying neuropil. Robust GFP expression was observed in systemic tissues such as liver and skeletal muscle. Despite administration of the vector directly into the CSF through the intrathecal route, these findings are consistent with a significant barrier to distribution of the vector to the interstitial fluid of the brain parenchyma a step required before the vector may interact with glycans and protein receptors on the surface of the targeted cell type.

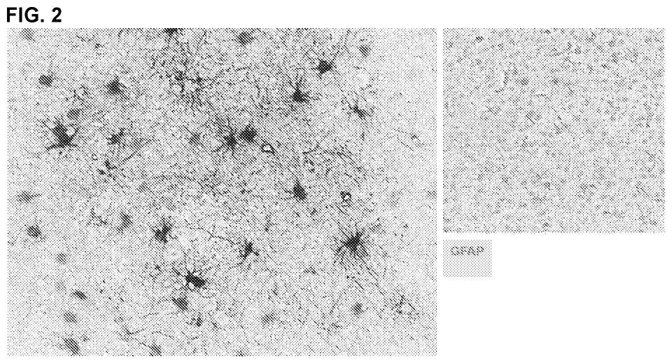

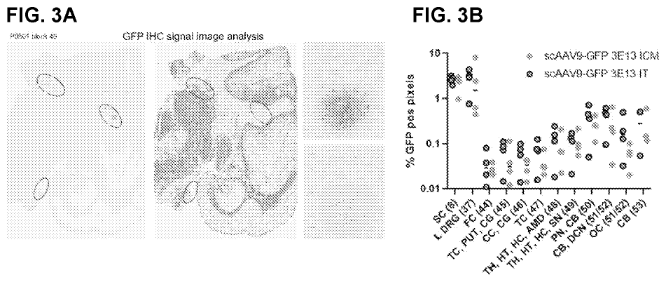

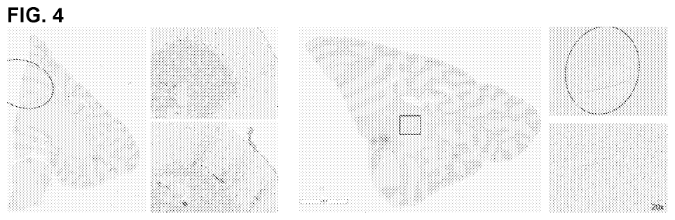

Morphologically the majority of cells expressing GFP protein appeared consistent with astrocytes. To verify this interpretation double label immunohistochemistry experiments were performed for the astrocyte marker GFAP and the GFP reporter (FIG. 2). These results corroborated the morphological interpretation and confirmed transduction and protein expression primarily of astrocytes. Quantitative image analysis was performed on sections of brain, spinal cord and DRG stained for GFP by immunohistochemistry and reported as percent GFP positive pixels (FIG. 3). The highest levels of expression were detected in the spinal cord and DRGs with lower levels of expression detected in brain. There were no statistically significant differences between LP IT and ICM animals dosed at 3.0×1013 vg/animal across the regions evaluated. Consistent with these findings limited GFP protein expression was observed in Purkinje neurons and neurons of the deep cerebellar nuclei (FIG. 4).

To confirm protein expression patterns, in situ hybridization was performed with GFP sense and antisense probes to detect vector sequence in select regions of brain. In situ hybridization detected similar patterns of vector localization compared to immunohistochemistry for GFP and often revealed signal in a vascular and perivascular pattern. Differences between LP IT and ICM dosed animals were not observed.

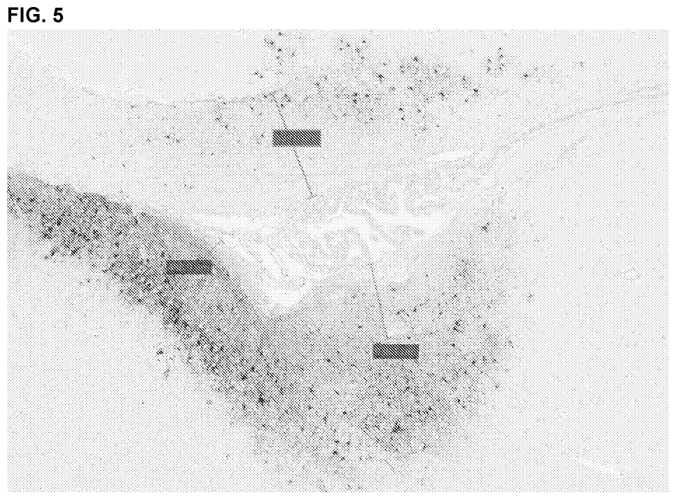

In addition to the multifocal perivascular distribution, limited periventricular GFP protein expression was evident in some animals. In this pattern, protein expression was detected primarily in astrocytes and was generally limited to the 500-1000 um of adjacent neuropil (FIG. 5). These findings are consistent with limited diffusion of the vector from ventricular CSF.

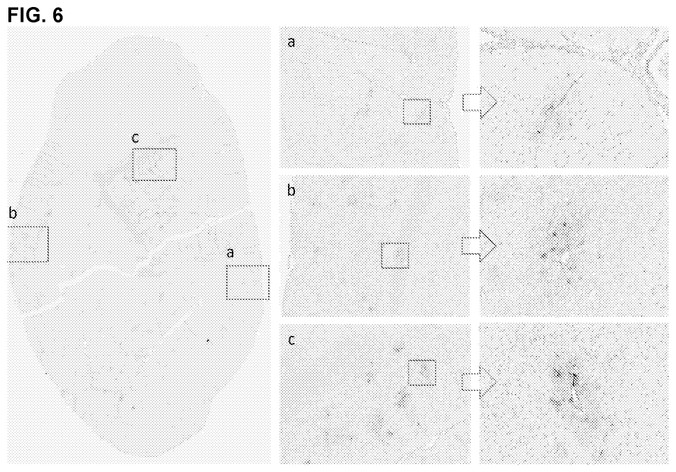

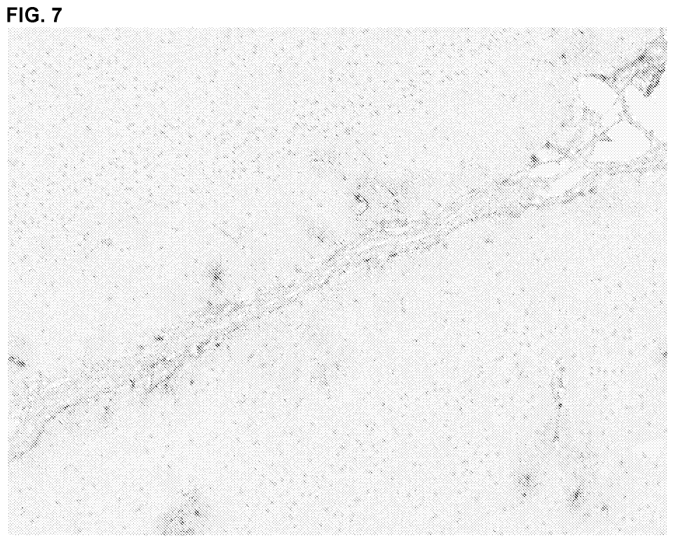

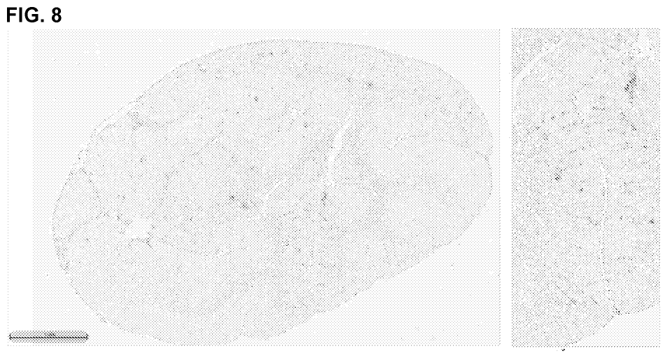

Further evaluation of the multifocal GFP expression pattern revealed that positive astrocytes often demonstrated a perivascular distribution along penetrating arterial vessels (FIGS. 6 and 7). Detection of GFP immunohistochemistry positive cells through image analysis highlighted the linear nature of distribution and expression along these vessels (FIG. 8). This perivascular transduction of astrocytes is consistent with the intrathecally administered vector reaching the brain parenchymal interstitial fluid through glymphatic influx.

TABLE 5
GFP immunohistochemistry scores on spinal, DRG and brain sections from control animal P0001
and animals with 3.0 × 10¹³ vg/animal of scAAV9-CB-GFP by the IT LP and ICM routes.

| Tissue | control | IT LP 3.0E13 | | | | ICM 3.0E13 | | | |
|---|---|---|---|---|---|---|---|---|---|
| (block number) | P0001 | P0301 | P0302 | P0303 | P0304 | P0501 | P0502 | P0503 | P0504 |
| L SC (8D) | neg | moderate | moderate | moderate | moderate | moderate | neg | mild | moderate |
| S DRG (37) | neg | moderate | Strong | moderate | mild | minimal | mild | mild | Strong |
| PFC (44) | neg | minimal | minimal | minimal | mild | minimal | minimal | minimal | minimal |
| TC, PUT, CG (45) | neg | mild | mild | neg | mild | mild | neg | mild | minimal |
| CC, CG (46) | neg | mild | mild | minimal | mild | minimal | minimal | mild | minimal |
| TC (47) | neg | mild | mild | minimal | mild | minimal | neg | mild | minimal |
| TH, HT, HC, AMD (48) | neg | mild | mild | minimal | mild | minimal | minimal | mild | minimal |
| TH, HT, HC, SN (49) | neg | mild | mild | minimal | mild | mild | n/p | mild | minimal |
| PN, CB (50) | neg | mild | mild | minimal | mild | mild | minimal | mild | moderate |
| CB, DCN (51 or 52) | neg | moderate | mild | minimal | moderate | mild | minimal | mild | minimal |
| OC (51 or 52) | neg | moderate | moderate | minimal | moderate | mild | minimal | mild | minimal |
| CB (53) | neg | mild | n/p | minimal | n/p | mild | n/p | mild | minimal |

LP IT, intrathecal; ICM, intracranial magna; neg, negative; L SC, lumbar spinal cord; S DRG, sacral dorsal root ganglion; PFC, prefrontal cortex; TC, temporal cortex; PUT, putamen; CG, cingulate gyrus; CC, corpus callosum; TH, thalamus; HT, hypothalamus; HC, hippocampus; AMD, amygdala; SN, substantia nigra; PN, pons; CB, cerebellum; DCN, deep cerebellar nuclei; OC: occipital cortex; n/p, not present; neg, negative.

The glymphatics are a recently recognized system by which CSF is drawn into the deeper regions of the brain along periarterial spaces formed by vessel adjacent astrocytes where CSF may exchange with the interstitial fluid prior to exiting the brain in an equivalent perivenule space. This system is thought to play a major role in the movement of fluid and removal macromolecules from the brain parenchyma. Larger particles such as lipoproteins which are of equivalent size to AAV vectors move through the glymphatic system. The GFP distribution patterns observed in this study are consistent with limited diffusion of vector across membranes lining the brain surface and vector entry occurring primarily through glymphatic influx.

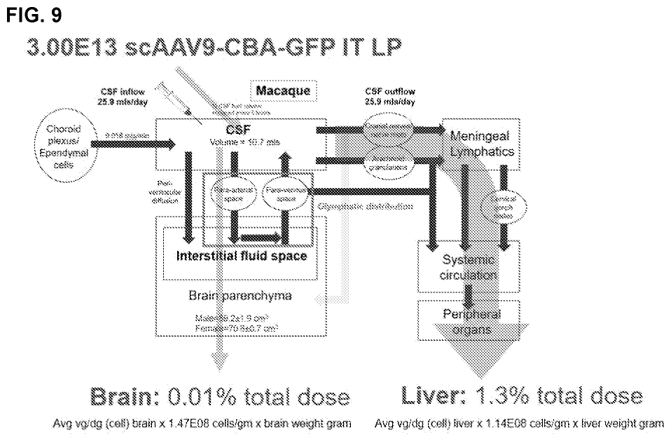

Based on these findings a model of AAV vector CNS and systemic distribution may be proposed following IT administration (FIG. 9). CSF is constantly being produced and in cynomolgus macaques has a half-life of approximately 5 hours before draining from the intrathecal space through arachnoid granulations and nerve roots where it enters meningeal lymphatics and subsequently the systemic circulation. Based on vector DNA copy number quantified in tissues only 0.01% of the total 3.0×10¹³ vector dose is detectable in brain at 1 month post dosing compared to 1.3% in liver following IT delivery. This is consistent with the majority of vector draining from the intrathecal space to the systemic circulation prior to exchange with interstitial fluid in the brain parenchyma through glymphatic influx. By enhancing glymphatic influx at the time of intrathecal dosing, greater levels of vector in the interstitial fluid will be achieved resulting in improved and more uniform transduction of targeted cell types. Moreover, reduction in vector distribution to systemic organs may reduce safety issues in these tissues.

### Example 2: Impact of Glymphatic Flow Modulation of AAV9 Brain Transduction after a Single Intrathecal Injection in Cynomologus Monkeys with a 4-Week Observation Period

Previous nonclinical NHP studies have demonstrated low and variable transduction of the brain parenchyma after intrathecal dosing of AAV gene therapy vectors. Using complementary molecular localization approaches (immunohistochemistry and in situ hybridization) to assess brain transduction, the vast majority of transduced cells detected in the parenchyma appear to be astrocytes located adjacent to the perivascular space, indicating that the vector may enter the brain parenchyma through the glymphatic system. A number of drivers may impact glymphatic influx such as time of administration relative to sleep cycle, anesthesia regimens, arterial pulse waves and peripheral osmolarity and influence the entry of vector particles within the interstitial space of the brain parenchyma. Manipulation of these factors may improve transduction levels of targeted cell types in the brain and reduce overall variability. Furthermore increasing distribution to and transduction of CNS tissues may also reduce systemic distribution and associated safety signals such as liver and dorsal root ganglion toxicity.

The objective of this study is therefore to explore dose timing, anesthetic regimes and plasma hyperosmolality to reduce variability in and increase levels of brain transduction following intrathecal injection of the AAV vector when administered as a single dose to cynomolgus monkeys. Monitoring of brain wave activity by EEG will be performed to assess anesthetic depth and improve the timing of dose administration relative to low frequency high amplitude delta wave patterns. After dosing, animals will be observed postdose for at least 4 weeks and alterations will be compared to a control group in which vector has been administered in a standard fashion.

TABLE 6
Group assignment and Dose Levels

| | Dose Level^{b} | Dose Concentration^{b} | Number of Animals |
|---|---|---|---|
| Group^{a} | (vg/animal) | (vg/mL) | Males |
| 1 | 3.0 × 10¹³ | TBD | 4 |
| 2 | 3.0 × 10¹³ | TBD | 4 |
| 3 | 3.0 × 10¹³ | TBD | 4 |

TBD = To be determined
Note:
Animals will be divided into 3 cohorts. Cohort 1 will consist of all animals in Group 1, Cohort 2 will consist of all animals in Group 2, and Cohort 3 will consist of all animals in Group 3
^{a}All groups will be dosed test article (scAAV9-CB-GFP).
^{b}Animals will be dosed at a volume of 2 mL/animal

### Dose Administration Rationale

The intrathecal injection route of administration was chosen because it is the intended human therapeutic route. It is the preferred route of administration for achieving broad transduction of the central nervous system while limiting systemic exposure.

### Dose Justification

A dose of 3e13 vg/animal has previously been used for characterizing the transduction profile of AAV9-CB-GFP within the brain parenchyma, and therefore it will be use as a benchmark. This dose level has generally been well-tolerated in past studies using a similar test article and no serious adverse event was reported. Previoustolerated findings at this dose included liver enzyme elevation and neuropathological changes in the dorsal root ganglia were observed (findings were identified as being related to the AAV platform).

### Species Selection

Cynomolgus monkeys historically have been used in AAV biodistribution and safety evaluation studies and are a non-clinical model of choice from a scientific point of view. The cynomolgus monkey was selected as the relevant species because of the similarity of CNS anatomy between monkeys and humans.

TABLE 7
Dose Administration

| Formulation | Dose formulations will be maintained at ambient conditions for at least |
|---|---|
| Handling | 30 minutes prior to dosing. |
| Frequency | Once on Day 1 of the dosing phase |
| | Day 1 of the dosing phase is defined as the day of dosing for each Cohort. |
| Dose Volume | 2 mL/animal |
| Dose Route | Intrathecal, slow bolus injection (using a 25 gauge × 1 inch Pencan Pencil |
| | Point Spinal Needles manufactured by B Braun and administered over 1 to |
| | 3 minutes, followed by a flush with 0.25 mL of 0.9% sterile saline) in the |
| | lumbar region |
| | Prior to injection, up to 1.0 mL of CSF will be removed and collected (See |
| | Cerebrospinal Fluid Collection section; the volume collected will be |
| | recorded). |
| | Cohort 1: Animals will be positioned in the Trendelenburg position for a |
| | period of 10 to 15 minutes after the IT injection. This timing may be adjusted |
| | based both on the ability to maintain acceptable vital signs of the animal. |
| | Cohorts 2 and 3: Animals will be positioned in the Trendelenburg position for |
| | a period of 1 to 2 hours after the IT injection. This timing may be adjusted |
| | based both on the ability to maintain acceptable vital signs of the animal. |
| | The location of each dose will be documented in the raw data. |
| Dose Site | Prior to dosing and as needed thereafter, the area used for dosing will be |
| Preparation | clipped free of hair. The injection site(s) will be marked and maintained |
| | as needed. The location of the dose will be documented in the raw data. |
| Dose Site | The dose site options are as follows. |
| Identification | Dose Site A: L5 to L6 |
| | If needed the following dose sites may be used, and will be documented. |
| | Dose Site B: L4 to L5 |
| | Dose Site C: L3 to L4 |
| | Dose Site D: L2 to L3 |
| Fasting Status | Animals will be fasted overnight prior to dosing. |

### Anesthesia Methods

### Method for Cohort 1

Prior to dosing animals will be anesthetized with ketamine (10 mg/kg) 10 to 15 prior to dosing followed by dexmedetomidine (0.02 mg/kg). After completion of dose administration, the animals will be maintained in dorsal recumbence with hind limbs elevated (Trendelenburg like position) for 10 to 15 minutes. Animals will be administered atipamezole (0.2 mg/kg IM). Dosing will occur at a standard time 8:00-10:00 AM.

### Method for Cohorts 2 and 3

Prior to dosing animals will be anesthetized with ketamine (10 mg/kg) 10 to 15 prior to dosing followed by dexmedetomidine (0.02 mg/kg) followed by sevoflurane inhalant anesthesia.

The depth of anesthesia will be monitored by EEG and dosing will happen when the deep anesthesia state (maximal delta power and minimal alpha power) will be reached.

For the cohort 3, intravenous injection of hypertonic saline (HTS) (NaCl 3% AT 2-3.5 ml/kg) will be performed and dosing will be done 5 minutes after HTS administration. Fluid and electrolytes require monitoring with the administration of all hypertonic fluids, with particular attention paid to serum sodium, potassium, and fluid ins/outs.

After completion of dose administration, the animals will be maintained in dorsal recumbence with hind limbs elevated (Trendelenburg like position) and kept anesthetized for a total procedure duration of 1 to 2 hours post dosing.

Dosing will occur at 2:00-4:00 PM.

### Clinical Observation

Animal health monitoring—At least twice daily (a.m. and p.m.); at least once on the day of transfer/termination.

Cageside observation—Once daily during the predose and dosing phases.

Postdose examinations—On the day of dosing for each dosed animal. Time point is 1 hour postdose. Observations will be based on the dosing completion time for each animal.

Body weight—Predose phase: At least once. Dosing phase: Once on Days 1, 8, 15, 22, and 28.

Food consumption—Daily during the predose and dosing phase except on day[s] of animal arrival/transfer or unless fasted for other study procedures if appropriate.

### Anti-AAV9 Capsid Immunogenicity Analysis

Serum sample will be collected at least once at predose phase, and prior to dosing on Day 1 and once on Days 8, 15, and 22 and on the day of scheduled euthanasia (only animals scheduled for sacrifice on that day) during dosing phase.

Serum samples will be analyzed for anti-AAV9 capsid immunogenicity when sufficient sample is available. In the event a planned test cannot be completed, the reason will be recorded.

### Biodistribution Analysis

Blood sample will be collected prior to dosing on Day 1 and once on Days 8, and on the day of scheduled euthanasia (only animals scheduled for sacrifice on that day) during dosing phase.

The blood cellular pellet and plasma will be analyzed for DNA (vector genome) by using a non-GLP method when samples are of sufficient volume. Instances of insufficient sample volume for analysis will be recorded.

### Nfl and GFAP Analysis

Plasma sample will be collected prior to dosing on Day 1 and once on Days 8, 15, 22 and 28 during dosing phase.

The plasma will be analyzed by using a non-GLP method for NfL and GFAP Analysis when samples are of sufficient volume. Instances of insufficient sample volume for analysis will be recorded.

Cerebrospinal fluid for Anti-AAV9 Capsid Immunogenicity, Biodistribution and NfL and GFAP Analysis

CSF sample will be collected prior to dosing on Day 1 and on the day of scheduled euthanasia (only animals scheduled for sacrifice on that day) during dosing phase.
Tube 1: CSF samples will be analyzed for DNA (vector genome) by using a non-GLP method when samples are of sufficient volume. Instances of insufficient sample volume for analysis will be recorded.
Tube 2: CSF samples will be analyzed by using a non-GLP method for NfL and GFAP Analysis when samples are of sufficient volume. Instances of insufficient sample volume for analysis will be recorded.
Tube 3 and 4: CSF samples will be analyzed for anti-AAV9 capsid immunogenicity (method information to be added by Amendment) when sufficient sample is available. In the event a planned test cannot be completed, the reason will be recorded.

It is understood that the examples and aspects described herein are for illustrative purposes only and that various modifications or changes in light thereof will be suggested to persons skilled in the art and are to be included within the spirit and purview of this application and scope of the appended claims.

In addition, where features or aspects of the disclosure are described in terms of Markush groups, those skilled in the art will recognize that the disclosure is also thereby described in terms of any individual member or subgroup of members of the Markush group.

All publications, patent applications, patents, and other references mentioned herein are expressly incorporated by reference in their entirety (or as context dictates), to the same extent as if each were incorporated by reference individually. In case of conflict, the present specification, including definitions, will control.

What is claimed is:

1. A method for improving delivery of a pharmaceutical composition to the central nervous system of a subject in need thereof, the method comprising administering to the subject an agent that enhances glymphatic influx in combination with the pharmaceutical composition.

2. The method of claim 1, wherein the agent is administered concurrently or sequentially with the pharmaceutical composition.

3. The method of claim 2, wherein the agent is administered prior to the administration of the pharmaceutical composition.

4. The method of claim 2, wherein the agent is administered after the administration of the pharmaceutical composition.

5. The method of any one of proceeding claims, wherein the pharmaceutical composition is administered by intrathecal (IT), intra-cisterna magna (ICM), and/or intracerebroventricular (ICV) administration.

6. The method of any one of proceeding claims, wherein the agent is administered by intravenous infusion, intravenous injection, inhalation, intraperitoneal, oral, subcutaneous or intramuscular routes.

7. The method of any one of the proceeding claims, wherein the agent promotes interstitial fluid circulation within the blood-brain barrier, e.g., wherein the agent comprises an Aquaporin 4 (AQP4) facilitator, e.g. TGN-073.

8. The method of any one of claims 1-6, wherein the agent comprises a compound that upregulates AQP4 expression (e.g. sevoflurane) or alters subcellular localization of AQP4.

9. The method of any one of claims 1-6, wherein the agent comprises an α-2 adrenergic agonist, e.g. clonidine, cizanidine, or dexmedetomidine (e.g. Precedex or Dexdomitor).

10. The method of any one of claims 1-6, wherein the agent comprises one or more FDA approved anesthetics that enhance glymphatic influx.

11. The method of claim 10, wherein the anesthetic is ketamine, dexmedetomidine, orxylazine, or a combination thereof.

12. The method of claim 10, wherein the agent comprises a combination of ketamine and dexmedetomidine.

13. The method of claim 12, wherein the subject is administered first with ketamine, followed by administration of the pharmaceutical composition, and followed by administration of dexmedetomidine.

14. The method of claim 13, wherein ketamine is administered about 1, 2, 3, 4, 5, 10, 15, 20, 25, 30, 35, 40, 45, 50, 55, or 60 minutes prior to, and preferably about 10 to 15 minutes prior to the administration of the pharmaceutical composition.

15. The method of any one of claims 12-14, wherein ketamine is administered at about 100 mg/kg, about 90 mg/kg, about 80 mg/kg, about 70 mg/kg, about 60 mg/kg, about 50 mg/k, about 40 mg/kg, about 30 mg/kg, about 20 mg/kg, about 10 mg/kg, about 9 mg/kg, about 8 mg/kg, about 7 mg/kg, about 6 mg/kg, about 5 mg/kg, about 4 mg/kg, about 3 mg/kg, about 2 mg/kg, about 1 mg/kg, preferable at about 10 mg/kg.

16. The method of any one of claims 12-14, wherein dexmedetomidine is administered at about 1 mg/kg, about 0.9 mg/kg, about 0.8 mg/kg, about 0.7 mg/kg, about 0.6 mg/kg, about 0.5 mg/k, about 0.4 mg/kg, about 0.3 mg/kg, about 0.2 mg/kg, about 0.1 mg/kg, about 0.09 mg/kg, about 0.08 mg/kg, about 0.07 mg/kg, about 0.06 mg/kg, about 0.05 mg/kg, about 0.04 mg/kg, about 0.03 mg/kg, about 0.02 mg/kg, about 0.01 mg/kg, about 0.009 mg/kg, about 0.008 mg/kg, about 0.007 mg/kg, about 0.006 mg/kg about 0.005 mg/kg, and preferable at about 0.02 mg/kg.

17. The method of any one of claims 12-16, wherein the subject is additionally administered sevolurane following the administration of dexmedetomidine.

18. The method of claim 17, wherein sevolurane is administered as an inhalant.

19. The method of any one of claims 1-6, wherein the agent induces plasma hypertonicity.

20. The method of claim 19, wherein the agent comprises hypertonic saline (e.g., Sodium chloride with or without sodium acetate) or mannitol.

21. The method of claim 20, wherein the agent comprises hypertonic saline with or without sodium acetate.

22. The method of claim 21, wherein the hypertonic saline is 2% NaCl, 3% NaCl, 5% NaCl, 7% NaCl or 23% NaCl, and preferably 3% NaCl.

23. The method of claim 21, wherein the 3% NaCl is administered at about 2-3.5 ml/kg.

24. The method of any one of claims 19-23, wherein the agent is administered by intravenous or infusion injection about 1, 2, 3, 4, 5, 6, 7, 8, 9, 10, 15, 20, preferably about 5 minutes prior or after to administration of the pharmaceutical composition, and optionally wherein the administration can be repeated.

25. The method of any one of claims 1-6, wherein the agent enhances glymphatic influx by increasing slow wave sleep.

26. The method of claim 25, wherein the agent is selected from the group consisting of:
Tiagabine, Gaboxadol, Gabapentin, Pregabalin, GHB, Ritanserin, Eplivanserin, Mirtazapine, Olanzapine, and Trazodone, or a combination thereof.

27. The method of any one of claims 1-6, wherein the agent comprises VEGF-C.

28. The method of any one of the proceeding claims, wherein the subject is maintained in a position with hind limbs elevated, e.g. Trendelenburg like position, for about 1-2 hours after the administration of the pharmaceutical composition.

29. The method of any one of the proceeding claims, wherein the pharmaceutical composition comprises viral vectors, antibody, antisense oligonucleotide, or nanoparticles.

30. The method of claim 29, wherein the pharmaceutical composition comprises adeno-associated virus (AAV) viral vectors.

31. The method of claim 30, wherein the AAV viral vector comprise a capsid protein derived from AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV 12, AAV16.3, AAV24.1, AAV27.3, AAV42.12, AAV42-lb, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV-17/rh.48, AAV-18/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r 11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAV A3.3, AAV A3.4, AAV A3.5, AAV A3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC 12, AAV-2-pre-miRNA-1O1, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPEN AAV 10 and/or Japanese AAV 10 serotypes, and variants thereof.

32. The method of claim 31, wherein the AAV viral vector comprise a capsid protein derived from AAV9.

33. The method of any one of claims 30-32, wherein the AAV viral vector comprises a polynucleotide encoding a survival motor neuron (SMN) protein.

34. The method of claims 30-32, wherein the AAV viral vector comprises a polynucleotide encoding a methyl-CpG-binding protein 2 (MECP2) protein.

35. The method of claim 30-32, wherein the AAV viral vector comprises a polynucleotide encoding a short hairpin RNA (shRNA) targeting superoxide dismutase 1 (SOD1).

36. The method of any one of claims 30-35, wherein the AAV viral vector comprises two ITRs (e.g. a modified AAV2 ITR and an unmodified AAV2 ITR), a promoter (e.g. a chicken beta-actin (CB) promoter), an enhancer (e.g. a cytomegalovirus (CMV) immediate/early enhancer), an intro (e.g. a modified SV40 late 16s intron), a polyadenylation signal (e.g. a bovine growth hormone (BGH) polyadenylation signal).

37. The method of any one of claims 30-36, wherein the pharmaceutical composition comprises between 1×10¹⁰ and 1×10¹⁵ viral vector genomes, such as 1×10¹², 5×10¹², 1×10¹³, 5×10¹³, 1×10¹⁴, 5×10¹⁴, 1×10¹⁵ viral vector genomes.

38. The method of any one of claims 30-36, wherein the pharmaceutical composition comprises between 1×10¹⁰ to 1×10¹⁵ vector genome per milliliter (vg/ml), such as 1×10¹², 5×10¹², 1×10¹³, 5×10¹³, 1×10¹⁴, 5×10¹⁴, 1×10¹⁵ vector genome per milliliter (vg/ml).

39. A method of treating a neurological disease, comprising administering a pharmaceutical composition to a subject in need thereof, wherein the pharmaceutical composition comprises an AAV encoding a gene associated with the neurological disease, wherein the administration of the pharmaceutical composition coincide with CSF influx during sleep cycle.

40. The method of claim 39, wherein the pharmaceutical composition is administered when the subject goes to sleep, e.g. as indicated by electroencephalogram (EEG) monitoring.

41. The method of claim 39 or 40, wherein the subject is administered a sleep enhancing drug in combination with the pharmaceutical combination.

42. The method of claim 41, wherein the sleep enhancing drug is selected from the group consisting of: Tiagabine, Gaboxadol, Gabapentin, Pregabalin, GHB, Ritanserin, Eplivanserin, Mirtazapine, Olanzapine, and Trazodone, or a combination thereof.

43. A method for improving the transduction efficiency and/or distribution of a neurodegenerative therapeutic agent in brain comprising administering the neurodegenerative therapeutic agent, in a subject in need thereof, in combination with a second agent that enhances glymphatic influx, to thereby improve transduction efficiency of the neurodegenerative therapeutic agent in the subject.

44. The method of claim 43, wherein the neurodegenerative therapeutic agent is a viral vector, an antibody, an antisense oligonucleotide, or a nanoparticle that targets the CNS.

45. The method of claim 43 or 44, wherein the second agent is administered concurrently or sequentially with the neurodegenerative therapeutic agent.

46. The method of claim 45, wherein the second agent is administered prior to the neurodegenerative therapeutic agent.

47. The method of claim 45, wherein the second agent is administered after the neurodegenerative therapeutic agent.

48. The method of any one of claims 43-47, wherein the neurodegenerative therapeutic agent is administered by intrathecal (IT) by intra-cisterna magna (ICM) and/or ICV administration by bolus, slow bolus and/or infusion through implanted intrathecal or intraventricular catheter.

49. The method of any one of claims 43-48, wherein the second agent is administered by intravenous infusion, intravenous injection and/or inhalation.

50. The method of any one of claims 43-49, wherein the second agent comprises an AQP4 facilitator, e.g. TGN-073.

51. The method of any one of claims 43-49, wherein the second agent comprises a compound that upregulates AQP4, e.g. sevoflurane.

52. The method of any one of any one of claims 43-49, wherein the second agent comprises an α-2 adrenergic agonist, e.g. clonidine, cizanidine, or dexmedetomidine (e.g. Precedex or Dexdomitor).

53. The method of any one of claims 43-49, wherein the second agent comprises one or more FDA approved anesthetics that enhance glymphatic influx.

54. The method of claim 53, wherein the anesthetic is ketamine, dexmedetomidine, orxylazine, or a combination thereof.

55. The method of claim 53, wherein the second agent comprises a combination of ketamine and dexmedetomidine.

56. The method of claim 55, wherein ketamine is administered about 1, 2, 3, 4, 5, 10, 15, 20, 25, 30, 35, 40, 45, 50, 55, or 60 minutes prior to, and preferably about 10 to 15 minutes prior to the administration of the neurodegenerative therapeutic agent.

57. The method of any one of claims 54-56, wherein ketamine is administered at about about 100 mg/kg, about 90 mg/kg, about 80 mg/kg, about 70 mg/kg, about 60 mg/kg, about 50 mg/k, about 40 mg/kg, about 30 mg/kg, about 20 mg/kg, about 10 mg/kg, about 9 mg/kg, about 8 mg/kg, about 7 mg/kg, about 6 mg/kg, about 5 mg/kg, about 4 mg/kg, about 3 mg/kg, about 2 mg/kg, about 1 mg/kg, preferable at about 10 mg/kg.

58. The method of any one of claims 54-56, wherein dexmedetomidine is administered at about 1 mg/kg, about 0.9 mg/kg, about 0.8 mg/kg, about 0.7 mg/kg, about 0.6 mg/kg, about 0.5 mg/k, about 0.4 mg/kg, about 0.3 mg/kg, about 0.2 mg/kg, about 0.1 mg/kg, about 0.09 mg/kg, about 0.08 mg/kg, about 0.07 mg/kg, about 0.06 mg/kg, about 0.05 mg/kg, about 0.04 mg/kg, about 0.03 mg/kg, about 0.02 mg/kg, about 0.01 mg/kg, about 0.009 mg/kg, about 0.008 mg/kg, about 0.007 mg/kg, about 0.006 mg/kg about 0.005 mg/kg, and preferable at about 0.02 mg/kg.

59. The method of any one of claims 54-58, wherein the subject is additionally administered sevolurane following the administration of dexmedetomidine.

60. The method of claim 59, wherein sevolurane is administered as an inhalant.

61. The method of any one of claims 43-49, wherein the second agent induces plasma hypertonicity.

62. The method of claim 61, wherein the second agent comprises hypertonic saline (e.g., Sodium chloride with or without sodium acetate) or mannitol.

63. The method of claim 62, wherein the second agent comprises hypertonic saline with or without sodium acetate.

64. The method of claim 63, wherein the hypertonic saline is 2% NaCl, 3% NaCl, 5% NaCl, 7% NaCl or 23% NaCl, and preferably 3% NaCl.

65. The method of claim 64, wherein the 3% NaCl is administered at about 2-3.5 ml/kg.

66. The method of any one of claims 61-65, wherein the agent is administered by intravenous or infusion injection about 1, 2, 3, 4, 5, 6, 7, 8, 9, 10, 15, 20, preferably about 5 minutes prior or after to administration of the pharmaceutical composition, and optionally wherein the administration can be repeated.

67. The method of any one of claims 43-49, wherein the second agent enhances glymphatic influx by increasing slow wave sleep.

68. The method of claim 67, wherein the second agent is selected from the group consisting of: Tiagabine, Gaboxadol, Gabapentin, Pregabalin, GHB, Ritanserin, Eplivanserin, Mirtazapine, Olanzapine, and Trazodone, or a combination thereof.

69. The method of any one of claims 43-49, wherein the second agent comprises VEGF-C.

70. The method of any one of claims 43-69, wherein the subject is maintained in a position with hind limbs elevated, e.g. Trendelenburg like position, for about 1 to 2 hours after the administration of the pharmaceutical composition.

71. The method of claims 43-70, wherein the neurodegenerative therapeutic agent is an adeno-associated virus (AAV) viral vector.

72. The method of claim 71, wherein the AAV viral vector comprise a capsid protein derived from AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV 12, AAV16.3, AAV24.1, AAV27.3, AAV42.12, AAV42-lb, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV-17/rh.48, AAV-18/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r 11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAV A3.3, AAV A3.4, AAV A3.5, AAV A3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC 12, AAV-2-pre-miRNA-1O1, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPEN AAV 10 and/or Japanese AAV 10 serotypes, and variants thereof.

73. The method of claim 72, wherein the AAV viral vector comprise a capsid protein derived from AAV9.

74. The method of any one of claims 71-73, wherein the AAV viral vector comprises a polynucleotide encoding a survival motor neuron (SMN) protein.

75. The method of claim 71-73, wherein the AAV viral vector comprises a polynucleotide encoding a methyl-CpG-binding protein 2 (MECP2) protein.

76. The method of claim 71-73, wherein the AAV viral vector comprises a polynucleotide encoding a short hairpin RNA (shRNA) targeting superoxide dismutase 1 (SOD1).

77. The method of any one of claims 71-76, wherein the AAV viral vector comprises two ITRs (e.g. a modified AAV2 ITR and an unmodified AAV2 ITR), a promoter (e.g. a chicken beta-actin (CB) promoter), an enhancer (e.g. a cytomegalovirus (CMV) immediate/early enhancer), an intro (e.g. a modified SV40 late 16s intron), a polyadenylation signal (e.g. a bovine growth hormone (BGH) polyadenylation signal).

78. The method of any one of claims 71-77, wherein the pharmaceutical composition comprises between 1×10¹⁰ and 1×10¹⁵ viral vector genomes, such as 1×10¹², 5×10¹², 1×10¹³, 5×10¹³, 1×10¹⁴, 5×10¹⁴, 1×10¹⁵ viral vector genomes.

79. The method of any one of claims 71-77, wherein the composition comprises between 1×10¹⁰ to 1×10¹⁵ vector genome per milliliter (vg/ml), such as 1×10¹², 5×10¹², 1×10¹³, 5×10¹³, 1×10¹⁴, 5×10¹⁴, 1×10¹⁵ vector genome per milliliter (vg/ml).

80. A method of increasing efficacy of an intrathecally delivered pharmaceutical composition, the method comprising administering to a subject in need thereof the pharmaceutical composition, in combination with an agent that enhances glymphatic influx.

81. The method of claim 80, wherein the agent is administered concurrently or sequentially with the composition.

82. The method of claim 81, wherein the agent is administered prior to the administration of the composition.

83. The method of claim 81, wherein the agent is administered after the administration of the composition.

84. The method of any one of claims 80-83, wherein the agent is administered by intravenous infusion, intravenous injection and/or inhalation.

85. The method of any one of claims 80-84, wherein the agent comprises an AQP4 facilitator, e.g. TGN-073.

86. The method of any one of claims 80-84, wherein the agent comprises a compound that upregulates AQP4, e.g. sevoflurane.

87. The method of any one of claims 80-84, wherein the agent comprises an α-2 adrenergic agonist, e.g. clonidine, cizanidine, or dexmedetomidine (e.g. Precedex or Dexdomitor).

88. The method of any one of claims 80-84, wherein the agent comprises one or more FDA approved anesthetics that enhance glymphatic influx.

89. The method of claim 88, wherein the anesthetic is ketamine, dexmedetomidine, orxylazine, or a combination thereof.

90. The method of claim 88, wherein the agent comprises a combination of ketamine and dexmedetomidine.

91. The method of claim 90, wherein the subject is administered first with ketamine, followed by administration of the pharmaceutical composition, and followed by administration of dexmedetomidine.

92. The method of claim 91, wherein ketamine is administered about 1, 2, 3, 4, 5, 10, 15, 20, 25, 30, 35, 40, 45, 50, 55, or 60 minutes prior to, and preferably about 10 to 15 minutes prior to the administration of the pharmaceutical composition.

93. The method of any one of claims 90-92, wherein ketamine is administered at 100 mg/kg, about 90 mg/kg, about 80 mg/kg, about 70 mg/kg, about 60 mg/kg, about 50 mg/k, about 40 mg/kg, about 30 mg/kg, about 20 mg/kg, about 10 mg/kg, about 9 mg/kg, about 8 mg/kg, about 7 mg/kg, about 6 mg/kg, about 5 mg/kg, about 4 mg/kg, about 3 mg/kg, about 2 mg/kg, about 1 mg/kg, preferable at about 10 mg/kg.

94. The method of any one of claims 90-92, wherein dexmedetomidine is administered at at about 1 mg/kg, about 0.9 mg/kg, about 0.8 mg/kg, about 0.7 mg/kg, about 0.6 mg/kg, about 0.5 mg/k, about 0.4 mg/kg, about 0.3 mg/kg, about 0.2 mg/kg, about 0.1 mg/kg, about 0.09 mg/kg, about 0.08 mg/kg, about 0.07 mg/kg, about 0.06 mg/kg, about 0.05 mg/kg, about 0.04 mg/kg, about 0.03 mg/kg, about 0.02 mg/kg, about 0.01 mg/kg, about 0.009 mg/kg, about 0.008 mg/kg, about 0.007 mg/kg, about 0.006 mg/kg about 0.005 mg/kg, and preferable at about 0.02 mg/kg.

95. The method of any one of claims 90-94, wherein the subject is additionally administered sevolurane following the administration of dexmedetomidine.

96. The method of claim 95, wherein sevolurane is administered as an inhalant.

97. The method of any one of claims 80-85, wherein the agent induces plasma hypertonicity.

98. The method of claim 97, wherein the agent comprises hypertonic saline or mannitol.

99. The method of claim 98, wherein the agent comprises hypertonic saline.

100. The method of claim 99, wherein the hypertonic saline is 3% NaCl.

101. The method of claim 100, wherein the 3% NaCl is administered at about 2-3.5 ml/kg.

102. The method of any one of claims 97-101, wherein the agent is administered by intravenous or infusion injection about 1, 2, 3, 4, 5, 6, 7, 8, 9, 10, 15, 20, preferably about 5 minutes prior or after to administration of the pharmaceutical composition, and optionally wherein the administration can be repeated.

103. The method of any one of claims 80-85, wherein the agent enhances glymphatic influx by increasing slow wave sleep.

104. The method of claim 103, wherein the agent is selected from the group consisting of: Tiagabine, Gaboxadol, Gabapentin, Pregabalin, GHB, Ritanserin, Eplivanserin, Mirtazapine, Olanzapine, and Trazodone, or a combination thereof.

105. The method of any one of claims 80-85, wherein the agent comprises VEGF-C.

106. The method of any one of claims 80-105, wherein the subject is maintained in a position with hind limbs elevated, e.g. Trendelenburg like position, for about 1-2 hours after the administration of the pharmaceutical composition.

107. The method of any one of claims 80-106, wherein the pharmaceutical composition comprises viral vectors, antibody, antisense oligonucleotide, or nanoparticles.

108. The method of claim 107, wherein the pharmaceutical composition comprises adeno-associated virus (AAV) viral vectors.

109. The method of claim 108, wherein the AAV viral vector comprise a capsid protein derived from AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV 12, AAV16.3, AAV24.1, AAV27.3, AAV42.12, AAV42-lb, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV-17/rh.48, AAV-18/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r 11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAV A3.3, AAV A3.4, AAV A3.5, AAV A3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC 12, AAV-2-pre-miRNA-1O1, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPEN AAV 10 and/or Japanese AAV 10 serotypes, and variants thereof.

110. The method of claim 109, wherein the AAV viral vector comprise a capsid protein derived from AAV9.

111. The method of any one of claims 108-110, wherein the AAV viral vector comprises a polynucleotide encoding a survival motor neuron (SMN) protein.

112. The method of claim 108-110, wherein the AAV viral vector comprises a polynucleotide encoding a methyl-CpG-binding protein 2 (MECP2) protein.

113. The method of claim 108-110, wherein the AAV viral vector comprises a polynucleotide encoding a short hairpin RNA (shRNA) targeting superoxide dismutase 1 (SOD1).

114. The method of any one of claims 108-113, wherein the AAV viral vector comprises two ITRs (e.g. a modified AAV2 ITR and an unmodified AAV2 ITR), a promoter (e.g. a chicken beta-actin (CB) promoter), an enhancer (e.g. a cytomegalovirus (CMV) immediate/early enhancer), an intro (e.g. a modified SV40 late 16s intron), a polyadenylation signal (e.g. a bovine growth hormone (BGH) polyadenylation signal).

115. The method of any one of claims 108-114, wherein the pharmaceutical composition comprises between 1×10¹⁰ and 1×10¹⁵ viral vector genomes, such as 1×10¹², 5×10¹², 1×10¹³, 5×10¹³, 1×10¹⁴, 5×10¹⁴, 1×10¹⁵ viral vector genomes.

116. The method of any one of claims 108-114, wherein the composition comprises between 1×10¹⁰ to 1×10¹⁵ vector genome per milliliter (vg/ml), such as 1×10¹², 5×10¹², 1×10¹³, 5×10¹³, 1×10¹⁴, 5×10¹⁴, 1×10¹⁵ vector genome per milliliter (vg/ml).

117. A method of reducing variable brain distribution of viral vectors among a population of patients treated with a pharmaceutical composition comprising the viral vectors, the method comprising administering to the subject an agent that enhances glymphatic influx in combination with the pharmaceutical composition.

118. The method of claim 117, wherein the agent is administered concurrently or sequentially with the composition.

119. The method of claim 118, wherein the agent is administered prior to the administration of the composition.

120. The method of claim 118, wherein the agent is administered after the administration of the composition.

121. The method of any one of claims 117-120, wherein the pharmaceutical composition is administered by intrathecal (IT) administration and/or by intra-cisterna magna (ICM).

122. The method of any one of claims 117-121, wherein the agent is administered by intravenous infusion, intravenous injection and/or inhalation.

123. The method of any one of claims 117-122, wherein the agent comprises an AQP4 facilitator, e.g. TGN-073.

124. The method of any one of claims 117-122, wherein the agent comprises a compound that upregulates AQP4, e.g. sevoflurane.

125. The method of any one of claims 117-122, wherein the agent comprises an α-2 adrenergic agonist, e.g. clonidine, cizanidine, or dexmedetomidine (e.g. Precedex or Dexdomitor).

126. The method of any one of claims 117-122, wherein the agent comprises one or more FDA approved anesthetics that enhance glymphatic influx.

127. The method of claim 126, wherein the anesthetic is ketamine, dexmedetomidine, orxylazine, or a combination thereof.

128. The method of claim 126, wherein the agent comprises a combination of ketamine and dexmedetomidine.

129. The method of claim 128, wherein the subject is administered first with ketamine, followed by administration of the pharmaceutical composition, and followed by administration of dexmedetomidine.

130. The method of claim 129, wherein ketamine is administered about 1, 2, 3, 4, 5, 10, 15, 20, 25, 30, 35, 40, 45, 50, 55, or 60 minutes prior to, and preferably about 10 to 15 minutes prior to the administration of the pharmaceutical composition.

131. The method of any one of claims 128-130, wherein ketamine is administered about 100 mg/kg, about 90 mg/kg, about 80 mg/kg, about 70 mg/kg, about 60 mg/kg, about 50 mg/k, about 40 mg/kg, about 30 mg/kg, about 20 mg/kg, about 10 mg/kg, about 9 mg/kg, about 8 mg/kg, about 7 mg/kg, about 6 mg/kg, about 5 mg/kg, about 4 mg/kg, about 3 mg/kg, about 2 mg/kg, about 1 mg/kg, preferable at about 10 mg/kg.

132. The method of any one of claims 128-130, wherein dexmedetomidine is administered at about 1 mg/kg, about 0.9 mg/kg, about 0.8 mg/kg, about 0.7 mg/kg, about 0.6 mg/kg, about 0.5 mg/k, about 0.4 mg/kg, about 0.3 mg/kg, about 0.2 mg/kg, about 0.1 mg/kg, about 0.09 mg/kg, about 0.08 mg/kg, about 0.07 mg/kg, about 0.06 mg/kg, about 0.05 mg/kg, about 0.04 mg/kg, about 0.03 mg/kg, about 0.02 mg/kg, about 0.01 mg/kg, about 0.009 mg/kg, about 0.008 mg/kg, about 0.007 mg/kg, about 0.006 mg/kg about 0.005 mg/kg, and preferable at about 0.02 mg/kg.

133. The method of any one of claims 128-132, wherein the subject is additionally administered sevolurane following the administration of dexmedetomidine.

134. The method of claim 133, wherein sevolurane is administered as an inhalant.

135. The method of any one of claims 117-122, wherein the agent induces plasma hypertonicity.

136. The method of claim 135, wherein the agent comprises hypertonic saline or mannitol.

137. The method of claim 136, wherein the agent comprises hypertonic saline.

138. The method of claim 137, wherein the hypertonic saline is 2% NaCl, 3% NaCl, 5% NaCl, 7% NaCl or 23% NaCl, and preferably 3% NaCl.

139. The method of claim 138, wherein the 3% NaCl is administered at about 2-3.5 ml/kg.

140. The method of any one of claims 135-139, wherein the agent is administered by intravenous or infusion injection about 1, 2, 3, 4, 5, 6, 7, 8, 9, 10, 15, 20, preferably about 5 minutes prior or after to administration of the pharmaceutical composition, and optionally wherein the administration can be repeated.

141. The method of any one of claims 117-122, wherein the agent enhances glymphatic influx by increasing slow wave sleep.

142. The method of claim 141, wherein the agent is selected from the group consisting of: Tiagabine, Gaboxadol, Gabapentin, Pregabalin, GHB, Ritanserin, Eplivanserin, Mirtazapine, Olanzapine, and Trazodone, or a combination thereof.

143. The method of any one of claims 117-122, wherein the agent comprises VEGF-C.

144. The method of any one of claims 117-143, wherein the subject is maintained in a position with hind limbs elevated, e.g. Trendelenburg like position, for about 1-2 hours after the administration of the pharmaceutical composition.

145. The method of any one of claims 117-144, wherein the pharmaceutical composition comprises adeno-associated virus (AAV) viral vectors.

146. The method of claim 145, wherein the AAV viral vector comprise a capsid protein derived from AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV 12, AAV16.3, AAV24.1, AAV27.3, AAV42.12, AAV42-lb, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV-17/rh.48, AAV-18/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r 11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAV A3.3, AAV A3.4, AAV A3.5, AAV A3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC 12, AAV-2-pre-miRNA-1O1, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPEN AAV 10 and/or Japanese AAV 10 serotypes, and variants thereof.

147. The method of claim 146, wherein the AAV viral vector comprise a capsid protein derived from AAV9.

148. The method of any one of claims 145-147, wherein the AAV viral vector comprises a polynucleotide encoding a survival motor neuron (SMN) protein.

149. The method of claim 145-147, wherein the AAV viral vector comprises a polynucleotide encoding a methyl-CpG-binding protein 2 (MECP2) protein.

150. The method of claim 145-147, wherein the AAV viral vector comprises a polynucleotide encoding a short hairpin RNA (shRNA) targeting superoxide dismutase 1 (SOD1).

151. The method of any one of claims 145-147, wherein the AAV viral vector comprises two ITRs (e.g. a modified AAV2 ITR and an unmodified AAV2 ITR), a promoter (e.g. a chicken beta-actin (CB) promoter), an enhancer (e.g. a cytomegalovirus (CMV) immediate/early enhancer), an intro (e.g. a modified SV40 late 16s intron), a polyadenylation signal (e.g. a bovine growth hormone (BGH) polyadenylation signal).

152. The method of any one of claims 145-151, wherein the pharmaceutical composition comprises between 1×10¹⁰ and 1×10¹⁵ viral vector genomes, such as 1×10¹², 5×10¹², 1×10¹³, 5×10¹³, 1×10¹⁴, 5×10¹⁴, 1×10¹⁵ viral vector genomes.

153. The method of any one of claims 145-151, wherein the composition comprises between 1×10¹⁰ to 1×10¹⁵ vector genome per milliliter (vg/ml), such as 1×10¹², 5×10¹², 1×10¹³, 5×10¹³, 1×10¹⁴, 5×10¹⁴, 1×10¹⁵ vector genome per milliliter (vg/ml).

154. A method of reducing systemic exposure of a pharmaceutical composition that targets CNS of a subject in need thereof in order to reduce liver and/or DRG toxicity in the subject, the method comprising administering to the subject an agent that enhances glymphatic influx in combination with the pharmaceutical composition.

155. The method of claim 154, wherein the agent is administered concurrently or sequentially with the composition.

156. The method of claim 155, wherein the agent is administered prior to the administration of the composition.

157. The method of claim 155, wherein the agent is administered after the administration of the composition.

158. The method of any one of claims 154-157, wherein the pharmaceutical composition is administered by intrathecal (IT) administration and/or by intra-cisterna magna (ICM).

159. The method of any one of claims 154-158, wherein the agent is administered by intravenous infusion, intravenous injection and/or inhalation.

160. The method of any one of claims 154-159, wherein the agent comprises an AQP4 facilitator, e.g. TGN-073.

161. The method of any one of claims 154-159, wherein the agent comprises a compound that upregulates AQP4, e.g. sevoflurane.

162. The method of any one of claims 154-159, wherein the agent comprises an α-2 adrenergic agonist, e.g. clonidine, cizanidine, or dexmedetomidine (e.g. Precedex or Dexdomitor).

163. The method of any one of claims 154-159, wherein the agent comprises one or more FDA approved anesthetics that enhance glymphatic influx.

164. The method of claim 163, wherein the anesthetic is ketamine, dexmedetomidine, orxylazine, or a combination thereof.

165. The method of claim 163, wherein the agent comprises a combination of ketamine and dexmedetomidine.

166. The method of claim 165, wherein the subject is administered first with ketamine, followed by administration of the pharmaceutical composition, and followed by administration of dexmedetomidine.

167. The method of claim 166, wherein ketamine is administered about 1, 2, 3, 4, 5, 10, 15, 20, 25, 30, 35, 40, 45, 50, 55, or 60 minutes prior to, and preferably about 10 to 15 minutes prior to the administration of the pharmaceutical composition.

168. The method of any one of claims 165-167, wherein ketamine is administered at about 100 mg/kg, about 90 mg/kg, about 80 mg/kg, about 70 mg/kg, about 60 mg/kg, about 50 mg/k, about 40 mg/kg, about 30 mg/kg, about 20 mg/kg, about 10 mg/kg, about 9 mg/kg, about 8 mg/kg, about 7 mg/kg, about 6 mg/kg, about 5 mg/kg, about 4 mg/kg, about 3 mg/kg, about 2 mg/kg, about 1 mg/kg, preferable at about 10 mg/kg.

169. The method of any one of claims 165-167, wherein dexmedetomidine is administered at about 1 mg/kg, about 0.9 mg/kg, about 0.8 mg/kg, about 0.7 mg/kg, about 0.6 mg/kg, about 0.5 mg/k, about 0.4 mg/kg, about 0.3 mg/kg, about 0.2 mg/kg, about 0.1 mg/kg, about 0.09 mg/kg, about 0.08 mg/kg, about 0.07 mg/kg, about 0.06 mg/kg, about 0.05 mg/kg, about 0.04 mg/kg, about 0.03 mg/kg, about 0.02 mg/kg, about 0.01 mg/kg, about 0.009 mg/kg, about 0.008 mg/kg, about 0.007 mg/kg, about 0.006 mg/kg about 0.005 mg/kg, and preferable at about 0.02 mg/kg.

170. The method of any one of claims 165-169, wherein the subject is additionally administered sevolurane following the administration of dexmedetomidine.

171. The method of claim 170, wherein sevolurane is administered as an inhalant.

172. The method of any one of claims 154-159, wherein the agent induces plasma hypertonicity.

173. The method of claim 172, wherein the agent comprises hypertonic saline (e.g., Sodium chloride with or without sodium acetate) or mannitol.

174. The method of claim 173, wherein the agent comprises hypertonic saline with or without sodium acetate.

175. The method of claim 174, wherein the hypertonic saline is 2% NaCl, 3% NaCl, 5% NaCl, 7% NaCl or 23% NaCl, and preferably 3% NaCl.

176. The method of claim 175, wherein the 3% NaCl is administered at about 2-3.5 ml/kg.

177. The method of any one of claims 172-176, wherein the agent is administered by intravenous or infusion injection about 1, 2, 3, 4, 5, 6, 7, 8, 9, 10, 15, 20, preferably about 5 minutes prior or after to administration of the pharmaceutical composition, and optionally wherein the administration can be repeated.

178. The method of any one of claims 154-159, wherein the agent enhances glymphatic influx by increasing slow wave sleep.

179. The method of claim 178, wherein the agent is selected from the group consisting of: Tiagabine, Gaboxadol, Gabapentin, Pregabalin, GHB, Ritanserin, Eplivanserin, Mirtazapine, Olanzapine, and Trazodone, or a combination thereof.

180. The method of any one of claims 154-159, wherein the agent comprises VEGF-C.

181. The method of any one of claims 154-180, wherein the subject is maintained in a position with hind limbs elevated, e.g. Trendelenburg like position, for about 1-2 hours after the administration of the pharmaceutical composition.

182. The method of any one of claims 154-181, wherein the pharmaceutical composition comprises viral vectors, antibody, antisense oligonucleotide, or nanoparticles.

183. The method of claim 182, wherein the pharmaceutical composition comprises adeno-associated virus (AAV) viral vectors.

184. The method of claim 183, wherein the AAV viral vector comprise a capsid protein derived from AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV 12, AAV16.3, AAV24.1, AAV27.3, AAV42.12, AAV42-lb, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV-17/rh.48, AAV-18/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r 11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAV A3.3, AAV A3.4, AAV A3.5, AAV A3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC 12, AAV-2-pre-miRNA-1O1, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPEN AAV 10 and/or Japanese AAV 10 serotypes, and variants thereof.

185. The method of claim 184, wherein the AAV viral vector comprise a capsid protein derived from AAV9.

186. The method of any one of claims 183-185, wherein the AAV viral vector comprises a polynucleotide encoding a survival motor neuron (SMN) protein.

187. The method of claim 183-185, wherein the AAV viral vector comprises a polynucleotide encoding a methyl-CpG-binding protein 2 (MECP2) protein.

188. The method of claim 183-185, wherein the AAV viral vector comprises a polynucleotide encoding a short hairpin RNA (shRNA) targeting superoxide dismutase 1 (SOD1).

189. The method of any one of claims 183-185, wherein vector comprises two ITRs (e.g. a modified AAV2 ITR and an unmodified AAV2 ITR), a promoter (e.g. a chicken beta-actin (CB) promoter), an enhancer (e.g. a cytomegalovirus (CMV) immediate/early enhancer), an intro (e.g. a modified SV40 late 16s intron), a polyadenylation signal (e.g. a bovine growth hormone (BGH) polyadenylation signal).

190. The method of any one of claims 183-189, wherein the pharmaceutical composition comprises between 1×10¹⁰ and 1×10¹⁵ viral vector genomes, such as 1×10¹², 5×10¹², 1×10¹³, 5×10¹³, 1×10¹⁴, 5×10¹⁴, 1×10¹⁵ viral vector genomes.

191. The method of any one of claims 183-189, wherein the composition comprises between 1×10¹⁰ to 1×10¹⁵ vector genome per milliliter (vg/ml), such as 1×10¹², 5×10¹², 1×10¹³, 5×10¹³, 1×10¹⁴, 5×10¹⁴, 1×10¹⁵ vector genome per milliliter (vg/ml).


SEQUENCE LISTING (16 sequences)

SEQ ID NO: 1 - protein, 419 aa, Homo sapiens
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 2 - protein, 204 aa, synthetic construct
FESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRGSHHHHHH

SEQ ID NO: 3 - protein, 196 aa, Homo sapiens
FESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRR

SEQ ID NO: 4 - protein, 124 aa, synthetic construct
AHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRGSHHHHHH

SEQ ID NO: 5 - protein, 116 aa, Homo sapiens
AHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRR

SEQ ID NO: 6 - protein, 133 aa, synthetic construct
TEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRGSHHHHHH

SEQ ID NO: 7 - protein, 125 aa, Homo sapiens
TEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRR

SEQ ID NO: 8 - protein, 133 aa, synthetic construct
TEETIKFAAAHYNTEILKSIDNEWRKTQCMPREVAIDVGKEFGVATNTFFKPPCVSVYRCGGCCNSEGLQCMNTSTSYLSKTLFEITVPLSQGPKPVTISFANHTSCRCMSKLDVYRQVHSIIRRGSHHHHHH

SEQ ID NO: 9 - protein, 125 aa, Homo sapiens
TEETIKFAAAHYNTEILKSIDNEWRKTQCMPREVAIDVGKEFGVATNTFFKPPCVSVYRCGGCCNSEGLQCMNTSTSYLSKTLFEITVPLSQGPKPVTISFANHTSCRCMSKLDVYRQVHSIIRR

SEQ ID NO: 10 - protein, 124 aa, synthetic construct
AHYNTEILKSIDNEWRKTQCMPREVAIDVGKEFGVATNTFFKPPCVSVYRCGGCCNSEGLQCMNTSTSYLSKTLFEITVPLSQGPKPVTISFANHTSCRCMSKLDVYRQVHSIIRRGSHHHHHH

SEQ ID NO: 11 - protein, 116 aa, Homo sapiens
AHYNTEILKSIDNEWRKTQCMPREVAIDVGKEFGVATNTFFKPPCVSVYRCGGCCNSEGLQCMNTSTSYLSKTLFEITVPLSQGPKPVTISFANHTSCRCMSKLDVYRQVHSIIRR

SEQ ID NO: 12 - protein, 80 aa, Homo sapiens
FESGLDLSDAEPDAGEATAYASKDLEEQLRSVSSVDELMTVLYPEYWKMYKCQLRKGGWQHNREQANLNSRTEETIKFAA

SEQ ID NO: 13 - protein, 9 aa, Homo sapiens
TEETIKFAA

SEQ ID NO: 14 - protein, 116 aa, Homo sapiens
AHYNTEILKSIDNEWRKTQCMPREVXIDVGKEFGVATNTFFKPPCVSVYRCGGCCNSEGLQCMNTSTSYLSKTLFEITVPLSQGPKPVTISFANHTSCRCMSKLDVYRQVHSIIRR

SEQ ID NO: 15 - protein, 116 aa, Homo sapiens; identical to SEQ ID NO: 5

SEQ ID NO: 16 - protein, 116 aa, Homo sapiens; identical to SEQ ID NO: 11